SEQUENCE LISTING (14 sequences)

SEQ ID NO: 1 - protein, 15 aa, Artificial sequence
  Synthetic
DLTKYPDANPNPNEQ

SEQ ID NO: 2 - protein, 15 aa, Artificial sequence
  Synthetic
SQEEGSSQGEDSVSG

SEQ ID NO: 3 - protein, 11 aa, Artificial sequence
  Synthetic
DSVSGSQRISS

SEQ ID NO: 4 - protein, 28 aa, Artificial sequence
  Synthetic
ISQEEGSSQGEDSDLTKYPDANPNPNEQ

SEQ ID NO: 5 - protein, 28 aa, Artificial sequence
  Synthetic
EDSVSGSQRISSIDLTKYPDANPNPNEQ

SEQ ID NO: 6 - protein, 1233 aa, Homo sapiens
MGFLKLIEIENFKSYKGRQIIGPFQRFTAIIGPNGSGKSNLMDAISFVLGEKTSNLRVKTLRDLIHGAPVGKPAANRAFVSMVYSEEGAEDRTFARVIVGGSSEYKINNKVVQLHEYSEELEKLGILIKARNFLVFQGAVESIAMKNPKERTALFEEISRSGELAQEYDKRKKEMVKAEEDTQFNYHRKKNIAAERKEAKQEKEEADRYQRLKDEVVRAQVQLQLFKLYHNEVEIEKLNKELASKNKEIEKDKKRMDKVEDELKEKKKELGKMMREQQQIEKEIKEKDSELNQKRPQYIKAKENTSHKIKKLEAAKKSLQNAQKHYKKRKGDMDELEKEMLSVEKARQEFEERMEEESQSQGRDLTLEENQVKKYHRLKEEASKRAATLAQELEKFNRDQKADQDRLDLEERKKVETEAKIKQKLREIEENQKRIEKLEEYITTSKQSLEEQKKLEGELTEEVEMAKRRIDEINKELNQVMEQLGDARIDRQESSRQQRKAEIMESIKRLYPGSVYGRLIDLCQPTQKKYQIAVTKVLGKNMDAIIVDSEKTGRDCIQYIKEQRGEPETFLPLDYLEVKPTDEKLRELKGAKLVIDVIRYEPPHIKKALQYACGNALVCDNVEDARRIAFGGHQRHKTVALDGTLFQKSGVISGGASDLKAKARRWDEKAVDKLKEKKERLTEELKEQMKAKRKEAELRQVQSQAHGLQMRLKYSQSDLEQTKTRHLALNLQEKSKLESELANFGPRINDIKRIIQSREREMKDLKEKMNQVEDEVFEEFCREIGVRNIREFEEEKVKRQNEIAKKRLEFENQKTRLGIQLDFEKNQLKEDQDKVHMWEQTVKKDENEIEKLKKEEQRHMKIIDETMAQLQDLKNQHLAKKSEVNDKNHEMEEIRKKLGGANKEMTHLQKEVTAIETKLEQKRSDRHNLLQACKMQDIKLPLSKGTMDDISQEEGSSQGEDSVSGSQRISSIYAREALIEIDYGDLCEDLKDAQAEEEIKQEMNTLQQKLNEQQSVLQRIAAPNMKAMEKLESVRDKFQETSDEFEAARKRAKKAKQAFEQIKKERFDRFNACFESVATNIDEIYKALSRNSSAQAFLGPENPEEPYLDGINYNCVAPGKRFRPMDNLSGGEKTVAALALLFAIHSYKPAPFFVLDEIDAALDNTNIGKVANYIKEQSTCNFQAIVISLKEEFYTKAESLIGVYPEQGDCVISKVLTFDLTKYPDANPNPNEQ

SEQ ID NO: 7 - protein, 12 aa, Artificial sequence
  Synthetic
SQEEGSSQGEES

SEQ ID NO: 8 - protein, 12 aa, Artificial sequence
  Synthetic
SQEEGGSQGEES

SEQ ID NO: 9 - protein, 11 aa, Artificial sequence
  Synthetic
DSVSGSQRTSS

SEQ ID NO: 10 - protein, 11 aa, Artificial sequence
  Synthetic
ESVSGSQRTSS

SEQ ID NO: 11 - protein, 16 aa, Artificial sequence
  Synthetic
CGSGSQEEGSSQGEDS

SEQ ID NO: 12 - protein, 15 aa, Artificial sequence
  Synthetic
CGSGDSVSGSQRISS

SEQ ID NO: 13 - protein, 4 aa, Artificial sequence
  Synthetic
CGSG

SEQ ID NO: 14 - protein, 12 aa, Artificial sequence
  Synthetic
SQEEGSSQGEDS

(19) United States
(12) Patent Application Publication
(10) Pub. No.: US 2013/0052668 A1
(43) Pub. Date: Feb. 28, 2013

(54) IDENTIFICATION AND USE OF BIOMARKERS FOR DETECTION AND QUANTIFICATION OF THE LEVEL OF RADIATION EXPOSURE IN A BIOLOGICAL SAMPLE

(71) Applicants: Amanda G. Paulovich, Seattle, WA (US); Richard G. Ivey, Federal Way, WA (US)

(72) Inventors: Amanda G. Paulovich, Seattle, WA (US); Richard G. Ivey, Federal Way, WA (US)

(21) Appl. No.: 13/695,262

(22) PCT Filed: Apr. 29, 2011
(86) PCT No.: PCT/US2011/034656

§ 371 Date: Oct. 29, 2012

Related U.S. Application Data
(60) Provisional application No. 61/330,273, filed on Apr. 30, 2010.

(51) Int. Cl. G01N 33/566; C12M 1/34; G01N 33/53

(52) U.S. Cl. 435/7.92; 436/501; 435/7.9; 422/69; 435/287.2

(57) ABSTRACT

The present invention provides methods, reagents, kits and devices for carrying out a diagnostic assay for use in assessing the exposure to ionizing radiation in a biological sample of interest.

### CROSS-REFERENCE TO RELATED APPLICATION

This application claims priority to U.S. Provisional Application Ser. No. 61/330,273, filed Apr. 30, 2010, the disclosure of which is incorporated herein by reference.

### STATEMENT OF GOVERNMENT LICENSE RIGHTS

This invention was made with Government support under NIH U19 AI067770 awarded by the National Institutes of Health. The Government has certain rights in the invention.

### STATEMENT REGARDING SEQUENCE LISTING

The sequence listing associated with this application is provided in text format in lieu of a paper copy and is hereby incorporated by reference into the specification. The name of the text file containing the sequence listing is: 36765_SEQ_FINAL.txt. The text file is 15 KB; was created on Apr. 29, 2011; and is being submitted via EFS-Web with the filing of the specification.

### FIELD OF THE INVENTION

This invention relates to methods, reagents, kits and devices for use in assessing the exposure to ionizing radiation in a biological sample.

### BACKGROUND

In the event of a nuclear or radiological incident in a heavily populated area, the surge demand for medical evaluation will likely overwhelm our emergency care system, compromising our ability to care for victims with life-threatening injuries or exposures. Historically during such events, much of the surge in demand has come from individuals who were neither exposed to radiation nor required acute medical intervention. Rather, most individuals presenting for care have been victims of mass panic. Media coverage of actual and potential nuclear attacks or accidents has left the public with a sensationalized fear of such events, focused on catastrophic outcomes. As a result, disaster planning for a radiological incident must anticipate widespread panic. Indeed, many experts label these types of attacks “weapons of mass disruption (Levi, M. A., and H. C. Kelly, “Weapons of Mass Disruption,” Sci Am 287:76-81 (2002)),” and it is the disruptive potential that makes radiological terrorism appealing to terrorists. The scenarios of concern range from the use of a radiological dispersion device (RDD), with a relatively limited number of casualties incurred, to the detonation of improvised nuclear devices (IND) and explosions or leaks at nuclear power plants, where large numbers of casualties might be anticipated.

Engel et al. (Engel, C. C., et al., “Terrorism, Trauma, and Mass Casualty Triage: How Might We Solve the Latest Mind Body Problem?” Biosecur Bioterror 5:155-163 (2007)) described “mass idiopathic illness,” in which during the immediate aftermath of a radiological or nuclear attack a large number of individuals present to triage points with acute anxiety and idiopathic physical symptoms. “In the event that this phenomenon occurs, it could result in surges in demand for medical evaluations that may disrupt triage systems and endanger lives (Engel, C. C., et al., “Terrorism, Trauma, and Mass Casualty Triage: How Might We Solve the Latest Mind Body Problem?” Biosecur Bioterror 5:155-163 (2007)).” Indeed, historically there are many examples of mass idiopathic illness (Bartholomew, R. E., and S. Wessely, “Protean Nature of Mass Sociogenic Illness: From Possessed Nuns to Chemical and Biological Terrorism Fears,” Br J Psychiatry 180:300-306, 2002; Boss, L. P., “Epidemic hysteria: a review of the published literature,” Epidemiol Rev 19:233 243 (1997)). For instance, during the Persian Gulf War, the first missile attack on Israel by Iraq was widely feared to contain chemical weapons. Although such fears were unfounded, 40% of civilians in the immediate vicinity of the attack reported breathing problems (Bartholomew, R. E., and S. Wessely, “Protean Nature of Mass Sociogenic Illness: From Possessed Nuns to Chemical and Biological Terrorism Fears,” Br J Psychiat 180:300-306 (2002)).

Hence, effective emergency management of a nuclear or radiological event will require two sequential stages: initial rapid identification of exposed individuals from amongst the masses of unexposed, followed by triage of victims to dose-appropriate medical interventions based on accurate biodosimetry. Unfortunately, there is a critical unmet need for the radiation diagnostics required to perform both of these stages of emergency management.

Initial triage (stage 1) is critical to reduce the burden on the healthcare system and conserve precious resources for treatment of individuals acutely at risk. Any diagnostic used for initial triage must be amenable to emergency use on large numbers of panicked people. The sheer number of people to be screened will necessitate ease of use, no need for specialized equipment, little or no training required to administer, reduced (or no) technician time required, and near-immediate results. Detailed biodosimetry (stage 2) is important because moderate exposure to ionizing radiation (IR) (<10 Gy) can be survived with dose-appropriate medical intervention (Dainiak, N., and R. C. Ricks, “The Evolving Role of Haematopoietic Cell Transplantation in Radiation Injury: Potentials and Limitations,” BJR Suppl 27:169-174 (2005); Waselenko, J. K., et al., “Medical Management of the Acute Radiation Syndrome: Recommendations of the Strategic National Stockpile Radiation Working Group,” Ann Intern Med 140:1037-1051 (2004); “Summaries for Patients. Medical Management of the Acute Radiation Syndrome: Recommendations of the Strategic National Stockpile Radiation Working Group,” Ann Intern Med 140, 2004, p. 151; Dainiak, N., “Hematologic Consequences of Exposure to Ionizing Radiation,” Exp Hematol 30:513-528 (2002); Alpen, E., “Radiation Biophysics,” 2nd ed., Academic Press, San Diego, Cal., 1998). For example, the Strategic National Stockpile (SNS) Radiation Working Group recommends dividing patients into four major treatment categories: normal care (1-3 Gy), critical care (3-5 Gy), intensive care (5-10 Gy), and expectant care (≧10 Gy) (Waselenko, J. K., et al., “Medical Management of the Acute Radiation Syndrome: Recommendations of the Strategic National Stockpile Radiation Working Group,” Ann Intern Med 140:1037-1051 (2004)). A more recent report recommended specific therapeutic guidelines for antibiotics, cytokines, and transplantation in the event of radiologic event (Weisdorf, D., et al., “Acute Radiation Injury: Contingency Planning for Triage, Supportive Care, and Transplantation,” Biol Blood Marrow Transplant 12:672-682 (2006)); patients exposed to >2 Gy would receive antibiotics, and patients exposed to >3 Gy would receive cytokine support. Transplantation would be reserved for patients exposed to 7-10 Gy (Weisdorf, D., et al., “Acute Radiation Injury: Contingency Planning for Triage, Supportive Care, and Transplantation,” Biol Blood Marrow Transplant 12:672-682 (2006)).

Developing procedures for triage and medical management of exposed individuals is complicated by uncertainties concerning the nature of exposure. For example, the severity of injury to individual organs varies with radiation dose rate, quality of radiation (low versus high linear energy transfer, LET), heterogeneity of exposure (partial versus total body), source of exposure (external radiation versus internal contamination), and is likely modulated by the host's inherent sensitivity. Physical dosimetry would be essentially impossible. Only biodosimetry has the potential to quantify individual exposures for guiding dose-appropriate medical intervention.

Assays presently available for biodosimetric determinations suffer from inaccuracy, high expense, and/or long analysis times, and many are not amenable to point-of-care (POC) in emergency conditions. The “gold standard” for radiation biodosimetry is cytogenetic analysis (chromosome aberrations, micronuclei) of peripheral blood lymphocytes (Waselenko, J. K., et al., “Medical Management of the Acute Radiation Syndrome Recommendations of the Strategic National Stockpile Radiation Working Group,” Ann Intern Med 140:1037-1051 (2004)). This provides a highly accurate measure of exposure that has the potential to distinguish exposures to different LETs, and can be used when there are partial body exposures, thanks to the continual mixing of lymphocytes in blood. Its limitations are that assays take 2-3 days and require culture of cells in laboratories; the assays are not portable. Another parameter, lymphocyte depletion kinetics, requires multiple measurements over many days and leads to dose estimations that are too late for most intervention therapies (Grayson, J. M., et al., “Differential Sensitivity of Naive and Memory CD8+ T Cells to Apoptosis in vivo,” J Immunol 169:3760-3770 (2002); Cui, Y. F., “Apoptosis of Circulating Lymphocytes Induced by Whole Body Gamma-Irradiation and Its Mechanism,” J Environ Pathol Toxicol Oncol 18:185-189 (1999); Grace, M. B., et al., “Use of a Centrifuge-Based Automated Blood Cell Counter for Radiation Dose Assessment,” Mil Med 171:908-912 (2006); Goans, R. E., et al., “Early Dose Assessment Following Severe Radiation Accidents,” Health Phys 72:513-518 (1997); Parker, D. D., and J. C. Parker, “Estimating Radiation Dose From Time to Emesis and Lymphocyte Depletion,” Health Phys 93:701-704 (2007)). Finally, estimating exposure using time to onset of vomiting is highly inaccurate given the variability of the prodromal syndrome (Parker, D. D., and J. C. Parker, “Estimating Radiation Dose From Time to Emesis and Lymphocyte Depletion,” Health Phys 93:701-704 (2007); Demidenko, E., et al., “Radiation Dose Prediction Using Data on Time to Emesis in the Case of Nuclear Terrorism,” Radiat Res 171:310-309 (2009)). This critical unmet need for adequate radiation-exposure biomarkers has stimulated searches for sensitive markers of exposure including gene expression profiles (Amundson, S. A., et al., “Fluorescent cDNA Microarray Hybridization Reveals Complexity and Heterogeneity of Cellular Genotoxic Stress Responses,” Oncogene 18:3666-3672 (1999); Amundson, S. A., et al., “Identification of Potential mRNA Biomarkers in Peripheral Blood Lymphocytes for Human Exposure to Ionizing Radiation,” Radiation Research 154:342 (2000); Amundson, S. A., et al., “Biological Indicators for the Identification of Ionizing Radiation Exposure in Humans,” Expert Rev Mol Diagn 1:211-219 (2001); Amundson, S. A., and A. J. Formace, Jr., “Gene Expression Profiles for Monitoring Radiation Exposure,” Radiat Prot Dosimetry 97:11-16 (2001); Amundson, S. A., et al., “Induction of Gene Expression as a Monitor of Exposure to Ionizing Radiation,” Radiat Res 156:657-661 (2001); Amundson, S. A., et al., “Human in vivo Radiation-Induced Biomarkers: Gene Expression Changes in Radiotherapy Patients,” Cancer Res 64:6368-6371 (2004); Akerman, G. S., et al., “Alterations in Gene Expression Profiles and the DNA-Damage Response in Ionizing Radiation-Exposed TK6 Cells,” Environ Mol Mutagen 45:188-205 (2005)), protein profiles (Marchetti, F., et al., “Candidate Protein Biodosimeters of Human Exposure to Ionizing Radiation,” Int J Radiat Biol 82:605-639 (2006); Ivey, R. G., et al., “Antibody-Based Screen for Ionizing Radiation-Dependent Changes in the Mammalian Proteome for Use in Biodosimetry,” Radiat Res 171:549-546 (2009); Desai, N., et al., “Simultaneous Measurement of Multiple Radiation-Induced Protein Expression Profiles Using the Luminex™ System,” Adv Space Res 34:1362-1367 (2004)), urine metabolomic profiles (Tyburski, J. B., et al., “Radiation Metabolomics. 2. Dose- and Time-Dependent Urinary Excretion of Deaminated Purines and Pyrimidines After Sublethal Gamma-Radiation Exposure in Mice,” Radiat Res 172:42-57 (2009); Tyburski, J. B., et al., Radiation Metabolomics. 1. Identification of Minimally Invasive Urine Biomarkers for Gamma-Radiation Exposure in Mice,” Radiat Res 170:1-14 (2008)), and changes in tooth enamel (Swartz, H. M., et al., “In vivo EPR For Dosimetry,” Radiat Meas 42:1075-1084 (2007)), as well as work towards automating chromosomal assays to enable high throughput measurements.

Therefore, a need exists for reagents and methods for use in assessing the exposure to ionizing radiation.

### SUMMARY

In one aspect, the invention provides a method for assessing the exposure of a subject to ionizing radiation comprising measuring the presence or amount of Smc1 protein phosphorylated at least at one of serine 957 or serine 966 in a biological sample obtained from the subject. The method comprises (i) contacting the biological sample with a capture reagent that specifically binds to a first epitope on the Smc1 protein; (ii) contacting the biological sample with at least one detection reagent that specifically binds to phosphorylated serine 957 or phosphorylated serine 966 with reference to human Smc1 protein (SEQ ID NO:6); and (iii) determining the presence or amount of the bound detection reagent, wherein an increased amount of bound detection reagent in comparison to a reference standard, or an amount of bound detection agent above a reference threshold value indicates that the subject was exposed to ionizing radiation.

In another aspect, the invention provides a kit for detecting the presence or amount of Smc1 protein phosphorylated at one of serine 957 or serine 966 in a biological sample. The kit comprises: (i) a capture reagent that specifically binds to a first epitope on the Smc1 protein; and (ii) at least one detection reagent that specifically binds to a second epitope comprising phosphorylated serine 957 or phosphorylated serine 966 with reference to human Smc1 protein.

In another aspect, the invention provides a device for point of care detection of exposure to ionizing radiation, wherein the device indicates the presence of Smc1 protein phosphorylated at serine 957 or serine 966 in a biological fluid sample. The device comprises: (i) a sample receiving zone adapted to receive a biological fluid sample, (ii) an analyte detection region comprising a porous material which conducts lateral flow of the fluid sample, wherein the analyte detection region comprises an immobile indicator capture reagent that specifically binds to a first epitope on the Smc1 protein; and (iii) a detection labeling reagent zone comprising a first mobile detection labeling reagent that specifically binds to phosphorylated serine 957 or phosphorylated serine 966 with reference to the Smc1 protein (SEQ ID NO:6), wherein the sample receiving zone is in lateral flow contact with the detection labeling reagent zone and with the analyte detection region.

In another aspect, the invention provides a method for determining the susceptibility of a subject to ionizing radiation exposure. The method according to this aspect of the invention comprises: (a) obtaining one or more biological test sample(s) from a subject; (b) exposing at least a portion of said biological test sample(s) to one or more predetermined dosages of ionizing radiation; and (c) determining the presence or amount of Smc1 protein phosphorylated at least at one of serine 957 or serine 966, with reference to human Smc1 protein (SEQ ID NO:6) in the biological sample(s) exposed to radiation in accordance with step (b), wherein the amount or presence phosphorylated Smc1 protein detected in the biological test sample in comparison to a control or reference standard is indicative of the subject's susceptibility to exposure to ionizing radiation.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A is a panel of Western Blot screens illustrating the presence of several biomarkers in human (Hs) and canine (CO cell lysates after the cells received 0 or 10 Gy ionizing radiation, as described in Example 1;

FIG. 1B is a panel of Western Blots illustrating the levels of phosphorylated and unphosphorylated forms of Smc1 in canine PBMC lysates after in vivo exposure to 0 or 10 Gy total body ionizing radiation, as described in Example 1;

FIG. 2 illustrates the structure of the human Smc1 protein and Smc1 peptides fragments used as immunogenic peptides and synthetic peptide standards, as described in Example 2;

FIG. 3A is a diagrammatic illustration of the use of a synthetic phosphopeptide reference molecule FHC37_FCp, derived from Smc1, in a sandwich ELISA assay format. In the illustrated embodiment, the detection mAb is a bivalent antibody that is conjugated with a detectable agent, indicated by the star symbol, which can be an agent such as a colloidal gold particle; as described in Example 2;

FIG. 3B illustrates the detection of a phosphorylated Smc1 polypeptide in a sandwich ELISA assay format, indicating the relative positions of Capture mAb, the Smc1 protein, and Detection mAb. In the illustrated embodiment, the Detection mAb is a bivalent antibody that is biotinylated (B), and a detectable agent, indicated by the star symbol, is a labeled biotin binding agent, such as streptavidin; as described in Example 2;

FIG. 4 graphically illustrates a standard curve that was generated by plotting the concentration of the synthetic reference phosphopeptide (SEQ ID NO:5) versus OD450, a measure of the formation of a binding complex between the biotinylated detection mAb, the capture mAb, and the synthetic reference phosphopeptide, as described in Example 2;

FIG. 5A graphically illustrates a competition assay where endogenous phosphorylated Smc1 was measured by the phospho-Smc1 (pS957) ELISA in the presence of increasing concentrations of Smc1_F_DP hybrid peptide as a competitor, or a non-specific peptide, as described in Example 2;

FIG. 5B graphically illustrates a competition assay where endogenous phosphorylated Smc1 was measured by the phospho-Smc1 (pS966) ELISA in the presence of increasing concentrations of Smc1_F_CP hybrid peptide as a competitor, or a non-specific peptide, as described in Example 2;

FIG. 5C graphically illustrates a mixing experiment demonstrating the linearity of the phospho-Smc1 (pS957) ELISA, where lysates from cells exposed to 0 or 5 Gy were mixed in several proportions and the levels of phosphorylated Smc1 were measured, as described in Example 2;

FIG. 5D graphically illustrates a mixing experiment demonstrating the linearity of the phospho-Smc1 (pS966) ELISA, where lysates from cells exposed to 0 or 5 Gy were mixed in several proportions and the levels of phosphorylated Smc1 were measured, as described in Example 2;

FIG. 5E graphically illustrates a recovery assay, wherein increasing known amounts of Smc_F_Cp hybrid peptide was added to mock irradiated lysates or dilution buffer and levels of the Smc_F_Cp were measured with the phospho-Smc1 (pS957) ELISA, as described in Example 2;

FIG. 5F graphically illustrates a recovery assay, wherein increasing known amounts of Smc_F_Dp hybrid peptide was added to mock irradiated lysates or dilution buffer and levels of the Smc_F_Dp were measured with the phospho-Smc1 (pS966) ELISA, as described in Example 2;

FIG. 6 graphically illustrates the phospho-Smc1 (p966) concentrations in Lymphoblast Cell Line (LBL) cells, wherein the LBL was divided and exposed to no or increasing doses of ionizing radiation (IR), lysed, and subjected to the phospho-Smc1 (p966) ELISA. The resulting OD450 value for each lysate was converted to a molar concentration by way of the equation of the line generated from the standard reference peptide (illustrated in FIG. 4), as described in Example 2;

FIG. 7A graphically illustrates the detection of dose-dependent induction of phospho-Smc1 (pS957) (and two other markers: Rad17 and P53) in two independent lymphoblastoid cell lines (LBLs), GM10834 and GM07057, measured by ELISA 4 hours after exposure to 0, 2, 4, 7, and 10 Gy ionizing radiation, as described in Example 2;

FIG. 7B graphically illustrates the detection of time-dependent induction of phospho-Smc1 (pS957) (and another marker, P53) in human LBL cells, measured by ELISA before and 2, 4, 8, 12, and 24 hours after exposure to 5 Gy ionizing radiation, as described in Example 2;

FIG. 8A graphically illustrates the detection of dose-dependent induction of phospho-Smc1 (pS957 and pS966) in LBL GM07057, measured by ELISA 2 hours after exposure to 0, 2, 4, 8, and 12 Gy ionizing radiation, as described in Example 2;

FIG. 8B graphically illustrates the detection of time-dependent induction of phospho-Smc1 (pS957 and pS966) in LBL GM07057, measured by ELISA before and 2, 4, 8, 12, 24 and 48 hours after exposure to 5 Gy ionizing radiation, as described in Example 2;

FIG. 9A graphically illustrates the detection of time-dependent induction of phospho-Smc1 (pS957 and pS966) in LBL GM07057, measured by ELISA before and 2, 4, 8, 12, 24 and 48 hours after exposure to 2 Gy ionizing radiation, as described in Example 2;

FIG. 9B is a panel of Western blots that validate the time-dependent induction of phospho-Smc1 (pS957 and pS966) in LBL GM07057, measured using antibodies specific for pan Smc1 (FHC37F), Smc1 pS957 (FHC37 Cp), and Smc1 pS966 (FHC37Dp) before and 2, 8, 12, 24 and 48 hours after exposure to 2 Gy ionizing radiation, as described in Example 2;

FIG. 10 graphically illustrates the IR dose- and time-dependent induction of phospho-Smc1 (pS957 and pS966) in LBL cells that expressed or were deficient in ATM kinase, as described in Example 2;

FIG. 11A graphically illustrates the dose-dependent levels of phospho-Smc1 (pS957 and pS966) in LBL GM010860 after exposure to ionizing radiation with doses ranging from 0.5 Gy to 12 Gy, as described in Example 2;

FIG. 11B graphically illustrates the fold induction of phospho-Smc1 (pS957 and pS966) in LBL GM010860 after exposure to ionizing radiation with doses ranging from 0.5 Gy to 12 Gy, as described in Example 2;

FIG. 12 graphically illustrates the fold induction of phospho-Smc1 (pS957 and pS966) in three murine models after exposure of 10 or 2.75 Gy of total body irradiation, as described in Example 3;

FIG. 13A graphically illustrates the levels of phospho-Smc1 (pS957) in canine peripheral blood mononuclear cells (PBMCs) obtained at increasing time points after total body irradiation of 2 or 10 Gy ionizing radiation, as described in Example 3;

FIG. 13B graphically illustrates the levels of phospho-Smc1 (pS957) in canine PBMCs before and at increasing time points after exposure ex vivo to 2 or 10 Gy ionizing irradiation, as described in Example 3;

FIG. 13C graphically illustrates the levels of phospho-Smc1 (pS957) in activated and cultured canine PBMCs before at increasing time points after exposure in vitro to 2 or 10 Gy ionizing irradiation, as described in Example 3;

FIG. 14A graphically illustrates the levels of phospho-Smc1 (pS957) in canine PBMCs obtained at increasing time points after total body irradiation of 2, 6 or 10 Gy ionizing radiation, applied at 7 cGy/minute, as described in Example 3;

FIG. 14B graphically illustrates the levels of phospho-Smc1 (pS957) in canine PBMCs obtained at increasing time points after total body irradiation of 2, 6 or 10 Gy ionizing radiation, applied at 70 cGy/minute, as described in Example 3;

FIG. 15A graphically illustrates the levels of phospho-Smc1 (pS957) in canine PBMCs determined at increasing time points after exposure ex vivo to 2, 6 or 10 Gy ionizing irradiation, applied at 8.5 cGy/minute, as described in Example 3;

FIG. 15B graphically illustrates the levels of phospho-Smc1 (pS957) in canine PBMCs determined at increasing time points after exposure ex vivo to 2, 6 or 10 Gy ionizing irradiation, applied at 66 cGy/minute, as described in Example 3;

FIG. 16 graphically illustrates the levels of phospho-Smc1 (pS957) in activated and cultured canine PBMCs before at increasing time points after exposure in vitro to 2, 6 or 10 Gy ionizing irradiation, applies at 8.5 cGy/minute, 66 cGy/minute, or 529 cGy/minute, as described in Example 3;

FIG. 17A graphically illustrates the detection of dose-dependent induction of phospho-Smc1 (pS957) and two other markers, Rad17 and P53, in cultured and activated human PBMCs measured by ELISA 4 hours post exposure in vitro to 0, 2, 4, 7, or 10 Gy ionizing radiation, as described in Example 4;

FIG. 17B graphically illustrates the detection of time-dependent induction of phospho-Smc1 (pS957) in cultured and activated human PBMCs measured by ELISA before, 2, 8 or 24 hours after exposure in vitro to 0 or 10 Gy ionizing radiation, as described in Example 4;

FIG. 17C graphically illustrates the fold induction of phospho-Smc1 (pS957) and p53 (pS15) in human PBMCs measured by ELISA before, 2, 8 or 24 hours after exposure ex vivo to 0 or 7 Gy ionizing radiation, or to in vivo exposure to 0 or 7 Gy (cultured PBMC), as described in Example 4;

FIG. 18A graphically illustrates the detection of dose- and time-dependent induction of phospho-Smc1 (pS957 and pS966) in cultured and activated human PBMCs, as measured by ELISA in vitro after increasing doses of ionizing radiation, as described in Example 4;

FIG. 18B graphically illustrates the levels of phospho-Smc1 (pS957 and pS966) in human PBMCs measured by ELISA at 2 hours after exposure ex vivo to 0 or 5 Gy ionizing radiation, as described in Example 4;

FIG. 19A illustrates use of a lateral flow point of care (POC) test device to detect the presence and relative amount of two synthetic phosphopeptide reference molecules derived from Smc1 across 9 folds of dilutions, as described in Example 6;

FIG. 19B illustrates use of a lateral flow point of care (POC) test devices to detect the dose-dependent induction of phospho-Smc1 (pS957) in human LBL cells exposed in vitro to 0, 2, or 10 Gy ionizing radiation at two hours post exposure, as described in Example 6;

FIG. 20A is a chart containing photographs of lateral flow point of care (POC) test strips specific for phospho-Smc1 (pS957) after application of lysates derived from leukocytes isolated from whole blood exposed to 0 or 8 Gy ionizing radiation, with or without a spiked-in hybrid peptide as a positive control, as described in Example 6;

FIG. 20B illustrates lateral flow point of care (POC) test strips specific for phospho-Smc1 (pS957) after application of lysates derived from leukocytes isolated from a 100 μl whole blood exposed to 0 or 8 Gy ionizing radiation, with or without a spiked-in hybrid peptide as a positive control, as described in Example 6;

FIG. 20C illustrates lateral flow point of care (POC) test strips specific for phospho-Smc1 (pS957) after application of lysates derived from leukocytes isolated from a 250 μl whole blood exposed to 0 or 8 Gy ionizing radiation, with or without a spiked-in hybrid peptide as a positive control, as described in Example 6;

FIG. 21 diagrammatically illustrates an exemplary lateral flow assay format, as described in Example 6;

FIG. 22A diagrammatically illustrates the schedule of total body irradiation (TBI) conditioning regimen for human transplantation patients and the corresponding schedule of blood draws to assay levels of phosphor-Smc1 (pS957 and pS966), as described in Example 7;

FIG. 22B graphically illustrates the levels of phospho-Smc1 (pS957 and pS966) in human PBMCs obtained during various time points during the series of TBI exposures as illustrated in FIG. 22A, and described in Example 7;

FIG. 22C graphically illustrates the mean levels of phospho-Smc1 (pS957 and pS966) in human PBMCs obtained at increasing various time points during the series of therapeutic TBI exposures as illustrated in FIG. 22A; the cumulative ionizing radiation exposure for each assay time point is indicated, as described in Example 7;

FIG. 23 graphically illustrates the time-dependent levels of phospho-Smc1 (pS957 and pS966) PBMCs obtained from two human patients that received a single TBI fraction of 2Gy, as described in Example 7;

FIG. 24A graphically illustrates the time-dependent levels of phospho-Smc1 (pS957) illustrated in FIG. 23, wherein levels are illustrated for PBMCs obtained from three human patients that received a single TBI fraction of 2Gy, as described in Example 7;

FIG. 24B graphically illustrates the time-dependent levels of phospho-Smc1 (pS966) illustrated in FIG. 23, wherein levels are illustrated for PBMCs obtained from three human patients that received a single TBI fraction of 2Gy, as described in Example 7;

FIG. 25A graphically illustrates the levels of phospho-Smc1 (pS957) in PBMCs isolated from four human patients receiving a single therapeutic partial body ionizing radiation exposure, as determined before and 2 hours post-exposure, as described in Example 7;

FIG. 25B graphically illustrates the mean levels of phospho-Smc1 (pS957) in PBMCs isolated from human patients receiving therapeutic partial body ionizing radiation exposure, as determined before and 2 hours post-exposure, as described in Example 7;

FIG. 25C graphically illustrates the levels of phospho-Smc1 (pS957) in human PBMCs isolated after partial body exposure to ionizing radiation, as illustrated in FIG. 25A; the graph includes and additional human patient and indicates the dose and target of the therapeutic partial body exposure, as described in Example 7;

FIG. 26 graphically illustrates the levels of phospho-Smc1 (pS957 and pS966) in PBMCs isolated from four human patients before and 2 hours after partial body ionizing radiation exposure, as described in Example 7;

FIG. 27 graphically illustrates the levels of phospho-Smc1 (pS957) in PBMCs obtained from a patient receiving an initial test infusion of ¹³¹Iodine-labeled anti-CD20 antibody of 10 mCi, and a later therapeutic dose of 592mCi; five blood draws were obtained: draw 1 (pre-infusion), draw 2 (3 days post-test infusion), draw 3 (11 days post-test infusion, 1 day pre-therapy infusion), draw 4 (1 day post-therapy infusion), and draw 5 (8 days post infusion), as described in Example 7;

FIG. 28A-C graphically illustrates the assay results for Smc1 pS957, wherein the estimated technical variation (σ), within subject variation (σ_{β}) and between subject variation (σ_{α}) are shown at 2 hours (panel A), 8 hours (panel B) and 24 hours (panel C) after exposure, as described in Example 8; and

FIG. 29A-C graphically illustrates the assay results for Smc1 p966, wherein the estimated technical variation (a), within subject variation (σ_{β}) and between subject variation (σ_{α}) are shown at 2 hours (panel A), 8 hours (panel B) and 24 hours (panel C) after exposure, as described in Example 8.

### DETAILED DESCRIPTION

As used herein, the term “phosphorylation site” refers to an amino acid or amino acid sequence of a natural binding domain or a binding partner which is recognized by a kinase or phosphatase for the purpose of phosphorylation (e.g., phosphorylation on tyrosine, serine or threonine) or dephosphorylation of the polypeptide or a portion thereof.

As used herein, the term “epitope” refers to the chemical structure of the immunogen of interest that is recognized by an immune system, such as peptides or phospho-peptides.

As used herein, the term “affinity reagent” refers to any molecule that has affinity for binding to the target sequence of interest. As used herein, affinity reagent includes one or more of the following: a) aptamers; b) affinity reagents identified through screening phage display, chemical, or yeast libraries; c) any of the classes of immunoglobulin molecules of any species, or any molecules derived therefrom, including whole antibodies and any antigen binding fragment (i.e., “antigen-binding portion”) or single chains thereof. Exemplary antibodies include polyclonal, monoclonal, single chain and recombinant antibodies. The terms “monoclonal antibody” or “monoclonal antibody composition” as used herein refer to a preparation of antibody molecules of single molecular composition. A monoclonal antibody composition displays a single binding specificity and affinity for a particular epitope.

A naturally occurring “antibody” is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

As used herein, the term “antigen-binding portion” of an antibody (or simply “antigen portion”), as used herein, refers to full length or one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include a Fab fragment, a monovalent fragment consisting of the V_{L}, V_{H}, C_{L} and CH1 domains; a F(ab)₂ fragment, a bivalent fragment comprising two Fab fragments linked by a disulfide bridge at the hinge region; a Fd fragment consisting of the V_{H} and CH1 domains; a Fv fragment consisting of the V_{L} and V_{H} domains of a single arm of an antibody; a dAb fragment (Ward et al., Nature 341:544-546, 1989), which consists of a V_{H} domain; and an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., Science 242:423-426, 1988; and Huston et al., Proc. Natl. Acad. Sci. 85:5879-5883, 1988). Such single chain antibodies are also intended to be encompassed within the term “antigen-binding portion” of an antibody. These antibody fragments are obtained using conventional techniques known to those of skill in the art, and the fragments are screened for utility in the same manner as are intact antibodies.

As used herein, the term “anti-peptide affinity reagent” refers to any type of affinity reagent (in the preceding general sense) that binds to a peptide or a phosphopeptide for the purpose of enrichment and/or detection of a polypeptide or phosphorylated polypeptide comprising the peptide from a biological sample or processed sample.

As used herein, an affinity reagent, such as an antibody that “specifically binds to a phosphorylated peptide” is intended to refer to an antibody that binds to a phosphorylated peptide with a K_{D} of 1×10⁻¹ M or less.

The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for affinity reagents such as antibodies can be determined using methods well established in the art. An exemplary method for determining the K_{D} of an affinity reagent is by using surface plasmon resonance, or using a biosensor system such as a Biacore® system.

As used herein, the terms “immunogen” and “antigen” refer to the peptide or protein (or phosphorylated versions thereof) to which an affinity reagent was generated.

As used herein, the term “affinity” refers to the strength of interaction between the affinity reagent and antigen at their interaction sites. Within each interaction site, the affinity reagent interacts through chemical forces with the target at numerous sites; the more interactions, the stronger the affinity.

As used herein, the term “cross-reactivity” refers to an affinity reagent or population of affinity reagents binding to epitopes on other antigens. This can be caused either by imperfect specificity of the affinity reagent or by multiple distinct antigens having identical or very similar epitopes. Cross reactivity is sometimes desirable when one wants general binding to a related group of antigens or when attempting cross-species labeling when the antigen epitope sequence is not highly conserved in evolution.

As used herein, the term “exposure to ionizing radiation” refers to exposure to subatomic particles or electromagnetic waves with sufficient energy to remove electrons from atoms. Examples of ionizing subatomic particles include alpha particles, beta particles and neutrons. Electromagnetic waves with shorter wave lengths (higher frequencies) possess higher energy and are more likely to be ionizing. Examples of high energy, or high frequency, ionizing electromagnetic waves include ultraviolet (UV) rays, x-rays and gamma-rays. Exposure to ionizing radiation is commonly known to cause damage to living tissue, including breaks in DNA molecules.

As used herein, the term “lymphoblast cell line” is used interchangeably with “lympohoblastoid cell line” and “LBL”, and refers to maintained cultures of lymphoblast cells derived from human donors. Illustrative lines used herein include: GM10834, GM07057, G05920, GM10860, GM13819 and GM05126. The LBL number identifies each distinct line, referring to its Coriell Institute (Camden, N.J.) Catalog ID number.

As used herein, the term “procedure to diagnose or treat a medical condition” refers to any medical procedure to assess the presence, progression, or resolution of a medical disease in a subject, or to any medical procedure to cure, facilitate the resolution of, or ameliorate the harmful effects of a medical disease. It is contemplated that any assessment or diagnosis may occur before, during or after medical treatment or therapy. Any medical condition or disease is contemplated, including cancers and non-cancer diseases. As a non-limiting example, a procedure to treat cancer refers to any medically prescribed regimen used to treat the condition of unchecked cell proliferation. Typically, such regimens may include administration of agents that disrupt the cell cycle, for instance, the application of ionizing radiation to the body to cause disruption of the cell cycle in tumor cells. Such ionizing radiation may be applied from an external source to the whole body or to a specific region of the body. Alternatively, a source of ionizing radiation may be administered into the body, typically in a manner that targets the source directly to the cancerous tissue.

As used herein, the term “point of care assay” refers to a medical diagnostic test that can be administered quickly, at the point of patient contact, with minimal effort, and can provide a rapid indication of a diagnosis. Based on the rapid diagnosis, a subject's determined medical needs may be quickly assessed.

As used herein, the term “about” refers to plus or minus ten percent (10%) of the referenced value.

The present invention is based, at least in part, on the discovery by the present inventions that methods, reagents, kits and devices can be generated for carrying out a diagnostic assay for use in assessing the exposure to ionizing radiation in a biological sample of interest. As described in Examples 1-7, the inventors have demonstrated that assays for detection and/or quantitation of phospho-Smc1 (pS957) and phospho-Smc1 (pS966) of the Structural Maintenance of Chromosomes 1 (“Smc1”) are useful for assessing the exposure to ionizing radiation in a biological sample, such as a sample obtained from cultured cells exposed to radiation, or a sample obtained from a mammalian subject exposed to radiation. In preferred embodiments, the kits and devices can be stockpiled and distributed for use under emergency conditions to detect radiation exposure in the event of a real or suspected nuclear or radiological event.

In accordance with the foregoing, in one aspect, the invention provides a method for assessing the exposure to ionizing radiation comprising measuring the presence or amount of Smc1 protein phosphorylated at one of serine 957 or serine 966 in a biological sample, the method comprising: (i) contacting the biological sample with a capture reagent that specifically binds to a first epitope on the Smc1 protein; (ii) contacting the biological sample with at least one detection reagent that specifically binds to phosphorylated serine 957 or phosphorylated serine 966 with reference to human Smc1 protein; and (iii) determining the presence or amount of the bound detection agent, wherein an increased amount of bound detection reagent in comparison to a reference standard, or an amount of bound detection agent above reference threshold value indicates that the source of the biological sample was exposed to ionizing radiation.

The methods and reagents of the invention can be used to detect and/or measure the presence or amount of Smc1 protein phosphorylated at one of serine 957 or serine 966 in any biological sample that contains protein, such as a biological fluid or a biological tissue. Examples of biological fluids include urine, blood, plasma, serum, saliva, semen, stool, sputum, cerebral spinal fluid, tears, mucus, amniotic fluid or the like. Examples of biological tissues include organs, tumors, lymph nodes, arteries and individual cells, including cells grown in culture. The methods and reagents of the invention can also be used to detect and/or measure the presence or amount of Smc1 protein phosphorylated at one of serine 957 or serine 966 in cells derived from the aforesaid biological tissues. In some embodiments, the cells are sloughed off from the tissues and are collected in biological fluid samples, such as the urine, saliva, or in fecal samples.

In accordance with the methods of the invention, at least a portion of the cells in the biological sample are lysed to release the Smc1 protein. The biological sample may be lysed with any suitable lysis reagent such as, for example, RIPA (Cell Signaling Technology, cat#9806), M-PER (Pierce/Thermo Fisher, cat#78503) or Whole Cell Lysis Buffer (150 mM NaCl, 20 mM b-Glycerophosphate, 10 mM NaF, 1 mM EDTA, 0.5% Triton X-100, 0.005% Tween 20, filter sterilized). Lysis buffers typically contain protease and phosphatase inhibitors (e.g. Sigma, cat#. P2850, P5726, and P8340) at a final concentration of 1%. The cell lysate is then contacted with a capture reagent that specifically binds to a first epitope on the Smc1 protein; (ii) contacting the biological sample with at least one detection reagent that specifically binds to phosphorylated serine 957 or phosphorylated serine 966 with reference to human Smc1 protein, as further described herein.

The methods of the invention may be used to assess exposure to ionizing radiation in a biological sample obtained from any mammalian subject, such as a human, dog, cat, mouse, rat, horse, and the like. In some embodiments, the biological sample is assessed for radiation exposure by the method within a time period greater than 30 seconds after potential exposure to ionizing radiation (such as greater than 1 minute, greater than 5 minutes, greater than 10 minutes, greater than 30 minutes, greater than one hour, or greater than 2 hours). In further embodiments, the biological sample is assessed for radiation exposure within 1, 2, 3, 4, or 5 days or more after the potential or suspected exposure to ionizing radiation. The data described herein indicate that the method detects elevated levels of phosphorylated Smc1 protein for several days after exposure. Therefore, in some embodiments the biological sample is assessed for radiation exposure as much as up to 72 hours after the potential or suspected exposure to ionizing radiation. Illustrative sources of potential exposure to ionizing radiation include a nuclear attack, a nuclear accident, or after a diagnostic test or therapeutic treatment (e.g., cancer treatment).

In some embodiments, the method is self-administered by a human subject after a suspected exposure to a source of ionizing radiation. In such embodiments, the self-administered test is designed to provide a binary distinction as to whether the subject was exposed or not exposed, thereby reducing the burden on the healthcare system and conserving precious resources for treatment of individuals acutely at risk. In some embodiments, the step of contacting the biological sample with a detection reagent is carried out on a diagnostic test strip akin to the widely-used, over-the-counter pregnancy test, but using a blood sample obtained by finger prick (as in the widely-used over-the-counter blood glucose test kits). In accordance with such methods, the radiation test kit could be self-administered “in the field” in emergency situations immediately and without sophisticated technology to assess exposure.

In some embodiments, the method is carried out on cells cultured in a laboratory setting. In such embodiments, the method is designed either to provide a binary distinction as to whether the source of the biological sample was exposed or not exposed to radiation, or to quantify the amount of phosphorylated Serine 957 or phosphorylated Serine 966 (with reference to human Smc1 protein) in the cultured cells.

Selection of the First Epitope from the Smc1 Protein for Binding to a Capture Reagent

The first epitope from the full length Smc1 protein is selected for binding to a capture reagent, such as a capture antibody and uniquely corresponds to the Smc1 protein and serves as a recognition sequence for binding to a capture reagent (e.g., with at least detectable selectivity). Immunogenic domains in a protein of interest may be identified using web-based tools to predict antigenic peptide, such as, for example, the method of Kolaskar, A. S., and P. C. Tongaonkar, FEBS Lett 276:172-4 (1990).

The first epitope may be determined in silico and generated in vitro, such as by peptide synthesis, without cloning or purifying the protein it derives from. The first epitope for binding may be selected by performing a comprehensive search of one or more relevant databases using all theoretically possible epitopes of the Smc1 protein with a given length (e.g., from 5 to 25 continuous amino acid residues in length from a protein of interest). This process is preferably carried out computationally using any of the sequence search tools available in the art. For example, to identify a first epitope from a protein of interest having at least 5 continuous amino acid residues in length, immunogenic domains in the protein of interest may be identified using web-based tools to predict antigenic peptide, such as, for example, the method of Kolaskar, A. S., and P. C. Tongaonkar, FEBS Lett 276:172-4 (1990). In some embodiments, the first epitope of Smc1 for binding to a capture reagent is from 5 amino acids in length up to about 150 amino acids in length, such as from 5 to about 25 amino acids in length, such as from 5 to about 75 amino acids in length.

The first epitope may be derived from any portion of the full length Smc1 protein (e.g., human Smc1 protein, GenBank Ref. No. NP_{—}006297.2, incorporated herein by reference, the sequence of which is provided herein as SEQ ID NO:6). In some embodiments, the first epitope is derived from the amino half of the protein of interest (i.e. an amino acid 5′ of the mid point of the Smc1 protein coding sequence). In some embodiments, the first epitope is derived from the carboxy half of the protein (i.e. an amino acid sequence 3′ of the mid point of the Smc1 protein coding sequence). In some embodiments, the first epitope comprises the amino acid sequence “5′ DLTKYPDANPNPNEQ 3′” (SEQ ID NO:1).

A synthetic peptide comprising the amino acid sequence of the first epitope may be used to raise a capture reagent, such an antibody specific for the first epitope (e.g., a capture antibody), as described in Example 2.

Selection of the Second Epitope for Detection of Phosphorylated Serine 957 or Phosphorylated Serine 966 of the Smc1 Protein

The second epitope is selected for binding to a detection reagent, such as a detection antibody that selectively binds to the phosphorylated serine 957 or phosphorylated serine 966 of the Smc1 protein. The second epitope is selected to correspond to the protein of interest and serves as a recognition sequence for binding to a detection reagent (e.g., with at least detectable selectivity).

In some embodiments, the second epitope is an amino acid sequence comprising from 5 to about 150 amino acid residues of the target protein of interest (such as from 5 to about 25 amino acids in length, such as from 5 to about 75 amino acids in length), said second epitope comprising at least one of serine 957 or serine 966.

A synthetic phosphopeptide comprising the amino acid sequence of the second epitope that contains phosphorylated serine 957 or phosphorylated serine 966 of the Smc1 protein may be generated as described in Example 2.

Generation of a Synthetic Hybrid Reference Peptide for Use as a Quantitation Standard

In some embodiments, the method utilizes a synthetic hybrid reference peptide as a quantitation standard. The synthetic hybrid reference peptide comprises (i) the first epitope of Smc1 that is bound by the capture reagent and (ii) a second epitope from the Smc1 protein comprising serine 957 or serine 966, wherein the synthetic reference peptide is capable of simultaneously binding to both the capture reagent and the at least one detection reagent. In some embodiments, the synthetic hybrid reference peptide is a phosphopeptide comprising a phosphorylated amino acid at either serine 957 or serine 966.

In some embodiments, the synthetic reference peptide further comprises an amino acid spacer region from 1 to about 50 amino acid residues between the first and second epitopes. In some embodiments, the phosphorylation site (serine 957 or serine 966) is positioned in the synthetic reference peptide such that at least 1 to 10 amino acid residues separate the first epitope from the phosphorylation site.

Generation of Capture Affinity Reagents

As used herein, the term “capture affinity reagent” includes any affinity reagent which is capable of binding to an Smc1 protein that includes the first epitope, with at least detectable selectivity. In a preferred embodiment, the capture agent is an antibody or a fragment thereof, such as a polyclonal antibody, a monoclonal antibody or fragment thereof, or a single chain antibody or a reagent selected from a displayed library.

In accordance with the methods of the invention, a capture agent is generated that binds to a first epitope on Smc1 protein or a peptide derived from Smc1. Any art-recognized method can be used to generate a capture reagent that specifically binds to the first epitope. For example, a synthetic immunopeptide comprising the first epitope can be generated, either with or without an N-terminal spacer sequence, for example, as described in Example 2. The immunopeptide can be used alone or linked to an immunostimulatory agent and used to immunize a suitable subject (e.g., rabbit, goat, mouse, or other mammal or vertebrate) or to screen a display library (e.g., phage, yeast, aptamer). If a subject is immunized, at the appropriate time after immunization, antibody-producing cells can be obtained from the subject and used to prepare monoclonal antibodies by standard techniques, such as the hybridoma technique originally described by Kohler and Milstein, Nature 256:495-497, 1975, incorporated herein by reference. Once the candidate capture agent antibodies are generated, the candidate antibodies may be screened for affinity to the Smc1 protein to identify the most suitable antibodies for use as a capture reagent.

A plurality of capture agents may be attached to a support having a plurality of discrete regions (features), such as an array or test strip. The capture agent array can be produced on any suitable solid surface, including silicon, plastic, glass, polymer, such as cellulose, polyacrylamide, nylon, polystyrene, polyvinyl chloride or polypropylene, ceramic, photoresist or rubber surface. Preferably, the silicon surface is a silicon dioxide or a silicon nitride surface. Also preferably, the array is made in a chip format. The solid surfaces may be in the form of tubes, beads, discs, silicon chips, microplates, polyvinylidene difluoride (PVDF) membrane, nitrocellulose membrane, nylon membrane, other porous membrane, non-porous membrane, e.g., plastic, polymer, perspex, silicon, amongst others, a plurality of polymeric pins, or a plurality of microtitre wells, or any other surface suitable for immobilizing proteins and/or conducting an immunoassay or other binding assay.

Generation of Detection Affinity Reagents

A detection affinity reagent, such as an antibody, is generated that specifically binds to a second epitope on the Smc1 protein comprising serine 957 or serine 966. In some embodiments, the detection affinity reagent is an anti-phospho-antibody that specifically binds to a second epitope comprising phosphorylated serine 957 or phosphorylated serine 966.

Any art-recognized method can be used to generate a detection reagent that specifically binds to the second epitope, either in the modified or unmodified form. For example, a synthetic immunopeptide comprising the first epitope can be generated, either with or without an N-terminal spacer sequence, for example as described in Example 2. The immunopeptide can be used alone or linked to an immunostimulatory agent and used to immunize a suitable subject (e.g., rabbit, goat, mouse, or other mammal or vertebrate), or to screen a display library (e.g., phase, yeast, aptamer). If a subject is immunized, at the appropriate time after immunization, antibody-producing cells can be obtained from the subject and used to prepare monoclonal antibodies by standard techniques, such as the hybridoma technique originally described by Kohler and Milstein, Nature 256:495-497, 1975. Once the candidate detection antibodies are generated, the candidate antibodies may be screened for affinity to the target protein to identify the most suitable antibodies for use as a detection reagent.

In some embodiments, the detection agent, such as an anti-phospho-antibody, is labeled with a detectable moiety such as an enzyme, a fluorescent label, a stainable dye, a chemiluminescent compound, a colloidal particle, a radioactive isotope, a near-infrared dye, a DNA dendrimer, a water-soluble quantum dot, a latex bead, a selenium particle, or a europium nanoparticle.

In one embodiment, said detection agent is a labeled antibody specific for phosphorylated serine. In one embodiment, said detection antibody is labeled by an enzyme or a fluorescent group. In one embodiment, said enzyme is HRP (horse radish peroxidase). In one embodiment, said detection agent is labeled with a fluorescent dye that specifically stains phosphoamino acids. In one embodiment, said fluorescent dye is Pro-Q Diamond dye. In one embodiment, the detection agent is labeled with biotin, wherein colorimetric detection is indicative of binding to an avidin-HRP conjugate.

In some embodiments, Enzyme-Linked Immunosorbent Assay (ELISA) is used for detection of a protein that interacts with a capture agent. In an ELISA, the indicator molecule is covalently coupled to an enzyme and may be quantified by determining with a spectrophotometer the initial rate at which the enzyme converts a clear substrate to a correlated product. Methods for performing ELISA are well known in the art and described in, for example, Perlmann, H., and P. Perlmann, “Enzyme-Linked Immunosorbent Assay,” Cell Biology: A Laboratory Handbook, Academic Press, Inc., San Diego, Calif., pp. 322-328, 1994; Crowther, J. R., “Methods in Molecular Biology, Vol. 42-ELISA: Theory and Practice,” Humana Press, Totowa, N.J., 1995; and Harlow, E., and D. Lane, “Antibodies: A Laboratory Manual,” Cold Spring Harbor Laboratory Press, Cold Spring Harbor, N.Y., pp. 553-612, 1988, the contents of each of which are incorporated by reference. Sandwich (capture) ELISA may also be used to detect a protein that interacts with a capture affinity reagent (e.g., capture antibody) and a detection affinity reagent (e.g., detection antibody). Sandwich ELISAs for the quantitation of proteins of interest are especially valuable when the concentration of the protein in the sample is low and/or the protein of interest is present in a sample that contains high concentrations of contaminating proteins.

Preparation of Synthetic Hybrid Peptides:

Synthetic hybrid peptides can be prepared by classical methods known in the art, for example, by using standard solid phase techniques. The standard methods include exclusive solid phase synthesis, partial solid phase synthesis methods, fragment condensation, and classical solution synthesis. Solid phase peptide synthesis procedures are well known in the art and further described by John Morrow Stewart, Solid Phase Peptide Synthesis (2nd Ed. Pierce Chemical Company, 1984). Synthetic peptides can be purified by preparative high performance liquid chromatography and the composition of which can be confirmed via amino acid sequencing. Once the capture affinity reagent and the detection affinity reagent are available, a standard curve is generated using the hybrid reference peptide standard, and the concentration of the target protein in any given sample can be readily determined in an assay using the reagents described herein, for example in an ELISA assay.

For example, as described in Example 2, ELISA assays were developed to detect phosphorylation in the target protein Smc1 using the methods described herein. Generation of an ELISA assay is well known in the art and requires the following parameters: (1) binding of a capture antibody to a solid surface support (e.g., a 96 well plate); (2) a biospecimen; (3) a quantification standard; (4) a labeled detection antibody (e.g., biotinylated); and (5) detection reagents, such as an enzyme-avidin conjugated reagent and a colorimetric substrate.

Determining the Presence or Amount of the Bound Detection Agent

In accordance with an embodiment of the methods of the invention, an increased amount of bound detection reagent in comparison to a reference standard, or an amount of bound detection agent greater than a reference standard or a threshold value, indicates that the subject was exposed to ionizing radiation. In some embodiments, this would involve comparing the amount of bound detection reagent in the test sample to the amount of bound detection reagent in a non-exposed reference control sample. Use of the reagents described herein are capable of detecting exposures to ionizing radiation doses as low as 0.5 Gy. Therefore, in some embodiments, the methods detect exposure to ionizing radiation of 0.5, 1, 2, 4, 5, 6, 7, 8, 8, 10 or more Gy.

In some embodiments, the method of the invention may be used to divide patients into four major treatment categories: normal care (0.5-3 Gy), critical care (3-5 Gy), intensive care (5-10 Gy), and expectant care (>10 Gy) (Waselenko, J. K., et al., “Medical Management of the Acute Radiation Syndrome: Recommendations of the Strategic National Stockpile Radiation Working Group,” Ann Intern Med 140:1037-1051 (2004)). The patients could then be treated according to the assessed exposure, for example, as described in a recent report, specific therapeutic guidelines have been recommended for antibiotics, cytokines, and transplantation in the event of radiologic event (Weisdorf, D., et al., “Acute Radiation Injury: Contingency Planning for Triage, Supportive Care, and Transplantation,” Biol Blood Marrow Transplant 12:672-682 (2006)); patients exposed to >2 Gy would receive antibiotics, and patients exposed to >3 Gy would receive cytokine support. Transplantation would be reserved for patients exposed to 7-10 Gy (Weisdorf, D., et al., “Acute Radiation Injury: Contingency Planning for Triage, Supportive Care, and Transplantation,” Biol Blood Marrow Transplant 12:672-682 (2006)).

In another aspect, the invention provides a method for determining the susceptibility of a subject to ionizing radiation exposure. The method according to this aspect of the invention comprises: (a) obtaining one or more biological test sample(s) from a subject; (b) exposing at least a portion of said biological test sample(s) to one or more predetermined dosages of ionizing radiation; and (c) determining the presence or amount of Smc1 protein phosphorylated at least at one of serine 957 or serine 966, with reference to human Smc1 protein (SEQ ID NO:6) in the biological sample(s) exposed to radiation in accordance with step (b), wherein the amount or presence phosphorylated Smc1 protein detected in the biological test sample in comparison to a control or reference standard is indicative of the subject's susceptibility to exposure to ionizing radiation.

In some embodiments, the biological test sample is obtained from the subject prior to exposure to ionizing radiation. In some embodiments, the biological test sample is obtained from the subject during a course of radiation treatment.

In some embodiments of the method, the subject is a mammalian subject, such as a human subject. In some embodiments, the human subject is not afflicted with the genetic disorder ataxia telangiectasia. In some embodiments, the subject is a cancer patient and the method is carried out prior to therapeutic treatment of the subject (e.g. radiation therapy, chemotherapy, or other therapeutic treatment). In some embodiments, the subject is a cancer patient and the method is carried out prior to therapeutic treatment of the subject with ionizing radiation in order to determine the appropriate course of treatment in accordance with the subject's susceptibility (i.e. inherent radiosensitivity) to high-grade toxicity from exposure to ionizing radiation.

In some embodiments, the biological sample obtained from the subject is selected from the group consisting of cultured cells, tissue, blood, plasma, serum, urine, saliva, semen, stool, sputum, cerebral spinal fluid, tears, and mucus, or cells derived therefrom (i.e. primary cells). In some embodiments, the biological sample is a blood sample.

In some embodiments, step (c) of the method comprises (i) contacting the biological sample of (b) with a capture reagent that specifically binds to a first epitope on the Smc1 protein; (ii) contacting the biological sample according to (i) with at least one detection reagent that specifically binds to phosphorylated serine 957 or phosphorylated serine 966; and (iii) determining the presence or amount of the bound detection reagent, in accordance with the methods described herein. In some embodiments, the step of determining the presence or amount of Smc1 protein phosphorylated at least at one of serine 957 or serine 966 is carried out within 15 minutes to twenty-four hours (such as within 30 minutes to 24 hours, or within one hour to 24 hours, or within 15 minutes to 8 hours, or within 2 hours to 8 hours) after the biological sample is exposed to ionizing radiation.

In some embodiments of the method in accordance with this aspect of the invention, the reference standard is derived from one or more healthy subjects known to not be afflicted with the genetic disorder ataxia telangiectasia (AT), wherein a decrease in the presence or amount of Smc1 phosphorylation detected in the test sample as compared to the reference standard indicates that the subject has an increased susceptibility to ionizing radiation exposure. In some embodiments, the healthy subjects not afflicted with AT are cancer survivors that have previously undergone radiation therapy.

In some embodiments of the method, the reference standard is derived from one or more subjects known to be afflicted with the genetic disorder ataxia telangiectasia (AT), wherein an increase in the presence or amount of Smc1 phosphorylation detected in the test sample as compared to the reference standard indicates that the subject does not have an increased susceptibility to ionizing radiation exposure.

In another aspect of the invention, a kit is provided for detecting the presence or amount of Smc1 protein phosphorylated at one of serine 957 or serine 966 in a biological sample. The kit comprises (i) a capture reagent that specifically binds to a first epitope on the Smc1 protein; and (ii) at least one detection reagent that specifically binds to a second epitope comprising phosphorylated serine 957 or phosphorylated serine 966 with reference to human Smc1 protein. In some embodiments, the kit further comprises a reference standard, such as a recombinant Smc1 protein, or polypeptide derived therefrom, or a synthetic peptide for use as a positive or negative control. In some embodiments, the reference standard is a synthetic hybrid reference peptide comprising the first epitope and the second epitope, wherein the synthetic hybrid reference peptide is capable of simultaneously binding to both the capture reagent and the at least one detection reagent. The capture reagents, detection reagents and synthetic hybrid reference peptides may be generated as described herein.

In some embodiments, at least one of the capture reagent or the detection reagent is a polyclonal antibody, a monoclonal antibody or a fragment thereof. In some embodiments, the capture reagent and the detection reagents are monoclonal antibodies, or fragments thereof. In some embodiments, the kit further comprises reagents for conducting an immunoassay, such as an ELISA assay. In further embodiments, the kit comprises a microplate or microtiter plate, wherein at least one of said capture and/or detection monoclonal antibodies is bound to the microplate or microtiter plate in a format suitable for an Enzyme-Linked Immunosorbent Assay (ELISA), such as an ELISA assay format typically used in a hospital laboratory setting. If lateral flow test strips are to be used to conduct the immunoassay, then the antibodies within the kit, and optionally the synthetic hybrid reference standards, will be embedded in the lateral flow test strips.

Point of Care (POC) Testing

In another embodiment, the invention provides a device for point of care detection of exposure to ionizing radiation, wherein the device indicates the presence of Smc1 protein phosphorylated at serine 957 or serine 966 in a biological fluid sample, the device comprising: (i) a sample receiving zone adapted to receive a biological fluid sample, (ii) an analyte detection region comprising a porous material which conducts lateral flow of said liquid sample, wherein said analyte detection region comprises an immobile indicator capture reagent that specifically binds to a first epitope on the Smc1 protein; and (iii) a detection labeling reagent zone comprising a first mobile detection labeling reagent that specifically binds to phosphorylated serine 957 or phosphorylated serine 966 of the Smc1 protein, wherein the sample receiving zone is in lateral flow contact with the analyte detection region.

Rapid, manual immunoassays are used in POC testing, such as the over-the-counter pregnancy test. Two types of rapid, manual assays have been developed: lateral-flow and flow-through. Lateral-flow assays are by far the preferable construct, since these devices can be stored at elevated temperatures and require minimal hands-on manipulation—a single step is all that is needed to run the tests.

An exemplary lateral flow assay format is illustrated in FIG. 21. As shown in FIG. 21, the strip comprises a rectangular strip of a solid substrate. The substrate is preferably porous and permits lateral flow of liquid samples through capillary action within the substrate. The sample is added in a sample receiving zone located in a region in the lower end of the strip (Region A). The regions of a typical lateral flow assay strip are in flow contact, or fluid communication, with each other, thus permitting the lateral flow of liquid from one region to another along the length of the strip (indicated by the arrow).

In operation a biological fluid flows laterally from sample receiving zone through an interceding region of variable size (Region B), to a test zone. The test zone comprises an immobile indicator capture reagent that specifically binds to an analyte of interest. Preferably, the capture reagent binds to a first epitope of the analyte of interest that is distinct from the epitope or region bound by a mobile detection reagent. An analyte conjugated to a detection reagent at the second epitope migrates by capillary action through the membrane in a chromatographic fashion. If analyte is present in sufficient concentration, the conjugate—analyte complex binds to the antibody bound to the substrate, forming a visually detectable colored line on the membrane. The test is read visually minutes after sample addition. In one embodiment, the immobile capture reagent, is an antibody, or fragment thereof, that specifically binds to the analyte of interest. For example, the capture reagent can be an antibody that specifically binds to a region on the Smc1 protein distinct from the regions containing serine 957 or serine 966 (with reference to the human sequence, SEQ ID NO:6).

An optional second region is located between the sample receiving zone and the test zone (i.e. analyte detection region) and can serve as a detection labeling reagent zone. The detection labeling reagent zone can contain reagents meant to interact with the biological sample to facilitate the specific detection of the analyte of interest. In some embodiments, the detection labeling reagent zone can contain a mobile detection labeling reagent, such as an antibody, or fragment thereof, that specifically binds to the analyte of interest. Preferably, the detection reagent binds to the analyte of interest at a epitope distinct from the first epitope recognized by the capture reagent. For example, in one embodiment, the mobile detection labeling reagent can be an antibody that specifically binds to phosphorylated serine 957 or phosphorylated 966 of the Smc1 protein, with reference to the human sequence, SEQ ID NO:6). The reagent is mobile in the sense that upon binding to the analyte of interest, the reagent moves with analyte horizontally within the strip towards the test zone, or analyte detection region. In another embodiment, the detection labeling reagent zone contains additional reagents to facilitate detection of the analyte of interest, such as lysis buffers to lyse intact cells present within the biological fluid.

In the embodiments including the optional second region, the biological fluid sample, such as a drop of blood, can be applied directly to the sample receiving zone for detection of intracellular analytes therein. The sample fluid rehydrates the detection reagent, such as dried, colloidal-gold conjugate, and any other facilitating reagents. If analyte is present in the sample, it reacts with the detection reagent. Detection reagent-bound analyte migrates laterally by capillary action through a membrane in a chromatographic fashion. If analyte is present in sufficient concentration, the conjugate-analyte complex binds to the immobilized capture reagent in the test zone, forming a visually detectable colored line on the membrane.

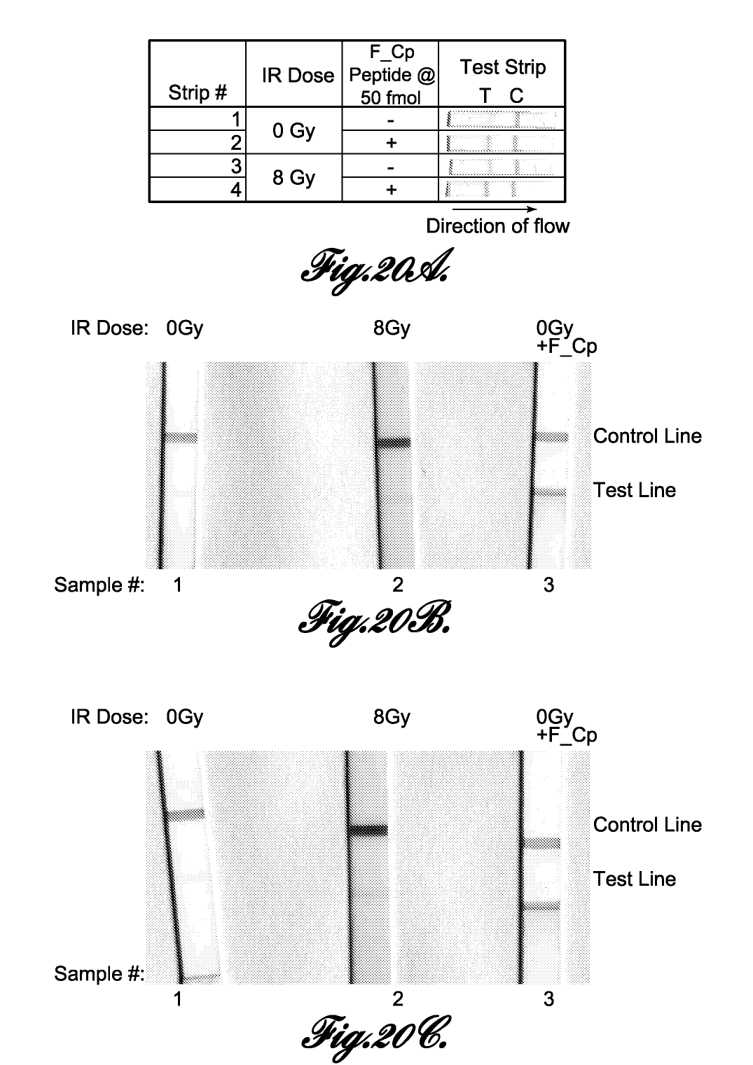

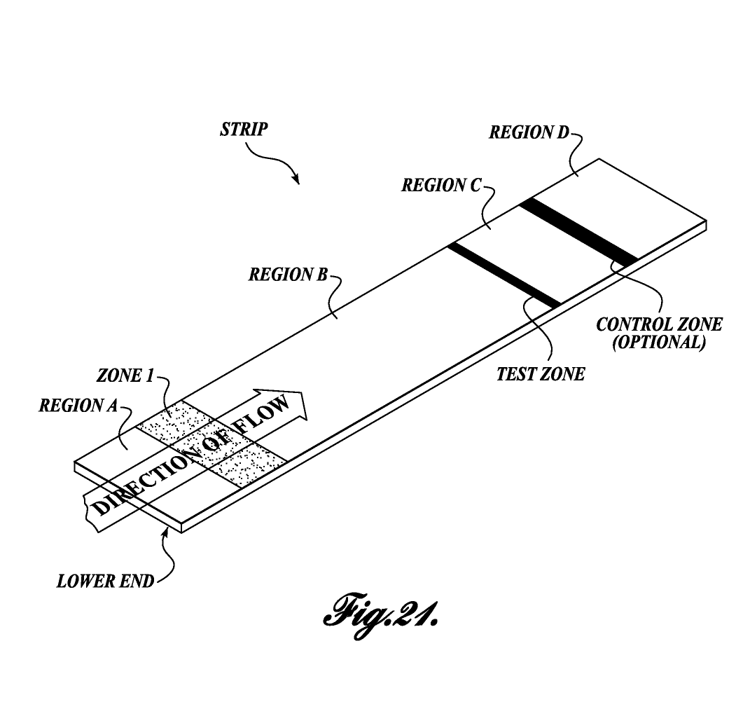

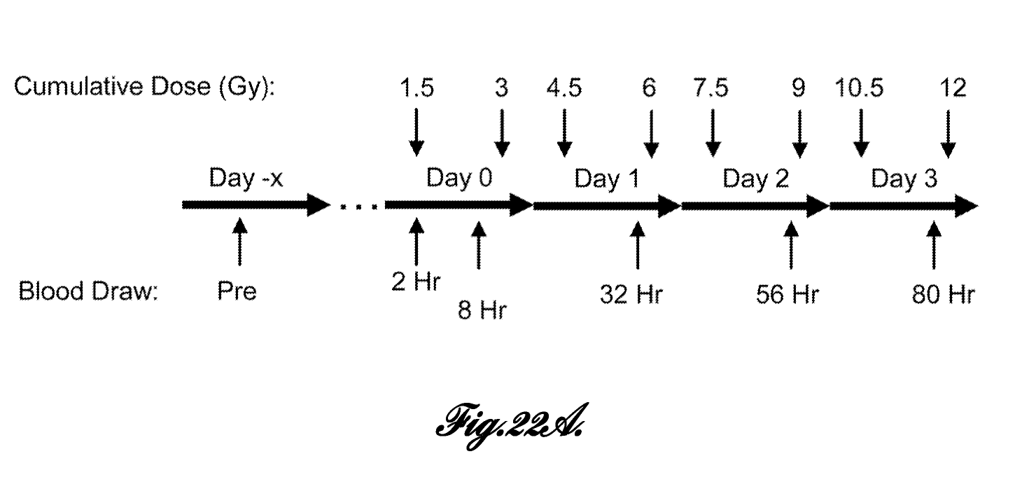

For example, shown in FIGS. 19-20, a biological sample is added to an absorbent pad containing a colloidal gold antibody conjugate (detection reagent). If the analyte (e.g., phosphorylated Smc1) is present in the sample, it reacts with the detection reagent. Detection reagent-bound analyte migrates by capillary action through the membrane in a chromatographic fashion. If the analyte (phosphorylated Smc1) is present in sufficient concentration, the detection reagent-analyte complex binds to the antibody-coated membrane, forming a visually detectable colored line on the membrane.

In some embodiments, the detection labeling reagent and other reagents, are applied to the biological fluid sample before the sample is applied to the solid substrate. For instance, cells may be isolated from a biological sample, lysed, and mixed with a detection reagent. For example, leukocytes may be isolated from blood sample using CD45 Dynabeads, as described in Example 7. The mixture may be subsequently applied to the sample receiving zone.

In some embodiments, an optional control zone is present at a distinct location on the strip from the test zone. The control zone contains immobilized capture reagents that specifically bind to detection reagents that are otherwise unbound to the analyte of interest. For example, as described herein, the control zone contains rabbit antibodies that specifically bind human IgG regions. This control zone serves as a positive control indicator that shows when biological sample containing the detection reagents have successfully moved laterally through the strip. Thus, the control zone is preferably located at a position beyond the test zone in relation to the direction of flow. This embodiment is illustrated in FIG. 19

Further optional features of typical test strips are known in the art, and include absorbent pads at the extreme upper end of the strip to act as a liquid “sink” to facilitate the continued lateral flow movement of the biological fluid sample along the length of the strip.

Many rapid, manual, lateral-flow tests use cellulose-membrane or nylon solid surfaces. Because these have significantly greater surface areas than the wells, tubes, or macro-plastic beads used in the conventional ELISA, up to a hundred times more capture antibody or antigen can be immobilized. Combined with the inherent property of membranes to channel analytes into close proximity with the coated solid-phase, reaction rates occur significantly faster than in ELISA. Additionally, detectable moieties such as colloidal gold nanoparticles of diameters between 30 and 70 nanometers have the advantage of a mobile, liquid-phase that also brings the reactants into close proximity, thereby increasing reaction rate. Since reaction of analyte with solid-phase is usually complete after a few minutes, high degrees of precision and reproducibility are realized.

In some embodiments, the assay of the invention is carried out in the format of a test device that comprises a test strip of rectangular or square dimensions made of a vinyl, polypropylene, or other pliable or non-pliable plastic laminate to serve as a backing to hold in place other test components that are on an adhesive bond on the backing. If the membrane is detecting antigen as the analyte, the surface may be impregnated with antibody or ligand reactive with the antigen. This construct is also known as “sandwich” type rapid assays, since the analyte being detected is captured (sandwiched in between) by both the immuno-conjugate and the membrane surface.

In some embodiments, the assay of the invention is carried out in the format of a test device comprising a fibrous membrane, such as, for example, glass, polyester, cotton, or spun polyethylene, in contact with a membrane containing ligand and bound to densely colored particles such as latex, gold, silver, selenium, carbon, and the like. The bound ligand is complementary to the assay being constructed and reacts with the analyte being detected. The coated colored particles are often described as an immuno-conjugate. Sufficient molecules of ligand are coated onto the surface of the colored particles so that when a positive reaction does occur, the discreet, striped, or spotted zones on the membrane surface are visible to the naked eye. A sample negative for the ligand being detected may leave a white zone in a sandwich type immuno-assay. The colored particles may be dried down onto the fibrous pad or membrane and placed at the dorsal end (at the opposite end of the absorbent pad or membrane) of the membrane. Release agents may be contained in the dried down colored particles to facilitate re-hydration of the particles, allowing them to react with the analyte being detected.

In some embodiments, the assay of the invention is carried out in the format of a test device comprising a fibrous sample receiving pad or membrane, such as glass, polyester, cotton, or spun polyethylene, that is partially in contact with the immuno-conjugate and serves as a reservoir for absorbing and releasing sample. The sample may contain chemicals to facilitate reactive qualities of the assay. The sample may be any biological fluid (bio-fluid) such as tissue extracts, blood, serum, plasma, tears, perspiration, urine, or saliva. The sample may also be derived from an environmental extract, plant extract, or microbial enrichment broth. When a sample or diluted sample is applied to the sample receiving pad or membrane, the movement of liquid is chromatographic and unidirectional towards the absorbent pad or membrane. During migration, the sample re-hydrates the colored particles and reacts with ligand bound to the particles.

While various embodiments of the invention herein are described in the context of a capture affinity agent binding to a first epitope on Smc1 protein and a detection affinity agent binding to a second epitope (such as a modified site) on the Smc1 protein, the invention is not intended to be so limited. It will be understood by those of skill in the art that a protein quantification assay in accordance with the claimed invention can also be carried out in reverse, for example the Smc1 phospho-protein may be captured by an anti-phospho antibody and detected with an antibody that specifically binds to any other epitope on the Smc1 protein, and such embodiments are intended to be encompassed by the present invention.

The following examples merely illustrate the best mode now contemplated for practicing the invention, but should not be construed to limit the invention. All literature citations are expressly incorporated by reference.

### Example 1

This Example describes the initial screen to identify changes in the proteome that could serve as markers for use in biodosimetry.

Methods:

An initial screen was conducted to identify changes in the mammalian proteome that could serve as biomarkers for use in biodosimetry to indicate exposure to ionizing radiation. The screen and initial results are described in Ivey et al., Radiation Research 171(5):549-561 (2009), which is incorporated herein by reference in its entirety.

Briefly, to identify proteomic changes potentially useful for biodosimetry, human cells from lymphoblastoid cell lines (LBL) were treated with 10 Gy of ionizing radiation (IR) or were mock-irradiated and then harvested at different times between 0 and 24 hours post-IR. Lymphoblastoid cell lines were selected for the initial screen because large batches of lysate could be harvested for the screen.

Protein lysates were generated from the harvested LBL cells and evaluated by Western blotting using a panel of 301 commercially available antibodies (Ab) targeting 161 unique proteins; 110 of the antibodies indicated IR-responsive changes in the proteome. As a technical quality control (QC) measure, the Western blots were successfully repeated for 42 of the 110. Additionally, 104 of the antibodies were used to confirm IR-responsive changes in the proteome in a completely independent LBL. To ensure that IR-responsive proteomic changes were not an artifact of immortalization of the LBL cells, a subset of 25 antibodies were used in additional Western blots to measure IR-responsive proteins in human peripheral blood mononuclear cells (PBMCs) that were irradiated or mock-irradiated ex vivo and harvested at multiple times between 0 and 24 hours post-IR. Robust radiation-dependent responses with minimal non-specific bands were confirmed in the PBMCs.

Results:

The results of the preliminary antibody screen are summarized as follows: (a) 110 different antibodies that map to 55 unique proteins showed an IR-responsive change; (b) 153 antibodies detected a band at the expected molecular weight but did not detect an IR-dependent change; and (c) 38 antibodies failed to detect the target protein (i.e., no band was observed at the expected molecular weight).

Of the 55 IR-responsive proteins, 29 showed up-regulation and 26 showed down-regulation relative to the non-irradiated sample. Of the 55 proteins, some were previously identified as IR-responsive in a literature review by Marchetti et al., International Journal of Radiation Biology 82:605-39 (2006). The screen identified 14 novel IR-dependent proteomic changes, some of which have been reported to have a transcriptional response to IR (e.g., GSK3A, PHLDA3, PLK1).

For the reported proteomic changes to be useful for dosimetry, they must also occur in circulating blood cells after an in vivo exposure. The canine model was used for an initial test to determine whether radiation-responsive proteins identified after ex vivo radiation could also be detected after in vivo radiation. The canine model of radiation exposure and hematopoiesis is known to be highly predictive of clinical outcomes in humans, and is a valuable substitute for human models because human in vivo data is limited to rare accidental and well-characterized exposures or to therapeutically determined doses and times.

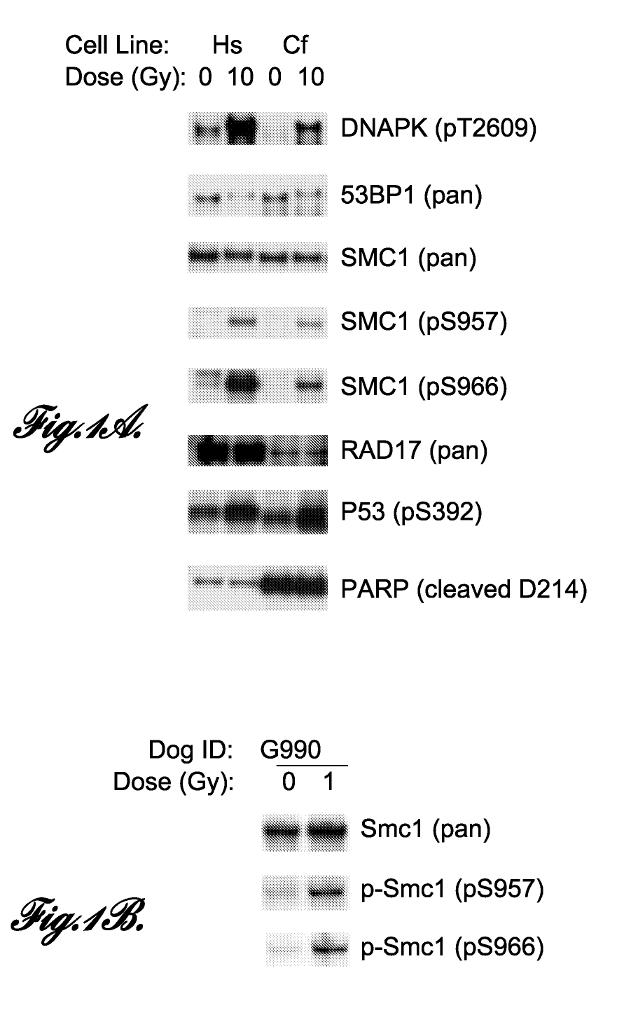

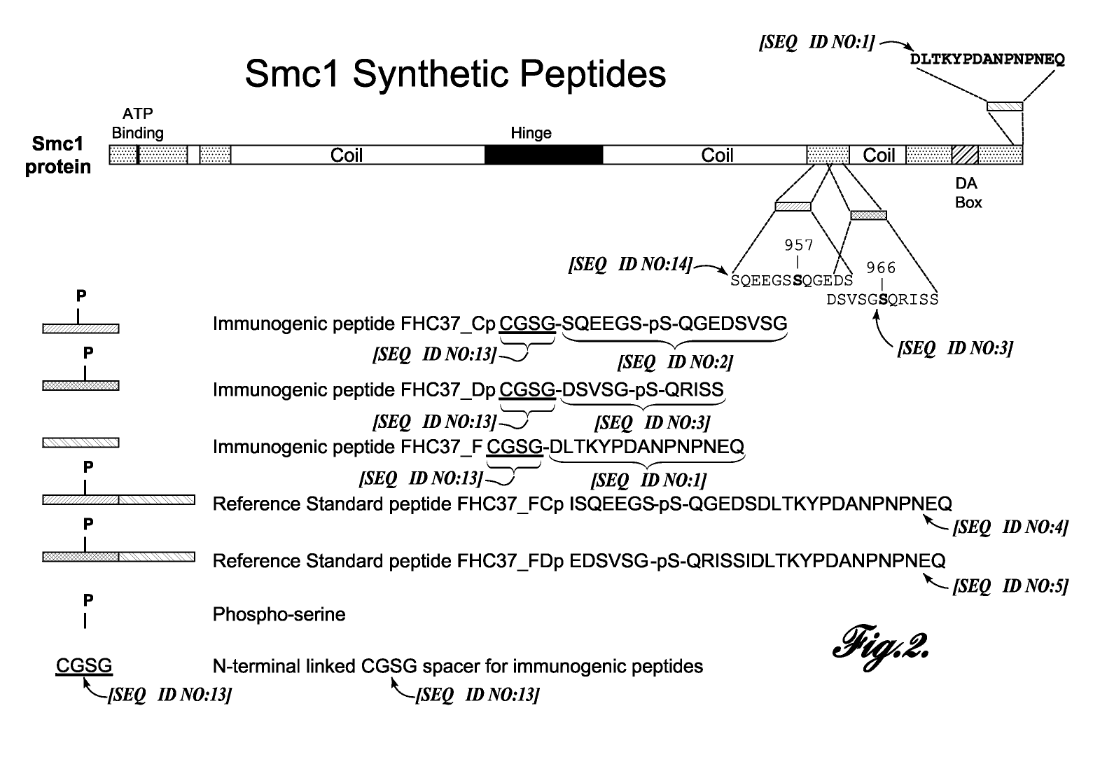

First, a panel of antibodies exhibiting cross-reactivity to the orthologous canine proteins was identified by Western Blot analysis of canine PBMCs irradiated ex vivo. Of 14 antibodies selected from the screen, eight showed cross-reactivity with the orthologous canine proteins (FIG. 1A). Of these eight antibodies that cross-reacted with the canine protein, five antibodies [p-DNAPKT2609, 53BP1 (pan; band shift and reduction), p-Smc1S957, p-Smc1S966 and p-TP53S392] revealed a proteomic change in response to radiation in the canine cells (see FIG. 1A).

Next, animals were exposed to total body irradiation (TBI) to determine whether the radiation-induced phosphorylation of Smc1p observed in the canine cells irradiated ex vivo (shown in FIG. 1A) could also be detected in PBMCs exposed in vivo. Whole blood samples were collected from the animal before TBI and 2 hours after TBI of 1 Gy. PBMCs were isolated, protein lysates were prepared, and the lysates were subjected to Western Blot analysis. Phosphorylation of Smc1 at serine 957 and serine 966 was easily detected in the blood samples collected after TBI compared to samples prepared before TBI, as shown in FIG. 1B.

In summary, a preliminary Western Blot screen using commercially available antibodies successfully identified proteomic changes in cells induced by IR. Among the detectable changes was the induction of phosphorylation of Smc1 at serine 957 and serine 966. The IR-induced phosphorylation of Smc1 was confirmed in canine PBMCs after exposure ex vivo. Additionally, Smc1 phosphorylation is induced in vivo, after total body irradiation. This supports the feasibility of monitoring proteomic response of circulating cells to detect instances of radiation exposure. More specifically, these data indicate that induction of Smc1 phosphorylation is a useful marker for use in biodosimetry.

### Example 2

This Example describes the generation of monoclonal antibodies against phosphorylated forms of Structural Maintenance of Chromosomes 1 (“Smc1”) (Smc1 pS957 and pS966), the generation of synthetic hybrid reference phosphopeptides, and the development of ELISA assays for use therewith.

Rationale:

As described in Example 1, it was determined that p-Smc1S957 and p-Smc1S966 are induced in cells exposed to radiation and in dogs exposed to total body irradiation. This Example describes the generation of reagents for measuring the presence and/or amount of Smc1 (pS957) and/or Smc1 (pS966) in a biological sample, including the use of synthetic reference phosphopeptide comprising the first epitope (capture) and either the second epitope (pS957) of interest or the third epitope of interest (pS966) from the Smc1 target protein, wherein the synthetic reference peptide is capable of simultaneously binding to both the capture reagent that binds to the first epitope and to at least one detection reagent that binds to the second or third epitope.

Methods:

1. Selection of Epitope #1 from Smc1 for Binding to a Capture Agent

The first epitope was derived from the carboxyl-terminus of the Smc1 protein, which is highly conserved between human, monkey, rabbit, dog, mouse and rat, as shown below in Table 1. “Reference ID” refers to the amino acid sequence of the full length Smc1 protein.

TABLE 1
Smc 1: Alignment of conserved Regions corresponding to Epitope 1
(Capture epitope)

| | | Region corresponding to FHC37F synthetic |
|---|---|---|
| organism | reference ID | peptide (capture epitope = epitope #1) |
| synthetic | FHC37F | DLTKYPDANPNPNEQ (SEQ ID NO: 1) |
| peptide | | |
| human | NP_006297.2 | DLTKYPDANPNPNEQ (SEQ ID NO: 1) |
| monkey | XP_001091228.1 | DLTKYPDANPNPNEQ (SEQ ID NO: 1) |
| rabbit | XP_002720089.1 | DLTKYPDANPNPNEQ (SEQ ID NO: 1) |
| dog | XP_538049.2 | DLTKYPDANPNPNEQ (SEQ ID NO: 1) |
| mouse | NP_062684.2 | DLTKYPDANPNPNEQ (SEQ ID NO: 1) |
| rat | NP_113871.1 | DLTKYPDANPNPNEQ (SEQ ID NO: 1) |

2. Selection of Epitope #2 for Detection of Phosphorylated Smc1 at Serine 957

An amino acid region corresponding to the second epitope of Smc1 was selected (SQEEGS[p]SQGEDSVSG) which corresponds to human Smc1 amino acids 951 to 965 and was synthesized with a phospho-serine at position 957.

TABLE 2
Smc 1: Alignment of conserved Regions corresponding to Epitope #2
(Detection of phosphorylated serine 957 (p957))

| | | Region corresponding to FHC37Cp synthetic |
|---|---|---|
| organism | reference ID | peptide (detection epitope = epitope #2) |
| synthetic | FHC37Cp | SQEEGSSQGEDS (SEQ ID NO: 14) |
| peptide | | |
| human | NP_006297.2 | SQEEGSSQGEDS (SEQ ID NO: 14) |
| monkey | XP_001091228.1 | SQEEGSSQGEDS (SEQ ID NO: 14) |
| rabbit | XP_002720089.1 | SQEEGSSQGEDS (SEQ ID NO: 14) |
| dog | XP_538049.2 | SQEEGSSQGEDS (SEQ ID NO: 14) |
| mouse | NP_062684.2 | SQEEGSSQGE [symbol] S (SEQ ID NO: 7) |
| rat | NP_113871.1 | SQEEG [symbol] SQGE [symbol] S (SEQ ID NO: 8) |

Note:
the amino acid sequence set forth as SEQ ID NO: 14 is fully contained within the amino acid sequence set forth as SEQ ID NO: 2.

3. Selection of Epitope #3 for Detection of Phosphorylated Smc1 at Serine 966

An amino acid region corresponding to the third epitope of Smc1 was selected (DSVSG[p]SQRISS) which corresponds to human Smc1 amino acids 961 to 971 with a phospho-serine at protein position 966.

TABLE 3
Smc 1: Alignment of conserved Regions corresponding to Epitope #3
(Detection of phosphorylated serine 966 (p966))

| | | Region corresponding to FHC37Dp synthetic |
|---|---|---|
| organism | reference ID | peptide (detection epitope = epitope #3) |
| synthetic | FHC37Dp | DSVSGSQRISS (SEQ ID NO: 3) |
| peptide | | |
| human | NP_006297.2 | DSVSGSQRISS (SEQ ID NO: 3) |
| monkey | XP_001091228.1 | DSVSGSQRISS (SEQ ID NO: 3) |
| rabbit | XP_002720089.1 | DSVSGSQR [symbol] SS (SEQ ID NO: 9) |
| dog | XP_538049.2 | DSVSGSQR [symbol] SS (SEQ ID NO: 9) |
| mouse | NP_062684.2 | [symbol] SVSGSQR [symbol] SS (SEQ ID NO: 10) |
| rat | NP_113871.1 | [symbol] SVSGSQR [symbol] SS (SEQ ID NO: 10) |

4. Generation of Anti-Phospho-Smc1 Antibodies that Bind to Epitope #2 (pS957) or Epitope #3 (p966) Using Synthetic Immunogenic Peptides

Monoclonal antibodies were generated against the serine phosphorylated forms of Smc1 as follows. Phosphorylated peptides encompassing the pS957 and pS966 amino acids of Smc1 and a peptide (not phosphorylated) corresponding to the capture epitope were synthesized as shown in Table 4 and the diagram illustrated in FIG. 2.

TABLE 4
Synthetic Immunogenic Peptides and Synthetic Reference
phospho-peptides

| | | SEQ |
|---|---|---|
| | | ID |
| Description | Sequence | NO: |
| Immunogenic peptide FHC37_F | DLTKYPDANPNPNEQ | 1 |
| (capture) (N term CGSG spacer | | |
| was added) | | |
| Immunogenic phospho-peptide | SQEEGS[p]SQGEDSVSG | 2 |
| FHC37_Cp/serine 957 | | |
| (detection) (N term CGSG spacer | | |
| was added) | | |
| Immunogenic phospho-peptide | DSVSG[p]SQRISS | 3 |
| FHC37_Dp/serine 966 | | |
| (detection) (N term CGSG spacer | | |
| was added) | | |
| synthetic peptide reference | ISQEEGS[p]SQGEDSDLTKYPDANPNPNEQ | 4 |
| standard FHC37_FCp | (phosphorylated serine 957) | |
| synthetic peptide reference | EDSVSG[p]SQRISSIDLTKYPDANPNPNEQ | 5 |
| standard FHC37_FDp | (phosphorylated serine 966) | |

Note:
[p]S: designates a phosphorylated serine residue

All peptides used for immunization, screening and standards were synthesized by Chinese Peptide Company (Hangzhou, China). Three peptides were used for immunization; the first peptide (SQEEGS[p]SQGEDSVSG) corresponds to human Smc1 amino acids 951 to 965, as included herein as SEQ ID NO:2, and was synthesized with a phospho-serine at position 957. The second immunization peptide (DSVSG[p]SQRISS) corresponds to human Smc1 amino acids 961 to 971, as included herein as SEQ ID NO:3, with a phospho-serine at protein position 966. The third immunization peptide (DLTKYPDANPNPNEQ) corresponds to the C-terminus of the Smc1 protein starting at position 1219, as included herein as SEQ ID NO:1. All three immunization peptides were synthesized with an N-terminal linked CGSG spacer, included herein as SEQ ID NO:13.

Two peptides were synthesized for counter screening and are the non-phosphorylated counterparts of the two phospho immunization peptide: (CGSGSQEEGSSQGEDS and CGSGDSVSGSQRISS, included herein as SEQ ID NOS:11 and 12, respectively). Two additional peptides were generated for reference standards (ISQEEGS-pS-QGEDSDLTKYPDANPNPNEQ, SEQ ID NOS:4, and EDSVSG-pSQ-RISSIDLTKYPDANPNPNEQ, SEQ ID NO:5). Both the reference standard peptides contain the C-terminal sequence of Smc1 (AAs 1219 to 1233) concatenated with the sequence surrounding pS957 or pS966. Standard peptide concentrations were determined by Amino Acid Analysis (New England Peptide, Gardner, Mass.).

The synthesized immunopeptides were conjugated through the N-terminal cystines to Keyhole Limpet Hemocyanin (KLH) and used to immunize 12, 3-4 month old female New England White rabbits at a commercial facility (Epitomics, Burlingame, Calif.). The rabbits were bled prior to immunization and then injected with the KLH-conjugated Smc1 peptides and boosted every 2-3 weeks for a total of 5-6 injections per rabbit. The rabbits were monitored for immune response by peptide ELISA, and were also counter-screened with the corresponding non-phosphorylated peptide. The rabbits were scored as passing the peptide ELISA screen based on empirical criteria (O.D. >0.30 for the 1:64,000 serum dilution).

Final sera from immunized rabbits were screened by Western Blot and Immunoprecipitation. Regarding the Western Blot (WB) analyses, whole cell lysates were isolated from human LBL at either 2 or 5 hours after treatment with mock or 10 Gy of IR (5.6 Gy/min). Protein lysates (25 to 50 mg/lane) were adjusted to 1× NuPAGE® LDS Sample Buffer containing NuPAGE® Sample Reducing Agent (Invitrogen, Carlsbad, Calif.) and heated to 98° C. for 5 minutes. Lysates were subjected to SDS-PAGE, transferred to nitrocellulose membranes using an XCell II™ Blot Module (Invitrogen). Membranes were placed in 50-ml conical tubes (Falcon 352070, Becton Dickinson, Franklin Lakes, N.J.) and blocked for 1 hour in SuperBlock (Pierce, Thermo Scientific, Rockford, Ill.) with 0.1% Tween 20 (Sigma, St. Louis, Mo.) on a rotisserie rotator (Barnstead/Thermolyne, Dubuque, Iowa) at room temperature. Blocking agent was aspirated away, and probed with Protein-A purified antibody isolated from pre- or post-immune rabbit sera. Protein-A purified antibody was diluted 1:500 and incubated overnight at 4° C. in 1×PBS, 10% SuperBlock and 0.1% Tween 20. Membranes were washed two times with 10 ml 1×PBS, 0.1% Tween 20. HRP-conjugated goat anti-rabbit secondary antibody (Cell Signaling Technology, Danvers, Mass.) diluted 1:2000 in 1×PBS, 10% SuperBlock and 0.1% Tween 20 was added to the membrane and incubated 1 hour at room temperature on a rotisserie rotator. Secondary antibody was aspirated away and the membrane was washed two times with 10 ml 1×PBS, 0.1% Tween 20, 5 min/wash. Then 1× LumiGLO substrate (Cell Signaling Technology, Beverly, Mass.) was added and incubated 5 minutes at room temperature on a rotisserie rotator. The membrane was then exposed to film (CLXPosure, Pierce), developed, scanned and digitized. Positive controls were run by blotting the same lysates with a commercial antibody that binds a phosphorylated form of Smc1. Commercial Smc1 antibodies were purchased from Cell Signaling Technologies (Danvers, Mass.) and used at the manufacturer's recommended dilutions. Additionally, the specificity of anti-phospho antibodies were confirmed by including a control lysate from cells treated with 10 Gy of IR followed by treatment with λ-phosphatase. An immunized rabbit was scored as positive by WB screen if the post-immune sera gave a signal at the appropriate molecular weight and the signal was absent from the corresponding pre-immune Western Blot.

Regarding Immunoprecipitation (IP) analyses, antibody was first purified from rabbit sera and hybridoma supernatants using HiTrap Protein A HP columns (GE Healthcare, Piscataway, N.J.). Briefly, sera were diluted 1:2 with phosphate-buffered saline (PBS) prior to loading. Hybridoma supernatants (2 to 45 ml) were loaded directly onto prewashed columns. The column was washed with PBS and eluted with 0.1 M citric acid (pH 3.0). Three 0.5-ml fractions were collected in tubes containing 0.125 ml 1 M Tris-HCl (pH 9.0) to give a final pH of 7.4. Protein concentration was determined with the Bradford assay (BioRad, Hercules, Calif.), and the two or three most concentrated antibody fractions were pooled. Antibody concentration was determined by the Bradford assay (BioRad) or by OD280 using a bovine gamma globulin IgG (Pierce, Thermo Scientific, Rockford, Ill.) standard curve. Detection antibodies specific for FHC37_Cp and FHC37_Dp were biotinylated with the FluoReporterH Mini-Biotin-XX Protein Labeling Kit (Invitrogen, Carlsbad, Calif.).

Pre- or post-immune antibodies purified from sera by Protein-A affinity columns were used to immunoprecipitate Smc1 from protein lysates. The immunoprecipitate complex was brought down with either Protein-A or Protein-G agarose beads. Specifically, 30 microliters of protein-A beads (Invitrogen) were washed 2× in PBS and then incubated with 50 mg of protein lysate in a volume of 100 ml for 1 hour at 4° C. with mixing by end-over-end tumbling. The protein-A beads were pelleted by centrifugation and the lysates were transferred to a fresh tube and incubated with 30 ml of serum or hybridoma supernatant for 1 hour at 4° C. with mixing by tumbling. An additional 30 ml of protein-A beads were washed 2× in PBS and then added to the lysate/antibody mix and incubated for an additional hour at 4° C. with mixing by tumbling. The protein-A beads were pelleted by centrifugation and washed 2× in PBS, and the antigen was recovered by heating to 98° C. for 5 min in 1×LDS loading buffer. Proteins were transferred to nitrocellulose membranes, blocked, and then probed with an antibody directed toward the target protein of interest. A rabbit was scored as positive by immunoprecipitation if the post-immune sera gave a signal at the appropriate molecular weight and the signal was absent from the corresponding pre-immune WB. Additionally, phospho-specificity of the antibodies was established if they resulted in an enriched signal in the 10 Gy lysate relative to the mock irradiated and the λ-phosphatase treated lysate.

5. Peptide Competition Assay

Protein lysates were generated from LBL GM10834 at 4 hours after exposure to 5 Gy. Lysates were diluted 1:80 in dilution buffer and either competitive peptide or the nonspecific control peptide was added at multiple concentrations ranging from 2 pM up to 20 nM. The amount of endogenous phosphorylated Smc1 protein (phospho-Smc1 pS957 and phospho-Smc1 pS966) was measured by ELISA.

6. Mixing Experiment

Protein lysates were generated from LBL GM10834 4 hours after mock exposure or exposure to 5 Gy. The lysates were diluted either 1:160 (phospho-Smc1 (pS957) assay) or 1:40 (phospho-Smc1 (pS966) assay) in dilution buffer and then mixed at different ratios. The concentration of phosphorylated Smc1 protein was determined by ELISA and quantified by the standard peptide curve.

7. Standard Addition Experiment

Standard peptide was added to either cell lysate from mock irradiated LBL GM10834 (diluted 1:20 in dilution buffer) or directly to dilution buffer. The concentration of the spiked-in standard peptides was determined by ELISA and quantified using the standard peptide curve.

Results:

Of the 12 rabbits immunized with KLH-conjugated Smc1 peptides, 11 passed the Western blot and immunoprecipitation quality control analysis. Based on the screening data, three rabbits were selected for monoclonal antibody (mAb) production. Specifically, one (1) rabbit was selected for the generation of the pan (capture) mAb (i.e., capture agent that specifically binds to epitope #1), and the remaining two rabbits were selected for the generation of the two different phospho-specific mAbs (i.e., detection agents that bind to epitope #2 or epitope #3).

Primary hybridoma lines from the selected immunized rabbits were created and screened. Briefly, lymphocytes were isolated from the spleens of selected animals and fused to create rabbit hybridoma lines grown in multi-well plates that contained 1-5 clones per well. The goal of this step in the process was to identify the 3 best candidate hybridoma lines (and up to 3 backup lines) to be sub-cloned to generate monoclonal hybridoma lines. The supernatants from these multi-clone hybridoma lines were screened by a combination of peptide ELISA, IP, and Western Blot. A subset of the primary hybridoma lines were subcloned by serial dilution. Supernatants from sub-clones were screened by peptide ELISA, IP, and Western Blot to identify hybridoma lines generating the correct mAb.

ELISA assays were developed using the mAbs specific for Smc1, including pan (capture) mAb (i.e., capture agent that specifically binds to epitope #1), and the two different phosphoserine-specific mAbs (i.e., detection agents that bind to epitope #2 or epitope #3). Specifically, ELISAs were developed in 96-well Costar (EIA/RA Plate no. 3369) plates using rabbit monoclonal antibody FHC37F-6-3 as a capture antibody and biotinylated rabbit monoclonals FHC37 Cp-33-1 and FHC37Dp-202-3 as the detection antibodies. See Table 5. Hybrid phosphopeptides were used as calibration standards. Multiple parameters were optimized in an iterative process in which two parameters at a time were compared before moving on to the next set of parameters. This process was repeated multiple times until the overall assay was optimized. For example, the initial parameter optimized was the concentration of capture Ab. Using high concentrations of sample (cell lysate or synthetic peptide), the amount of capture Ab per well was varied. A plot of the antibody concentration versus the signal for each sample concentration revealed that mAb concentration becomes limiting between 200 and 300 ng/well (not shown). Subsequently, the optimal dilution of biotinylated detection Ab was determined to be 1:4,000 by plotting the signal to noise ratio we detect an optimal detection Ab concentration for each batch of labeled detection Ab (not shown). Other parameters optimized along these lines include: ELISA plate composition, capture Ab binding buffer, blocking buffer composition, cell lysate buffer, sample concentration, sample incubation time, sample incubation temperature, standard curve dynamic range, detection Ab labeling method, HRP enzyme conjugate, different TMB substrate sources, and substrate development times. The key parameters affecting assay sensitivity were capture Ab concentration, detection Ab concentration, and sample concentration. Overall, 2 ELISAs were constructed and optimized for quantifying phospho-Smc1: p-Smc1 (pS957) and p-Smc1 (pS966).

TABLE 5
ELISA Assays for detecting pS957 or pS966

| | | | | | | | | | Protein |
|---|---|---|---|---|---|---|---|---|---|
| | | | | | | | | | level of |
| | Capture Ab | | Detection | | HRP | | Peptide | | detection |
| Assay | | ng/ | Ab | | Conjugate | | | linear range | (×10⁶ |
| Target | clone | well | clone | dilution | conj. | dilution | name | (fmol/well) | cells |
| Smc1 | FHC37F-6-3 | 250 | FHC37Cp-33-1 | 1:2,000 | avidin | 1:4000 | Smc1_Cp_F | 2 to 0.03 | 0.05 |
| (pS957) | | | | | | | | | |
| Smc1 | FHC37F-6-3 | 250 | FHC37Dp-202-3 | 1:2,000 | avidin | 1:4000 | Smc1_Dp_F | 2 to 0.03 | 0.05 |
| (pS966) | | | | | | | | | |
| Smc1 | FHC37F-6-3 | 250 | commercial 41J | 1:500 | -mouse | 1:4000 | TBD | TBD | TBD |
| (pan) | | | | | IgG | | | | |

A detailed ELISA protocol is as follows:

1. Coat polystyrene 96-well plates (Corning, Corning, N.Y.) overnight at 4° C. with 50 ml/well with FHC37F-6-3 antibody diluted to 6 mg/ml in PBS.

2. Wash plate three times with 300 ml/well with 1×PBS, 0.05% Tween 20 using an automated plate washer (BioTek ELx405™, Winooski, Vt.).

3. Block plates for 1 hour at room temperature on an orbital shaker with 150 ml/well Blocking Buffer [10% SuperBlock (Pierce), 0.1% Tween 20 (Sigma)].

4. Wash plates three times with automated plate washer.

5. Prepare standard phospho-peptide curve by twofold serial dilution in Diluent Buffer (1 mM EDTA, 0.005% Tween 20, 0.5% Triton X-100, 1×PBS, 1% BSA).

6. Dilute protein lysates in Diluent Buffer and add 50 ml/well; incubate 1 hour at room temperature on an orbital shaker.

7. Wash plates three times with automated plate washer.

8. Dilute biotinylated detection antibodies FHC37 Cp-33-1 (242 ng/ml) or FHC37Dp-202-3 (182 ng/ml) in Diluent Buffer, add 50 ml/well and incubate 1 hour at room temperature on an orbital shaker.

9. Wash plates three times with automated plate washer.

10. Dilute streptavidin-conjugated HRP (Invitrogen) 1:2000 in Diluent Buffer and incubate 1 hour at room temperature on an orbital shaker.

11. Wash plates three times with automated plate washer.

12. Add 50 ml/well TMB substrate (Sigma) and incubate at room temperature for 1 to 5 minutes. Reaction is stopped by the addition of 50 ml/well of 0.4 N HCl.

13. Measure OD 450 on for end point assays or OD 640 every 40 seconds over 12 minutes for kinetic assays on a BioTek Synergy2 plate reader.

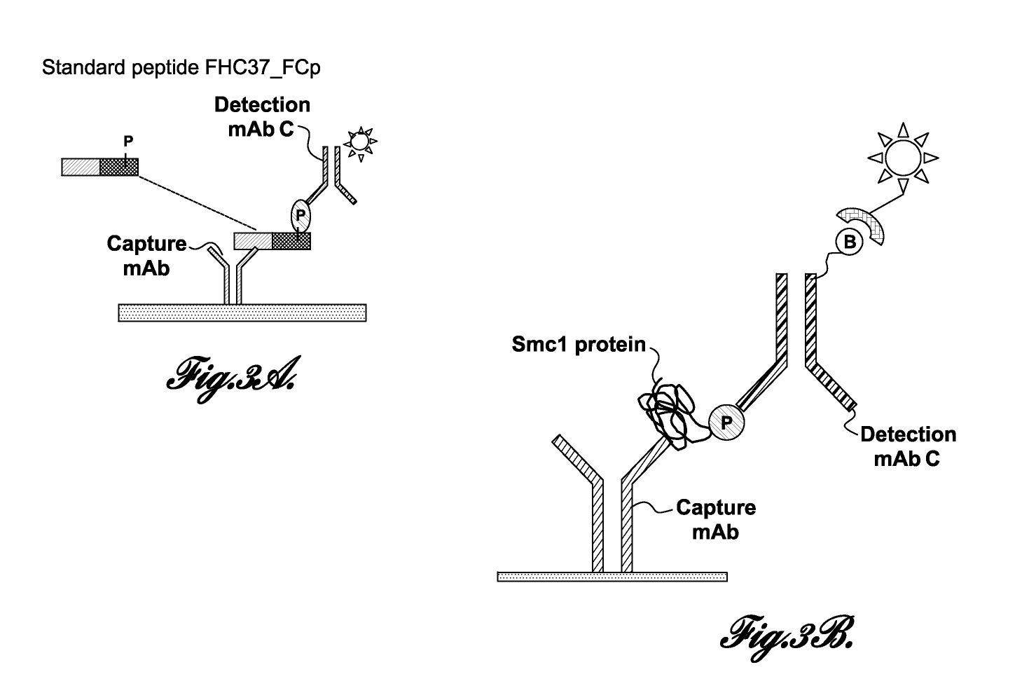

Initially, production of recombinant phospho-Smc1 was pursued for use as a standard for the ELISAs. The Smc1 gene was inserted into an expression vector, and the sequence was verified. However, due to the known difficulties and expense associated with the expression and purification of recombinant proteins, synthetic phosphopeptide standards were generated as shown above in Table 4 and their efficacy was verified. As shown in Table 4 and FIGS. 2-3, synthetic reference phosphopeptides were generated, each reference phosphopeptide comprising two independent epitopes corresponding to the sequence recognized by the ELISA capture antibody (the first epitope) and the sequence recognized by the ELISA detection antibody (the second or third epitope). For ELISAs employing the pS957 mAb (detection agent), the synthetic reference phosphopeptide (SEQ ID NO:4) included a first N-terminus comprising the second epitope (SEQ ID NO:2) from Smc1 (pS957) and a second C-terminus comprising the first epitope (SEQ ID NO:1). The serine residue corresponding to S 957 of the full length Smc1 polypeptide was synthesized using a phospho-serine amino acid.

Similarly, for ELISAs employing the pS966 mAb (detection agent), the synthetic reference phosphopeptide (SEQ ID NO:5) included a first N-terminus comprising the third epitope (SEQ ID NO:3) from Smc1 (pS966) and a second C-terminus comprising the first epitope (SEQ ID NO:1). The serine residue corresponding to S 966 of the full length Smc1 polypeptide was synthesized using a phospho-serine amino acid.

As shown in Table 4 and FIG. 2, the first and second epitopes in the synthetic reference phosphopeptide (for binding to the capture and detection agents, respectively) are separated by a spacer region of 1 to 50 amino acids to reduce the possibility of steric hindrance between the antibodies.

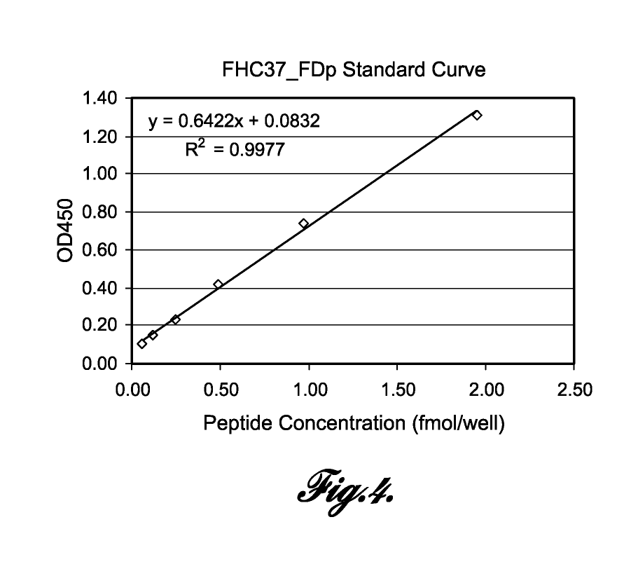

The concentrations of the hybrid reference peptide standards can initially be determined by amino acid analysis. The reference standard peptides are then added to the ELISA plates and serially diluted two-fold to generate a seven point standard curve. The sample concentration is calculated by selecting the linear range of the standard curve (4 to 6 points) and deriving the equation of that line. See FIG. 4. The mAbFHC37-F was used as the capture reagent and mAb FHC37_Dp-biotin was used as a detection reagent specific for phosphorylated serine 966. The standard curve was generated by plotting the concentration of the synthetic reference phosphopeptide (SEQ ID NO:5) versus OD450, a measure of the formation of a binding complex between the capture mAb, the synthetic reference phosphopeptide (SEQ ID NO:5), and the biotinylated detection mAb (e.g., as illustrated in FIG. 3B).

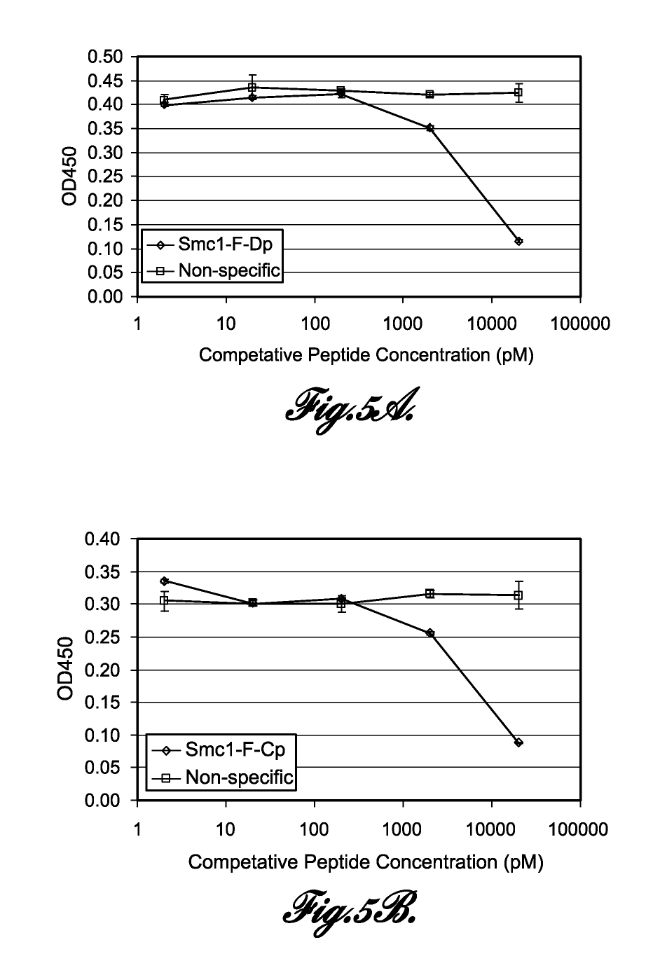

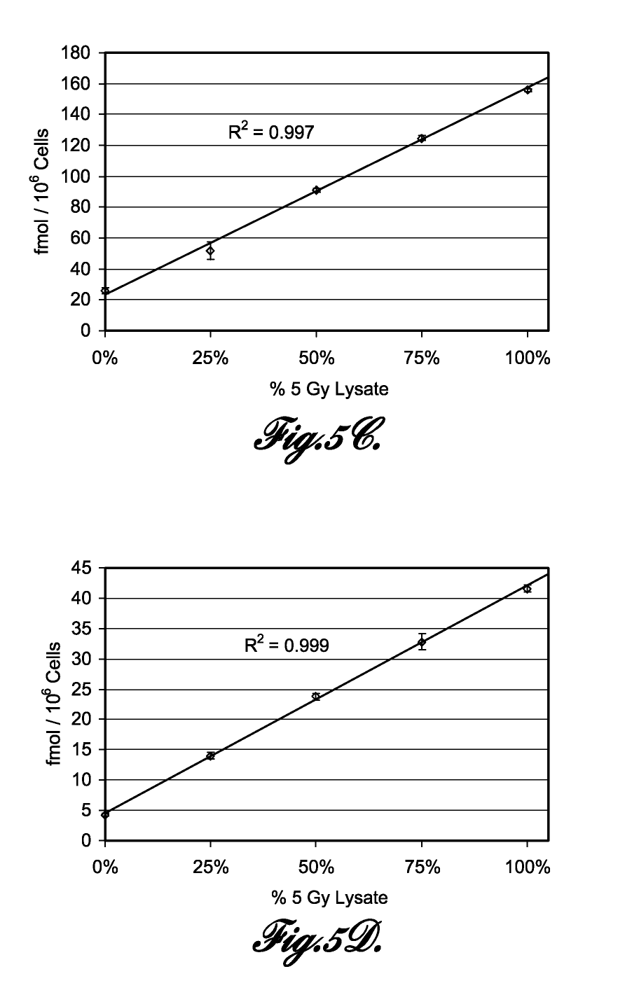

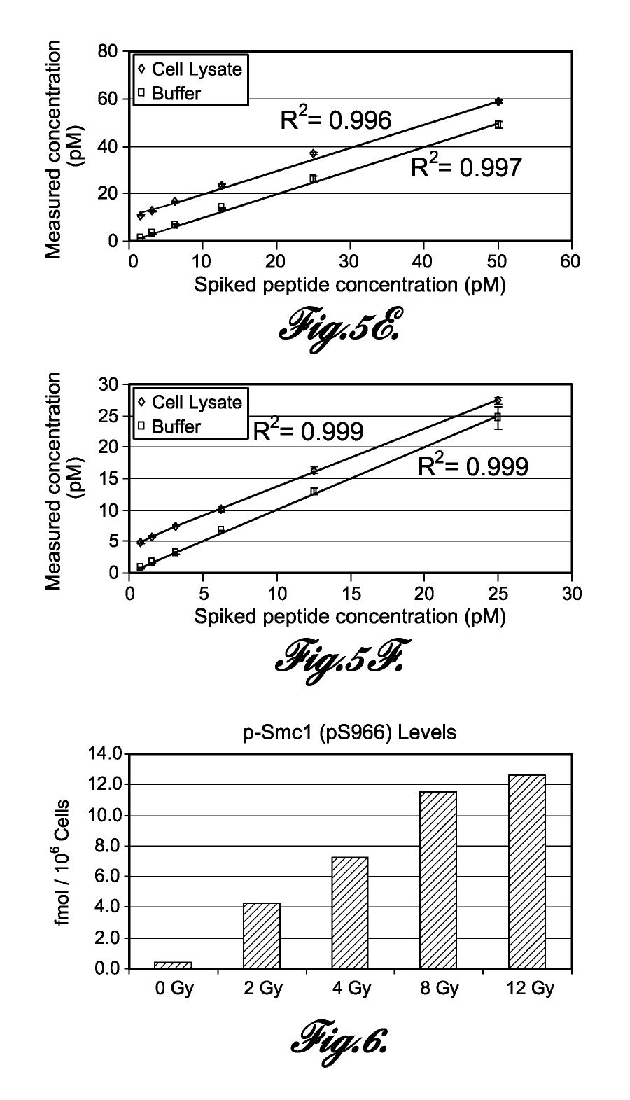

Specificity of the ELISAs was established by competition experiments. Protein lysate derived from LBL GM10834 cells 4 hours after exposure to 5 Gy was spiked with increasing concentrations of either a competitive peptide or a nonspecific control peptide. Lysates were diluted 1:80 in dilution buffer containing the indicated concentration of competitive or nonspecific peptide, and concentrations of endogenous phosphorylated Smc1 protein were measured by ELISA. The competitive peptide for the phospho-Smc1 (pS957) was the standard peptide used in the phospho-Smc1 (pS966) assay, Smc1_F_Dp. This peptide contains the epitope recognized by the capture antibody coupled with the phospho-epitope recognized by the detection antibody FHC37Dp-202-3. With increasing concentrations of the Smc1_F_Dp peptide, there was a decrease in signal detected for the endogenous phosphorylated Smc1 (pS957) protein as measured by ELISA (FIG. 5A). Similarly, when the lysate was spiked with increasing concentrations of the Smc1_F_Cp peptide, there was a similar competitive loss in the ability to measure levels of the endogenous phosphorylated Smc1 (pS966) protein (FIG. 5B). Moreover, the specificity of the ELISA for Smc1 was confirmed in four additional ways (data not shown). First, it was demonstrated that if the wells of the ELISA plate were blocked with BSA and not coated with capture antibody before the addition of sample (i.e., protein lysate from irradiated LBL or standard peptide), no signal above background was detected. Second, it was demonstrated that if protein lysates or peptides were incubated with a molar excess of capture antibody before being added to wells coated with the capture antibody, no signal above background was detected. Both results confirmed that the FHC37F-6-3 capture antibody is required for detection. Third, it was demonstrated that when the sample (either lysate from irradiated LBL or standard peptide) was incubated with a molar excess of unlabeled detection antibody before the addition of the biotinylated detection antibody, no signal above background was detected. Finally, the phospho-specificity of the ELISA assays was evaluated by treating protein lysates or standard phospho-peptides with λ-protein phosphatase. When dephosphorylated protein lysates or peptides were used, no signal above background was detected.

The linearity of the assays was demonstrated using standard mixing experiments. Protein lysates derived from LBL GM10834 cells 4 hours after exposure to 5 Gy or mock-irradiated were mixed at different ratios, and the levels of phosphorylated Smc1 protein were determined by the phospho-Smc1 (pS957) ELISA (FIG. 5C) or the phospho-Smc1 (pS966) ELISA (FIG. 5D) using external peptide calibration curves. The R-squared values were greater than 0.99 for both assays.

The recovery of the assays was demonstrated using standard addition of the control peptide to the lysate matrix. Standard peptide was added either to protein lysate from mock-irradiated LBL GM10834 or to dilution buffer (the standard curve). The concentrations of spiked-in Smc1_F_Cp peptide in each sample were measured using the phospho-Smc1 (pS957) assay (FIG. 5E), and spiked-in phospho-peptide Smc1_F_Dp concentrations were measured by the phospho-Smc1 (pS966) assay (FIG. 5F). The results represent triplicate measurements, and error bars represent standard deviations. The offset of the cell lysate relative to the buffer is due to low levels of endogenous phospho-Smc1 protein in the cell lysate.

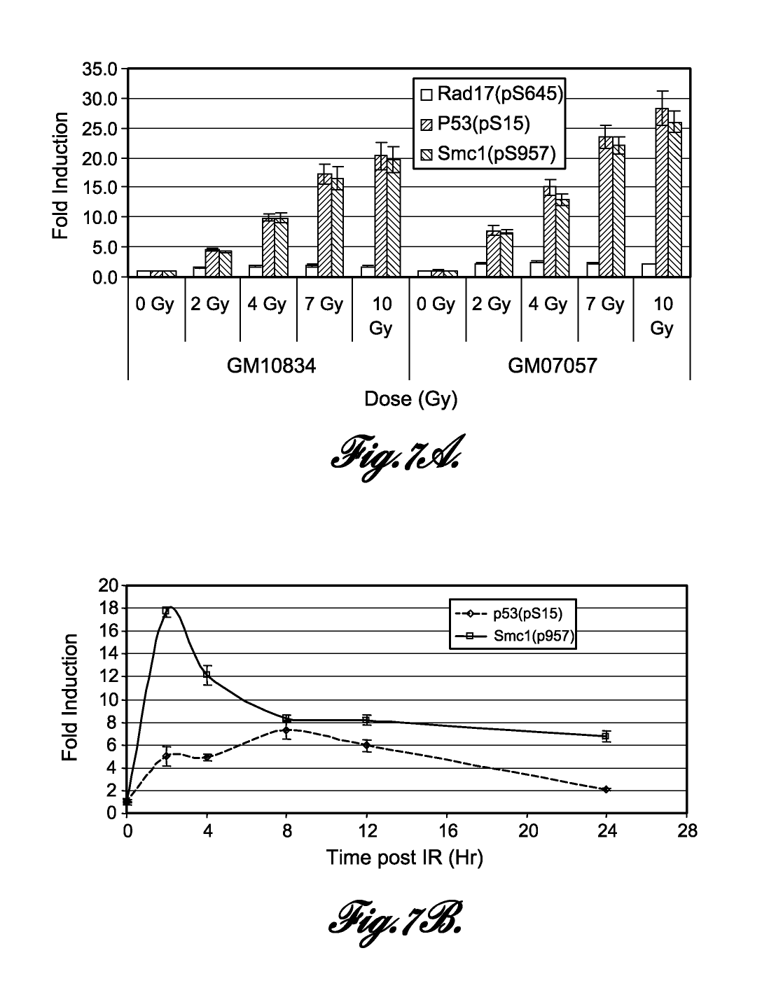

FIG. 6 graphically illustrates phospho-Smc1 (pS966) concentration in Lymphoblast Cell Line (LBL) derived from the standard curve. An actively growing LBL was divided into five separate treatment flasks and either mock irradiated (0 Gy) or treated with the indicated dose of ionizing radiation (IR). Cells were harvested four hours after irradiation and protein lysates were prepared. p-Smc1 (pS966) levels were determined by ELISA: The OD450 value for each lysate was converted to a molar concentration by way of the equation of the line generated with the standard reference peptide illustrated in FIG. 4.

As described above, the synthetic reference phosphopeptides were useful for generating a standard curve (see FIG. 4), which allows for the normalization of the amount of analyte protein within and between ELISA plates.

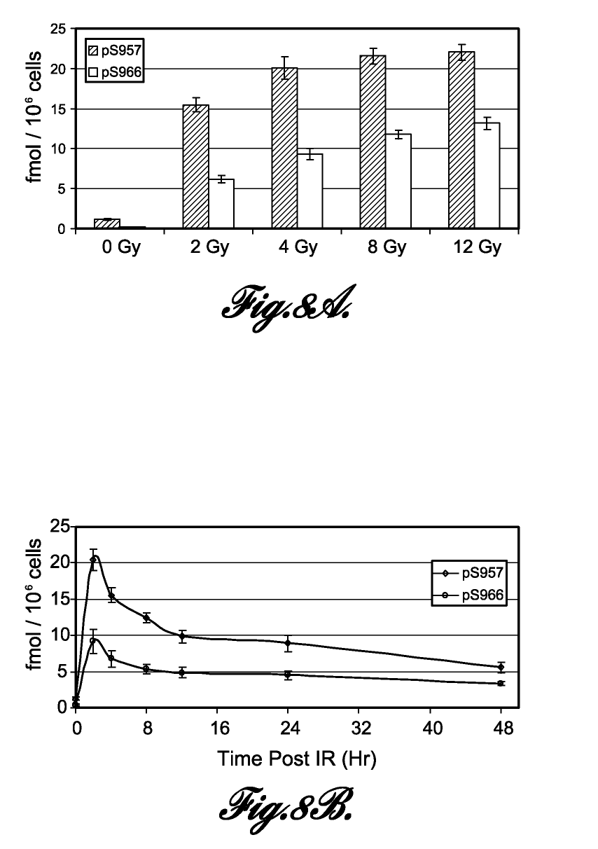

ELISAs were first validated using multiple human LBL cells post-IR, capturing the IR dose- and time-dependence of the phospho-Scm1 signals, as shown in FIG. 7. Regarding the IR dose-dependent signal, the fold induction of phospho-Smc1 (and of two other phosphoproteins, -p53 and -Rad17) was measured with the ELISA in two independent LBLs (GM10834, GM07057). LBLs were exposed to mock IR, or IR at 2, 4, 7 or 10 Gy. Two sets of lysates were harvested for each LBL at 4 hrs post-IR, and were analyzed in triplicate using the ELISAs described above (FIG. 7A). The ELISA clearly illustrates the IR dose dependence of phospho-Smc1 induction. Regarding time-dependent signal, fold induction of phospho-Smc1 (and phospho-p53), LBL cells were mock-irradiated or treated with 5 Gy of IR. Cells were harvested at 2, 4, 8, 12, and 24 hours post-IR. Phospho-protein levels were measured with the ELISA assays in duplicate on two independent plates. Fmol of phospho-protein per ng of lysate were calculated from the standard curve and fold induction was calculated by normalizing to the mean value for the mock-irradiated samples (FIG. 7B). The results from the ELISA assay clearly demonstrates the time dependence of phospho-Smc1 induction after IR dose. Specifically, phospho-Smc1 is induced up to 18-fold at two hours post-IR at 5 Gy. By 8 hours post-IR, the phospho-Scm1 induction level drops to 8-fold over pre-IR, and induction levels gradually reduce to about 7-fold after 24 hours post-IR. For both A and B, mean fold induction levels are plotted, and error bars are 1 standard deviation of the mean.

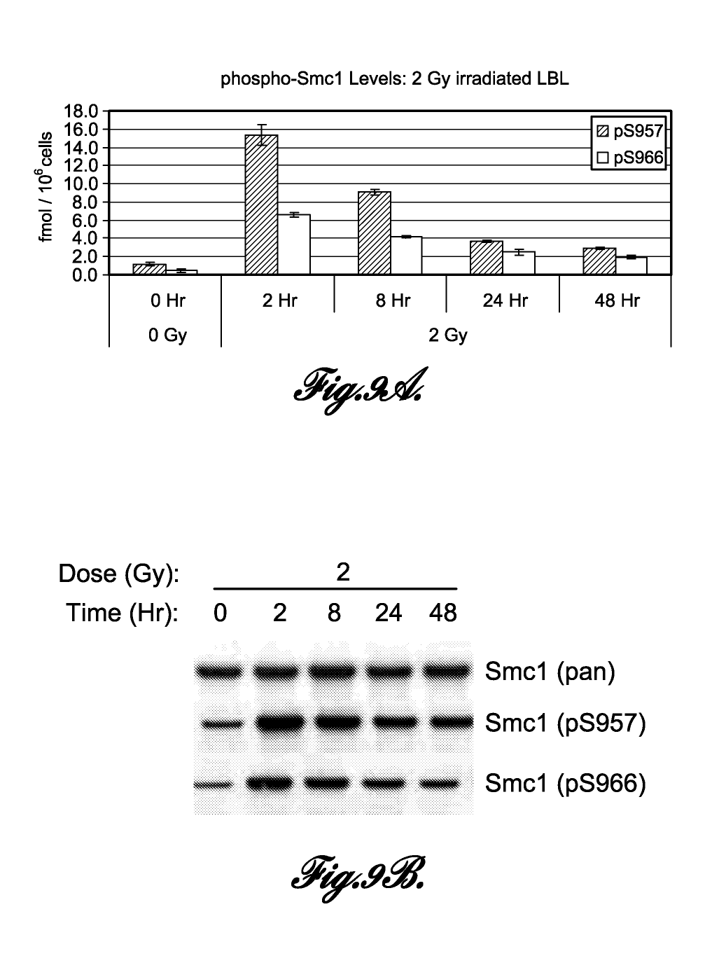

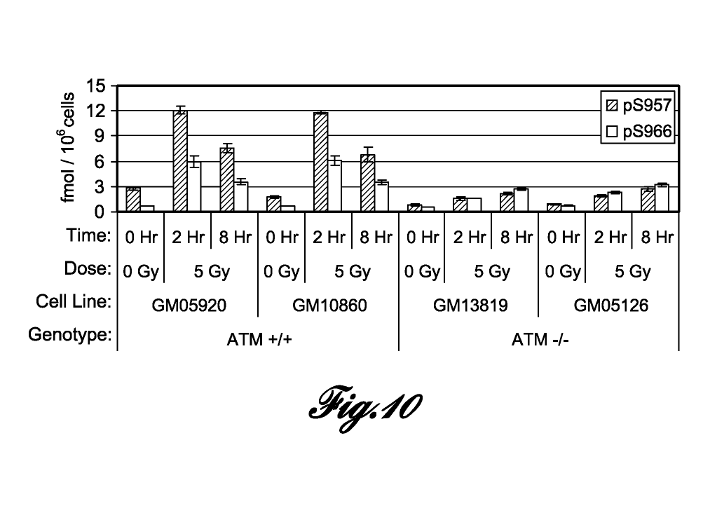

FIGS. 8-9 illustrate an expanded data set that further validates the ability of the ELISAs to detect IR dose- and time-dependence of the phospho-Smc1 signals. Two sets of protein lysates were generated from GM07057 cells 2 hours after mock irradiation (0 Gy) or irradiation (2-12 Gy). Lysates were evaluated by ELISA for Smc1 phosphorylation at pS957 and pS966. Each lysate was run in triplicate on two independent plates. The mean concentrations of phospho-Smc1 (pS957) and phospho-Smc1 (pS966) were calculated from the standard peptide curve, and the values were normalized to cell count. Values are means±SD. The average inter-well variation of the measurement was 2.2% for both assays for all dilutions of all lysates across all four plates. The average inter-plate concentration variation was 6% for the phospho-Smc1 (pS957) assay and 4% for the phospho-Smc1 (pS966) assay for all lysates across all plates. As illustrated in FIG. 8A, both phospho-Smc1 (pS957) and phospho-Smc1 (pS966) ELISAs detected a dose-dependent accumulation of their respective phospho-analytes from IR doses of 0, 2, 4, 8, and 12 Gy. The radiation-induced level of phospho-Smc1 (pS957) was approximately two-fold higher than that of phospho-Smc1 (pS966) across the dose range tested. The baseline level of phospho-Smc1 (pS957) (i.e., in the mock-irradiated sample) was higher than that of phospho-Smc1 (pS966). Hence, at 12 Gy there was a 50-fold increase in phospho-Smc1 (pS966) analyte compared to the mock-irradiated sample, while there was a 19-fold increase for the phospho-Smc1 (pS957).

Additionally, a time course study after 5 Gy irradiation revealed parallel kinetic responses for both phospho-Smc1 (pS957) and phospho-Smc1 (pS966). Two sets of protein lysates were generated from GM07057 cells at 2, 4, 8, 12, 24, and 48 hours after mock irradiation (0 hour samples) or irradiation with 5 Gy. Lysates were evaluated by ELISA for Smc1 phosphorylation at pS957 and pS966. Each lysate was run in triplicate on two independent plates. The mean concentrations of phospho-Smc1 (pS957) and phospho-Smc1 (pS966) were calculated from the standard peptide curve, and the values were normalized to cell count. The average inter-well variation of the measurement was 2.6% for the pS957 assay and 8.6% for the pS966 assay for all dilutions of all lysates across all four plates. The average inter-plate variation was 8.6% for the phospho-Smc1 (pS957) assay and 11.6% for the phospho-Smc1 (pS966) assay for all lysates across all plates. As illustrated in FIG. 8B, the levels of both analytes peaked by 2 hours after radiation exposure, with the phospho-Smc1 (pS957) ELISA showing a 16-fold induction over baseline and the phospho-Smc1 (pS966) showing a 24-fold induction over baseline. Although the response gradually trailed off after peaking by 2 hours post-irradiation, both phospho-Smc1 (pS957) and phospho-Smc1 (pS966) remained five-fold elevated 48 hour after exposure.

The above ELISA results illustrated in FIG. 8 were corroborated by additional ELISAs and parallel Western blotting performed on an independent set of lysates. See FIGS. 9A and 9B. Protein lysates were generated from GM07057 cells at 2, 8, 24, and 48 hours after mock irradiation (0 h) or exposure to 2 Gy IR. Lysates were evaluated for Smc1 phosphorylation at pS957 and pS966 by either ELISA or Western blotting. For ELISA, each lysate was run in triplicate. The mean concentrations of phospho-Smc1 (pS957) and phospho-Smc1 (pS966) were calculated from the standard peptide curve, and the values were normalized to cell count. The error bars are ±SD. See FIG. 9A. For Western blot analysis, 10 mg of each lysate was resolved by SDS-PAGE. The anti-Smc1 capture antibody (FHC37F) was used to evaluate total Smc1 levels, while the two detection antibodies (FHC37 Cp and FHC37Dp) were used to evaluate the levels of phospho-Smc1 (pS957) and phospho-Smc1 (pS966). See FIG. 9B. As above in FIG. 8, both ELISA and Western blots illustrate that the levels of phospho-Smc1 (pS957) and phospho-Smc1 (pS966) peak at 2 hours post exposure and gradually decline thereafter, but still maintain elevated levels by 48 hours post exposure.

It is noted that cells from patients afflicted with the genetic disorder ataxia telangiectasia (AT) are severely defective in Smc1 phosphorylation in response to ionizing radiation due to a lack of ATM-encoded kinase activity. To determine whether the phospho-Smc1 (pS957) and phospho-Smc1 (pS966) ELISAs could detect this deficiency in AT patient-derived cells, these phosphoanalytes were measured in protein lysates derived from ATM+ and ATM− lymphoblasts at 2 and 8 hours after exposure to 5 Gy radiation. Specifically, two independent sets of protein lysates were generated from cells of each of four lymphoblast cell lines (ATM+: G05920 and GM10860; ATM−: GM13819 and GM05126) at 2 and 8 hours after exposure to 5 Gy. Control cells were mock-irradiated. Lysates were evaluated by ELISA for Smc1 phosphorylation at pS957 and pS966. Each lysate was run in duplicate and the concentrations of phospho-Smc1 (pS957) and phospho-Smc1 (pS966) were calculated from the standard peptide curve. Phospho-Smc1 levels across both lysates are plotted as means±SD. As expected, the ATM− lymphoblasts showed very low levels of induction of phospho-Smc1 (pS957) and phospho-Smc1 (pS966) compared with ATM+ cells (FIG. 10). The low level of phospho-induction in ATM− cells has been described and is likely due to redundant kinase activity of other PI3-kinase family members.

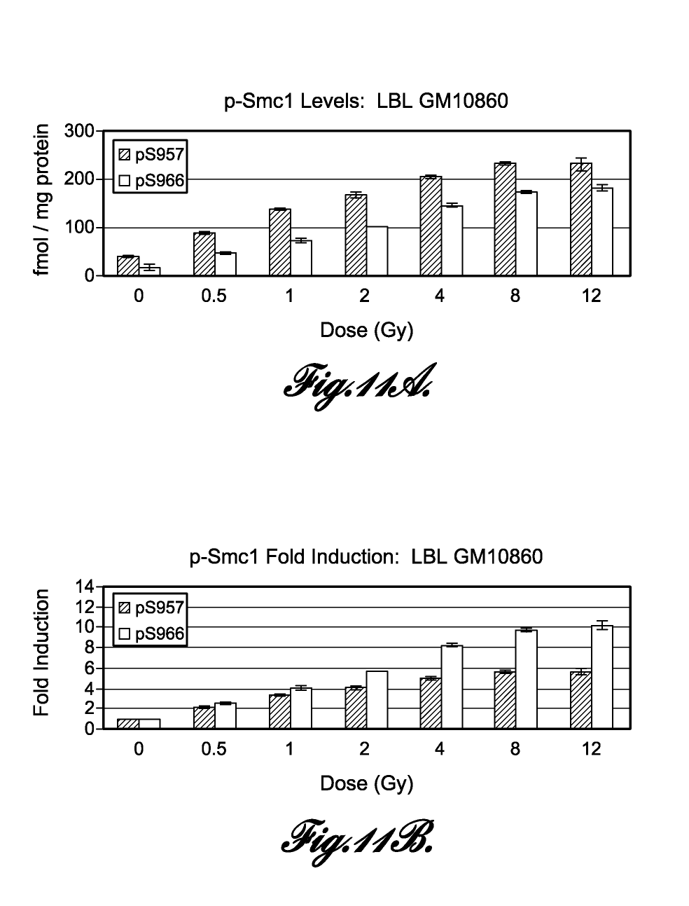

As illustrated in FIG. 11, the ELISAs are able to detect elevated levels of phospho-Smc1 (pS957) and phospho-Smc1 (pS966) in human lymphoblast cells after exposure to IR as low as 0.5 Gy. Briefly, protein lysates were generated from LBL GM010860 cells at one hour after mock irradiation (0 hour) or exposure to 0.5, 1, 2, 4, 8 and 12 Gy IR. The lysates were evaluated by ELISA for Smc1 phosphorylation at pS957 and pS966. The elevated levels of phospho-Smc1 (pS957) and phospho-Smc1 (pS966) are illustrated as a function of mg lysate protein (FIG. 11A). Fold induction was calculated over baseline (0 Gy) (FIG. 11B). With exposure to 0.5 Gy IR, the ELISAs detected approximately 2, and 2.5-fold induction of phospho-Smc1 (pS957) and phospho-Smc1 (pS966), respectively.

Conclusion:

In summary, monoclonal antibodies to two phosphorylated forms of Smc1 (pS957 and pS966) were successfully generated. ELISAs were developed and optimized using the monoclonal antibodies and novel phospho-polypeptide standards. Specificity of the ELISAs was demonstrated with competition assays. Furthermore, the ELISAs were validated for the ability to detect the time- and dose-dependent phosphorylation of Smc-1 in multiple human LBL cultures due to IR exposure. It is noteworthy that these ELISAs were able to detect phosphorylation of both Smc1 targets with as little as 0.5 Gy IR, and could detect elevated levels for as long as 48 hours post exposure. These data demonstrate the sensitivity and efficacy of the ELISAs to detect Smc1 phosphorylation in cells in response to a physiologically relevant range of IR doses.

### Example 3

This Example describes the use of murine and canine models to demonstrate efficacy of the Smc1 ELISA assay to detect induction of phospho-Smc1 after ionizing radiation exposure in vitro, ex vivo, and in vivo.

Methods:

Murine in vivo studies were used to validate the efficacy of the Smc1 ELISAs, and to detect the induction of phospho-Smc1 in mammalian cells after total body IR exposure. Ten Gy of total body irradiation (TBI) was applied to a C57 b16 mouse. Additionally, 2.75 Gy TBI was applied to one mouse of each strain NSG1 and NSG2, for a total of two mice. Blood samples were obtained pre-TBI exposure and approximately 1 hour post-TBI exposure. Lysates were prepared from the blood samples, and the levels of phospho-Smc1 (pS957) and phospho-Smc1 (pS966) were determined using the ELISAs described above in Example 2. Levels of phospho-Smc1 were normalized to pre-TBI levels.

A canine model was also used to validate the efficacy of the Smc1 ELISA assay, (described in Example 2) to detect induction of phospho-Smc1 (pS957) in mammalian cells after IR exposure. For the canine in vivo studies study, TBI was applied at 2, 6, or 10 Gy, delivered at a dose rate of either 7 cGy/minute or 70 cGy/minute. Blood samples were obtained pre-TBI exposure and at 0.5, 2.5, 4.5, 6.5, 10.5, 24, 48, and 68 hours post-TBI

For the ex vivo studies, whole blood was obtained pre-TBI, divided into aliquots, and exposed ex vivo to 2, 6, or 10 Gy IR at a dose rate of either 8.5 cGy/minute or 66 cGy/minute, and protein lysates were prepared at 0.5, 2.5, 4.5, 6.5 hours post-IR. For the in vitro studies, blood was obtained pre-TBI. PBMC were isolated on Ficoll gradients, activated in vitro with anti-canine CD3/CD28 antibodies, and cultured. After 10 days, the cultured PBMCs were exposed to 2, 6, or 10 Gy, delivered at rates of 8.5, 66, or 529 cGy/minute. Protein lysates were prepared immediately after the completion of IR and at 0.5, 2.5, 4.5, 6.5 hours post-IR. Phospho-Smc1 (pS957) levels were determined for all assays in cell lysates using the quantitative ELISA assay described above.

The rationale for choosing the radiation type (γ rays), exposure type (pulse or continuous), and dose rates was as follows: (1) The radiation dose from prompt neutrons dissipates rapidly as one moves from the hypocenter 40, such that the vast majority (98%) of the total dose comes from γ rays at distances where potential survivors will be located. In addition, radiological terrorism from a “dirty” bomb or a contaminated food supply is probably more likely than a nuclear bomb, and many of the currently available sources of radiation material for “dirty” bombs emit γ rays. A single burst of exposure was chosen for the initial experiments; in the event of a nuclear explosion, most of the total dose from γ rays (both prompt primary and secondary) occurs over a relatively short time; in Hiroshima and Nagasaki, there was little contribution to the dose beyond 40 s⁴⁰. Although the dose rates used in this Example do not match the delivery rate from a nuclear explosion, different types of radiological terrorism (i.e., dirty bomb, contaminated food supply) may be at a similar or slower rate of exposure employed herein.

Results:

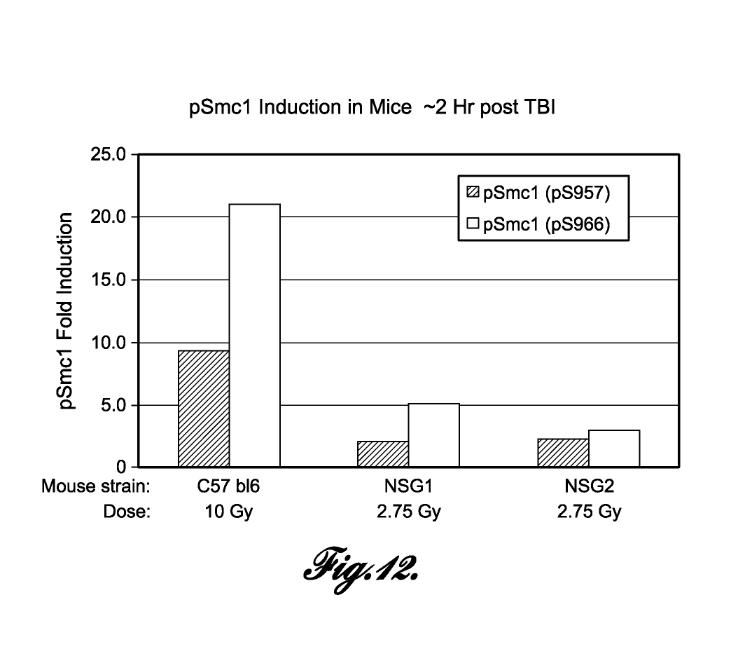

As illustrated in FIG. 12, ELISAs detected increased levels phospho-Smc1 (pS957) and phospho-Smc1 (pS966) in mice exposed to total body irradiation. The ELISAs detected approximately 10 and 20-fold induction of phospho-Smc1 (pS957) and phospho-Smc1 (pS966), respectively, in C57 b16 mice exposed to 10 Gy TBI. Furthermore, the ELISAs detected induced phospho-Smc1 (pS957) and phospho-Smc1 (pS966) levels from 2 to 5-fold in NSG1 and NSG2 mice exposed to 2.75 Gy TBI. These data indicate that the ELISAs described above are able to detect Smc1 phosphorylation in circulating mammalian cells after a in vivo TBI exposure within a physiologically relevant range.

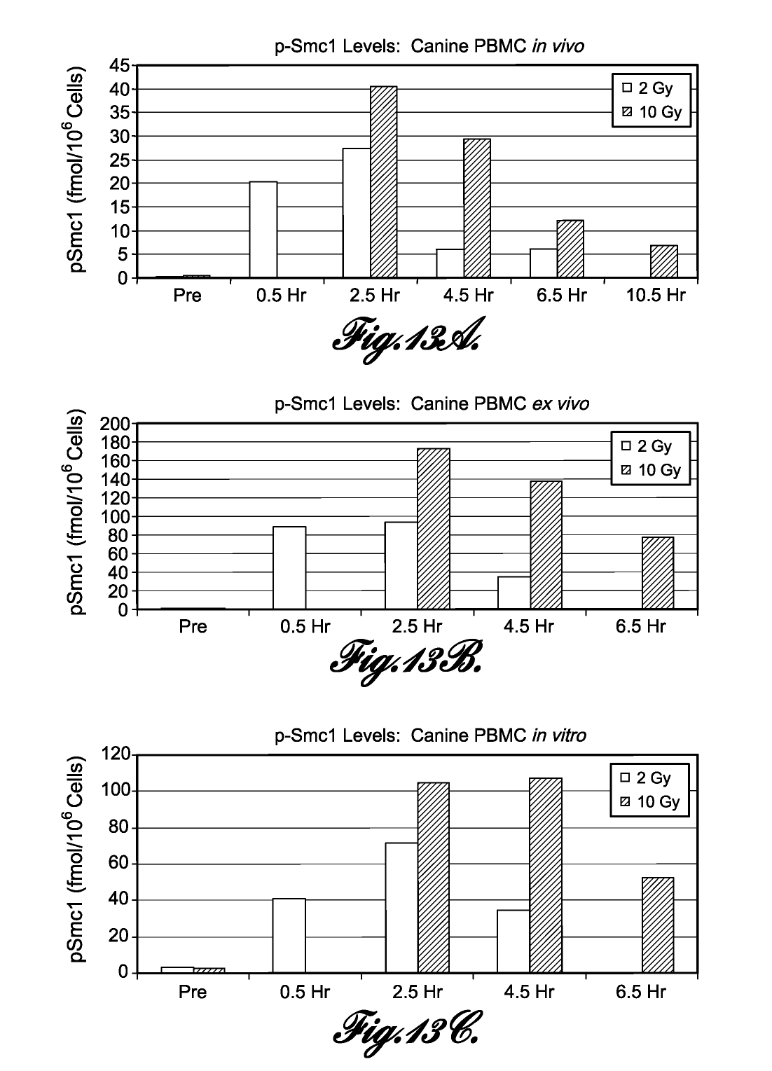

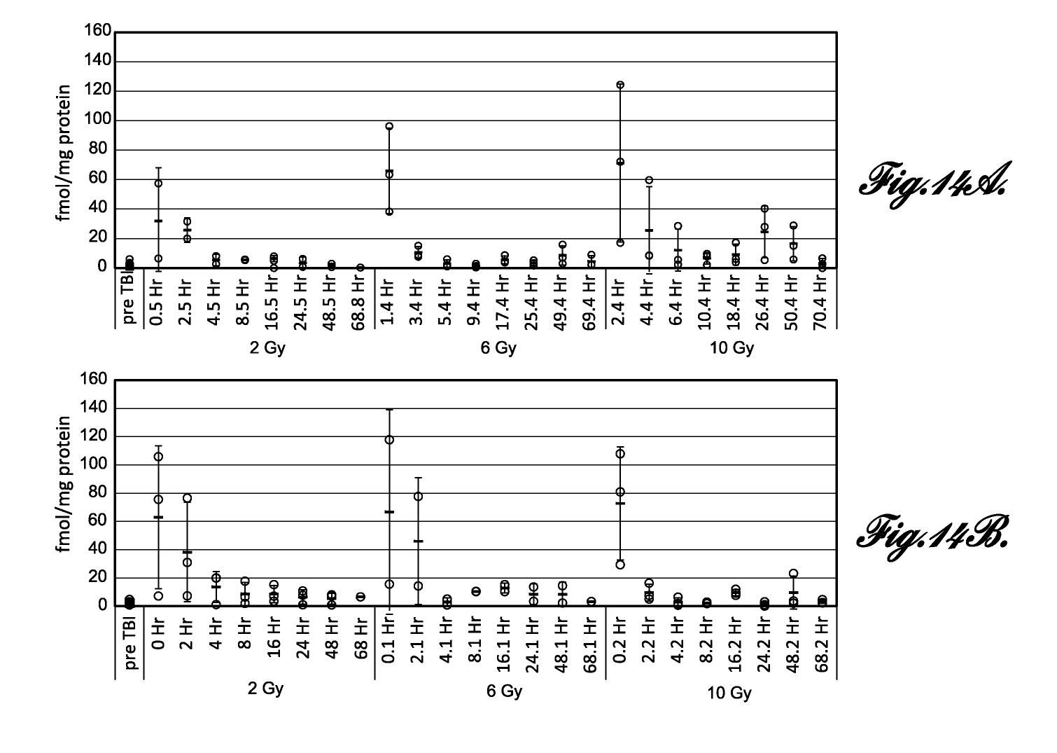

Regarding the canine models, representative results are shown in FIG. 13. Most significantly, the phospho-Smc1 (pS957) ELISA revealed a significant time- and dose-dependent induction of phospho-Smc1 (pS957) in blood samples obtained post-TBI. The data demonstrate that IR-induced proteomic changes can be detected in circulating cells after TBI in the well-established canine radiation model, and support the feasibility of using the proteome, as monitored by ELISA, for biodosimetry. As shown in FIG. 13A, the in vivo response of phospho-Smc1 (pS957) induction peaked between 2.5-4.5 hours post-TBI exposure. The phospho-Smc1 (pS957) signal persists at least 6.5 hours after an exposure of 2 Gy and greater than 10.5 hours after 10 Gy exposure. Both ex vivo (FIG. 13B) and in vitro (FIG. 13C) exposures also resulted in time- and dose-dependent induction of phospho-Smc1 (pS957), interestingly to even higher levels than in vivo. The reason for this higher response is not known.

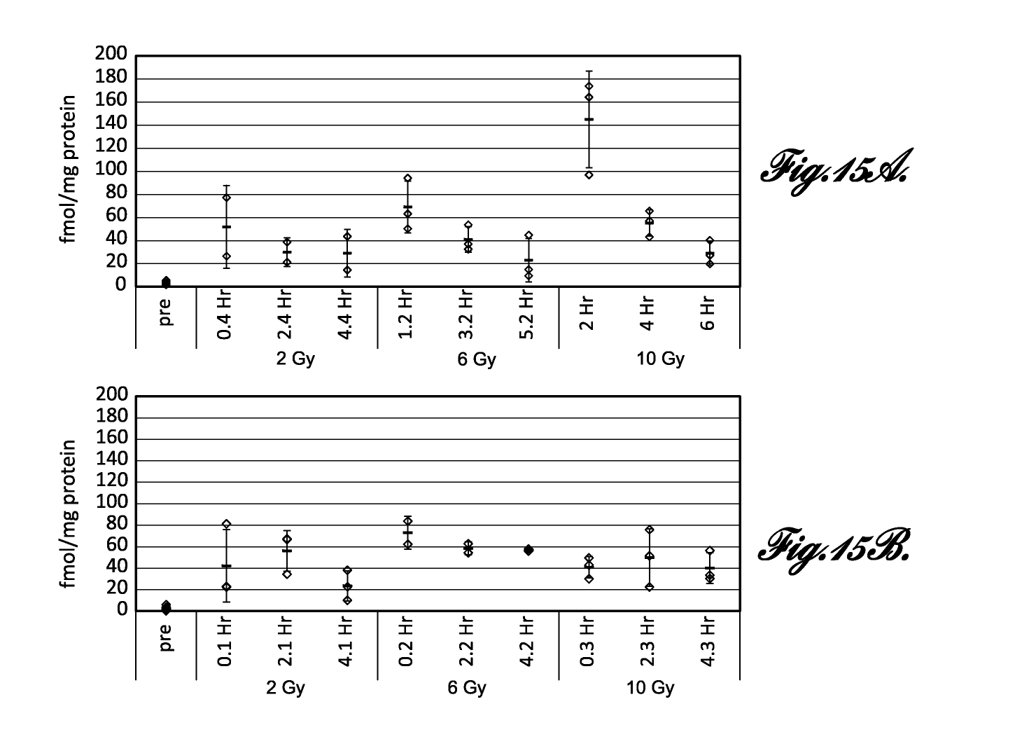

FIGS. 14-16 illustrate expanded data sets for the canine in vivo, ex vivo and in vitro assays described above and illustrated in FIG. 13.

FIG. 14 illustrates the levels of phospho-Smc1 (pS957) levels in animals receiving TBI at different rates. Animals received either 2, 6, or 10 Gy of TBI at either 7 cGy per minute (FIG. 14A) or 70 cGy per minute (FIG. 14B). Blood samples were drawn and cells were isolated by Ficoll gradient and lysates prepared at the indicated times relative to the start of TBI. Phospho-Smc1 (pS957) levels in cell lysates were measured in at least duplicate by ELISA. Circles represent the mean measurement value for each animal while the bar represents the mean value for the three animals for a given treatment. The error bars represent the standard deviation of the mean for the animals in a treatment. The illustrated data confirm a significant time- and dose-dependent induction of phospho-Smc1 (pS957) in blood samples obtained post-TBI. The initial peak of Smc1 phosphorylation at S957 occurs around 2 hours post exposure, with higher peaks for higher doses. It is noted, however, that elevated levels of Smc1 phosphorylation at S957 are detectable at least as far as 50 hours post TBI, indicating a long “tail” to the Smc1 phosphorylation response to IR. Furthermore, slightly higher rates of exposure (i.e., 70 cGy per minute versus 7 cGy per minute) result in slightly higher peaks.

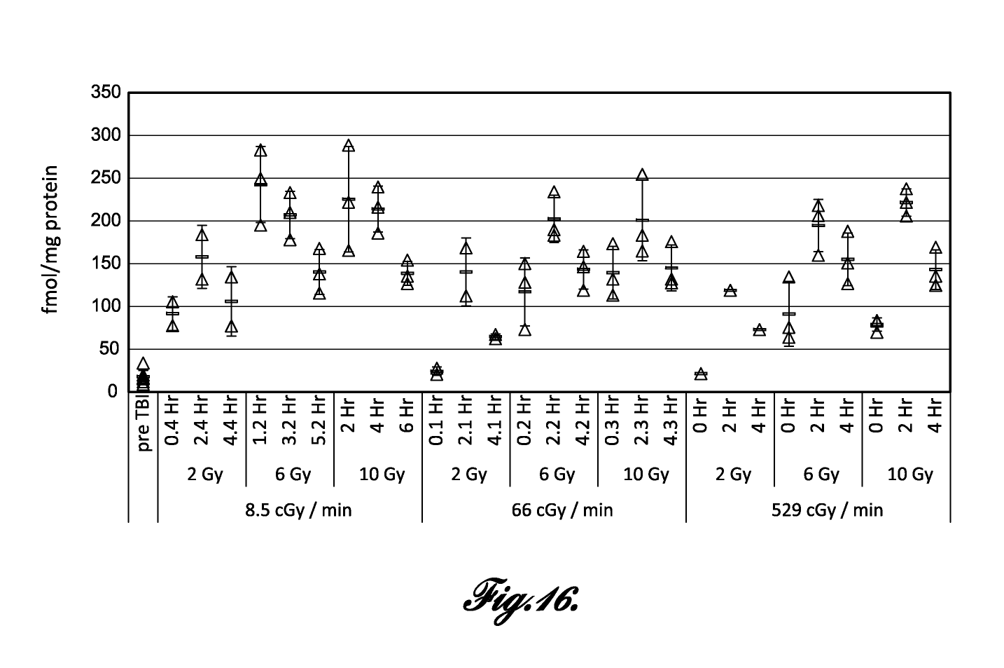

FIG. 15 illustrates the levels of phospho-Smc1 (pS957) levels in cell lysates derived from canine PBMCs exposed ex vivo to IR at different rates. PBMCs were obtained from animals. The whole blood samples received 2, 6, or 10 Gy of ionizing radiation at either 8.5 cGy per minute (FIG. 15A) or 66 cGy per minute (FIG. 15B). Cells were incubated at 37° C. for the indicated times relative to the start of the irradiation. Cells were isolated by Ficoll gradient and lysates were prepared. Phospho-Smc1 (pS957) levels in cell lysates were measured in at least duplicate by ELISA. Open diamonds represent the mean measurement value for each sample while the bar represents the mean value for all samples for a given treatment. The error bars represent the standard deviation of the mean for the animals in a treatment. As illustrated in 15A, ex vivo exposure to ionizing radiation at 8.5 cGy/minute resulted in an initial peak of Smc1 phosphorylation at S957 that occurs around 2 hours post exposure. Furthermore, the induction of Smc1 phosphorylation at S957 was strongly dose-dependent. The data illustrated in FIG. 15B also indicate an early peak of Smc1 phosphorylation at S957, followed by a gradual decline in phosphorylation levels from 2 to 4 hours.

FIG. 16 illustrates the levels of phospho-Smc1 (pS957) levels in cell lysates derived from cultured canine PBMCs irradiated in vitro. Cells were isolated by Ficoll gradient from a pre-TBI blood sample. The cells were activated with 11-2, anti-CD3 and anti-CD28 antibodies and expanded in culture for eight days. Cells were divided into treatment flasks, allowed to equilibrate for 36 to 40 hours and then treated at either 2, 6, or 10 Gy delivered at 8.5, 66, or 529 cGy per minute. Protein lysates were prepared from cells harvested at the indicated times relative to the start of IR treatment. Phospho-Smc1 (pS957) levels in protein lysates were measured in at least duplicate by ELISA. Open triangles represent the mean measurement value for each sample while the bar represents the mean value for all samples for a given treatment. The error bars represent the standard deviation of the mean for the samples in a given treatment. The illustrated data confirm a significant time- and dose-dependent induction of phospho-Smc1 (pS957) in cultured blood cells irradiated in vivo. Consistent with the in vivo and ex vivo data, the initial peak of Smc1 phosphorylation at S957 occurs around 2 hours post-exposure, with higher peaks for higher doses. Interestingly, the higher rates of IR exposure did not result in elevated levels of phospho-Smc1 (pS957).

Conclusion:

In summary, the phospho-Smc1 ELISA assay, developed as described in Example 2, successfully detected phosphorylation of Smc1 in murine blood lysates after total body exposure to ionizing radiation. The phospho-Smc1 ELISA assay also successfully detected the time- and dose-dependent phosphorylation of Smc1 in canine PBMC after exposure to IR in vivo, ex vivo, and in vitro.

### Example 4

This Example demonstrates the use of the phospho-Smc1 ELISA assay, as described in Example 2, to detect phospho-Smc1 induction in human PBMCs exposed to IR ex vivo or in vivo after culture.

Methods and Results:

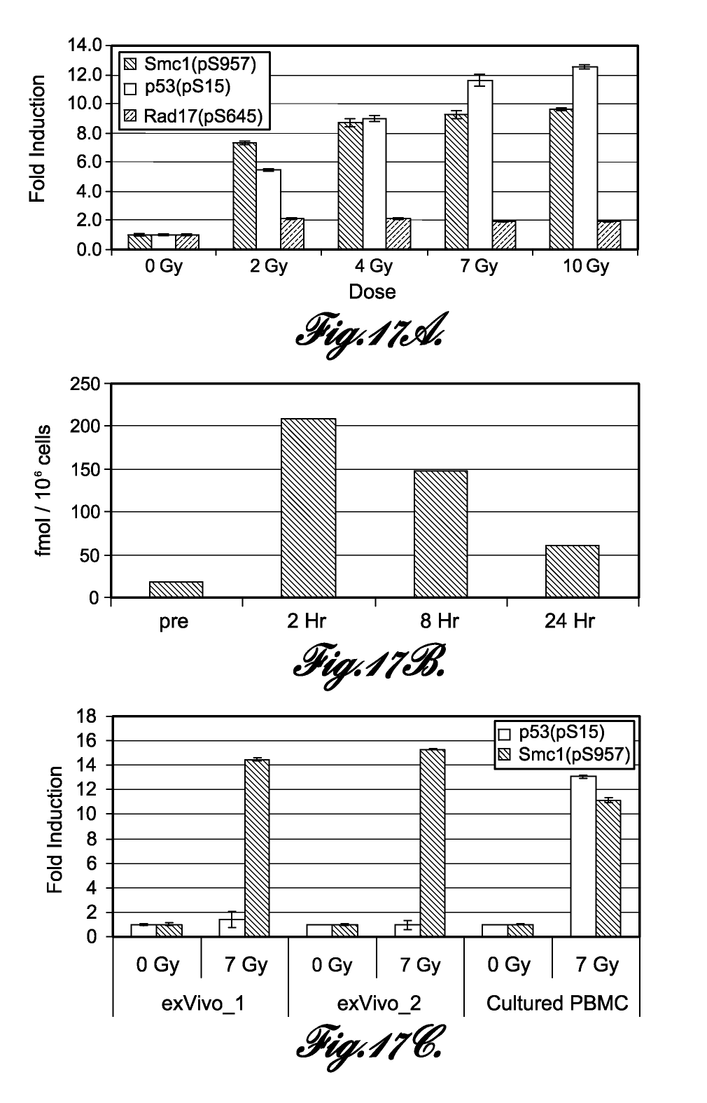

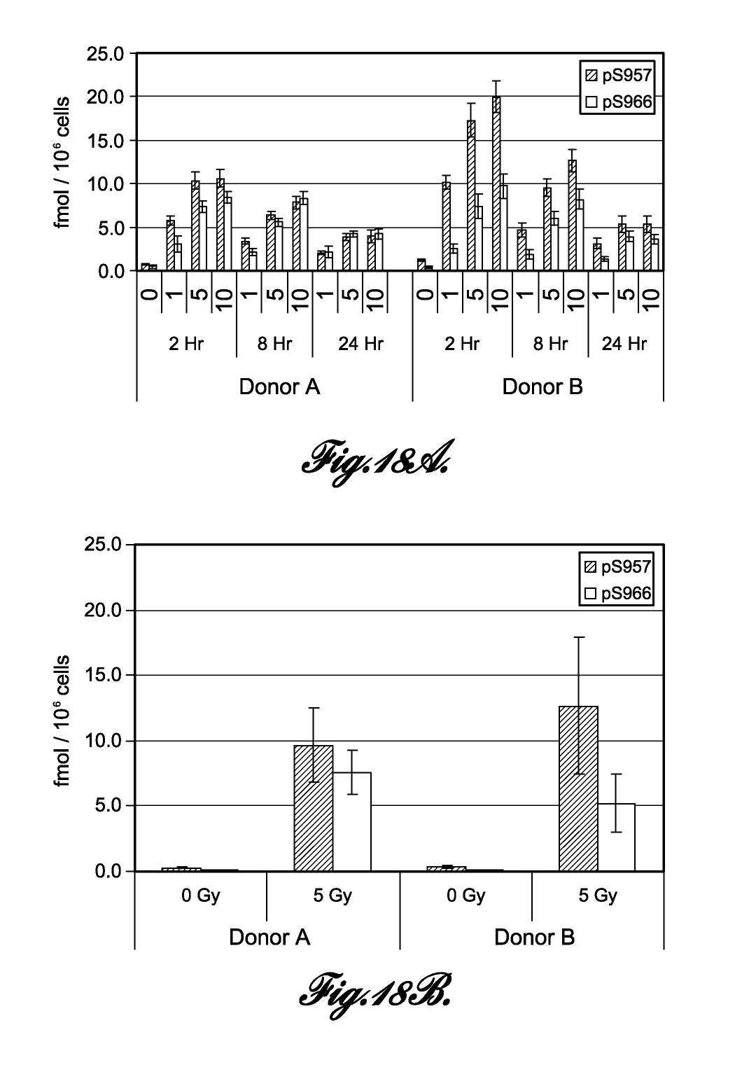

ELISA assays as described above in Example 2 were used to measure dose- and time-dependent phosphorylation of Smc1 at S957 in human PBMCs exposed to IR ex vivo or in vivo after culture. The results are shown in FIG. 17. Also illustrated are results from similar assays used to measure induction of p-53 and p-Rad17. Regarding cultured PBMCs, human PBMCs were isolated from a normal blood donor by Ficoll gradient, activated with α-CD3/CD28 antibodies+IL-2, cultured for 10 days, and exposed to 0, 2, 4, 7, or 10 Gy at 5.5 Gy/min. Lysates were prepared 2 hours post-IR, and phospho-Smc1 (pS957) (and p-53 and p-Rad17) levels were quantified in triplicate using the ELISAs described above. As shown in FIG. 17A, the ELISA assay detected a radiation dose-dependent induction of phospho-Smc1 (pS957) in the isolated and activated human PBMCs.

Additionally, as shown in FIG. 17B, the ELISA assay detected the time-dependent phosphorylation of Smc1 at 5957 (with regard to time of exposure to radiation). Human PBMCs were isolated from a normal blood donor, activated with α-CD3/CD28 antibodies+IL-2, cultured for 10 days, and exposed to 0 (mock) or 10 Gy at 5.3 Gy/min. Lysates were prepared before IR or 2, 8, or 24 hours post-IR, and phospho-Smc1 (pS957) was quantified in triplicate on two independent plates using the ELISA assay described above. As shown in FIG. 17B, the amount of phosphorylated Smc1 peaked at 2 hours post IR-exposure, and gradually decreased to yet elevated levels by 24 hours post-IR exposure.

Regarding PBMCs exposed ex vivo, human whole blood samples obtained from two independent donors (“exVivo_{—}1”, “exVivo_{—}2”) were exposed ex vivo to 0 or 7 Gy at 5.5 Gy/min. At 2 hours post-IR, PBMC were isolated, and protein lysates were analyzed by ELISA. For comparison, PBMC from a third donor (“Cultured PBMC”) were activated with α-CD3/CD28 antibodies+IL-2, cultured for 10 days, and exposed to 0 or 7 Gy. All lysates were prepared 2 hours post-IR, and phospho-Smc1 (pS957) and p-53 levels were quantified in triplicate using the ELISAs described above. The induction of phospho-Smc1 (pS957) and p-53 were normalized to pre-exposure levels. As shown in FIG. 17C, the ELISA assay detected the induction of phospho-Smc1 in whole blood samples exposed to IR ex vivo.

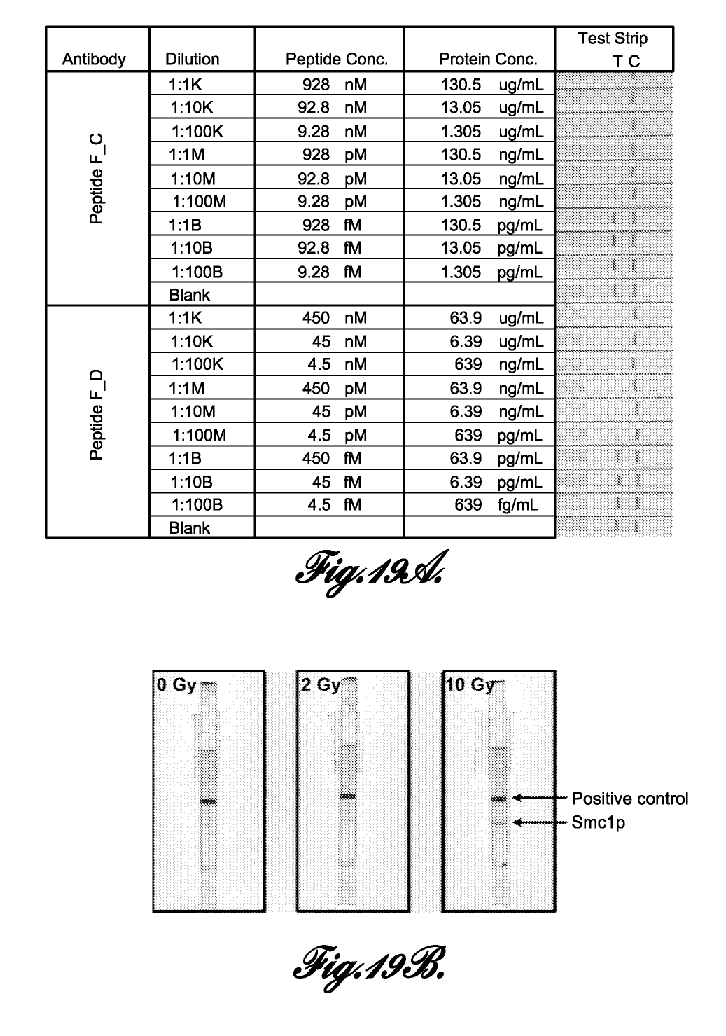

FIG. 18 illustrates a data set that is supplementary to the data illustrated in FIG. 17, and confirms the ability of the ELISA to detect phosphorylation of the Smc1 protein at S957 and 5966 in human PBMC exposed to IR ex vivo or in vitro after culture.

After informed consent was obtained, blood was collected by phlebotomy from a healthy 31-year-old male donor (donor A) on three different occasions over 1 month (dates of collection were February 1, February 16 and March 2). A second set of blood samples was collected from blood from a second donor (a healthy 24-year-old male, donor B) that was drawn on three different occasions over 5 weeks (dates of collection were March 29, April 20, and May 3).

At the time of each of the three collections, seven independent aliquots of blood were prepared. Three 10-ml aliquots were used for technical replicates to examine the response of cycling human PBMCs (in vitro). Specifically, PBMCs were isolated by Ficoll gradient, and the cells were placed in culture and activated with anti-CD3/28 antibodies plus IL-2. Cells were cultured for 8 days and then split into treatment flasks and grown for an additional 2 days. Cells were either mock irradiated (0 Gy) or exposed to 1, 5 or 10 Gy and returned to the incubator. Cells were harvested at 2, 8 and 24 hours post-irradiation, and protein lysates were prepared from the cells and evaluated by ELISA in duplicate on two independent ELISA plates. The mean concentrations of p-Smc1 (pS957) and p-Smc1 (pS966) were calculated from the standard peptide curve and the values were normalized to cell count. The means±SD of the values of all measurements for all technical replicates from all three blood draws were plotted.

In parallel, four 5-ml aliquots of blood were used to examine the response of noncycling human PBMCs (ex vivo). Specifically, two of the blood aliquots were mock-irradiated (0 Gy), two aliquots were exposed to 5 Gy, and blood was incubated at 37° C., 95% air/5% CO2 for 2 hours, at which time PBMCs were isolated by Ficoll gradient, and protein lysates were prepared from the cells and evaluated by ELISA in duplicate on two independent ELISA plates. The mean concentrations of phospho-Smc1 (pS957) and phospho-Smc1 (pS966) were calculated from the standard peptide curves, and the values were normalized to cell count. The mean concentration±SD for all technical replicates from the three blood draws were plotted.

For the cycling cells (i.e., in vitro PBMCs), a maximum induction of phospho-Smc1 (pS957) and phospho-Smc1 (pS966) was observed by 2 hours post-irradiation (FIG. 18A). At 2 hours, the 5- and 10-Gy levels were within one standard deviation of each other, suggesting that the sites were nearing saturation at these doses and this time. At 8 hours post-irradiation, there were reduced levels of both analytes relative to 2 hours, but there was a clear separation of all three doses. At 24 hours post-irradiation, both phospho-Smc1 (pS957) and phospho-Smc1 (pS966) levels remained elevated; for example, the signals in the 1-Gy samples were three to four times higher than that in the mock-irradiated samples. For the quiescent, or non-cycling cells (i.e., ex vivo PBMCs) (FIG. 18B), an induction was observed of both phospho-Smc1 (pS957) and phospho-Smc1 (pS966) to levels comparable to those observed in the genetically identical cycling cells at the same dose and time (5 Gy, 2 h) (FIG. 18A).

Conclusion:

In summary, the phospho-Smc1 ELISA assays described herein successfully detected time- and dose-dependent phosphorylation of Smc1 in human PBMCs, when exposed to IR within a physiologically relevant dose range after culture or in whole blood samples ex vivo.

### Example 5

This Example demonstrates the use of the phospho-Smc1 ELISA to detect the phosphorylation of Smc1 in human PBMCs after in vivo exposure to IR.

Methods:

The ELISA assay, developed as described in Example 2, was used to detect phosphorylation of Smc1 in human blood samples from a set of patients including individuals receiving a variety of radiation therapies for cancer. Required IRB approvals for human testing and the informed consent was obtained. Five patients were enrolled in the study. Two of the patients received total body irradiation (TBI) as part of their conditioning regimen for bone marrow transplantation. Another 2 patients received partial body irradiation as treatment for prostate cancers. The 5th patient received an infusion of ¹³¹-Iodine (coupled to an anti-CD20 antibody) as radioimmunotherapy for a Diffuse Large B cell Lymphoma.

Results:

For each patient, blood was collected pre-treatment as well as at one or more times post-treatment. Baseline levels of phospho-Smc1 were established in the pre-treatment samples, and induced levels of phospho-Smc1 were established in the post-treatment samples, allowing the fold induction to be calculated. All three types of exposure (TBI, partial body, and infusional) resulted in induction of phospho-Smc1. This data is incorporated into the expanded data set described below in Example 7, and illustrated in FIGS. 21-25.

Exposure to total body radiation resulted in the highest detected induction of phospho-Smc1.

A patient with acute myeloid leukemia undergoing conditioning for bone marrow transplantation received a 1.5 Gy fraction of total body irradiation (TBI). Blood samples were obtained from the patient pre-TBI and at 3 and 6 hours post-TBI. Fold induction of phospho-Smc1 was determined in isolated PBMC using the ELISA assay described above. Levels of phosphorylated Smc1 peaked at 3 hours post-TBI exposure with about 25-fold more compared to pre-exposure levels.

A Hodgkin's lymphoma patient undergoing conditioning for bone marrow transplantation received a 1.5 Gy fraction of TBI prior to bone marrow transplantation. Blood samples were obtained from the patient pre-TBI and at 3 and 6 hours post-TBI. Fold induction of phospho-Smc1 was determined in isolated PBMC using the ELISA assay described above. Levels of phosphorylated Smc1 peaked at 3 hours post-TBI exposure with about 28-fold more compared to pre-exposure levels.

Partial-body exposures to IR also resulted in the induction of phospho-Smc1.

A first prostate cancer patient received the prescribed dose of 180 cGy delivered to the clinical target volume consisting of pelvic lymph nodes, prostate and seminal vesicles. Treatment was carried out on a Varian Clinac CD linear accelerator using 7 fields for step-and-shoot intensity modulated radiotherapy (IMRT). Blood samples were collected from the patient pre- and 5 hours post-radiation therapy (XRT), PBMCs were isolated and levels of phospho-Smc1 were determined using the ELISA assay. Phosphorylated Smc1 was induced almost 4-fold after IR exposure.

A second prostate cancer patient received the prescribed dose of 223 cGy delivered to the clinical target volume consisting of the prostate only, using 7 field step-and-shoot IMRT, carried out on a Varian 21EX linear accelerator. Blood samples were collected from the patient pre- and at 5 hours post-XRT, PBMCs were isolated and levels of phospho-Smc1 were determined using the ELISA assay. Phosphorylated Smc1 was induced more than 5-fold after IR exposure.

Induction of phospho-Smc1 was also detected in a patient receiving an infusion of ¹³¹-Iodine coupled to an anti-CD20 antibody.

A patient with Diffuse Large B cell Lymphoma was treated with radioimmunotherapy by infusion of 592 mCi of Iodine-¹³¹-labeled anti-CD20 antibody. Blood samples were collected from the patient pre- and 24 hours post-infusion of the radioisotope, PBMCs were isolated and levels of phospho-Smc1 were determined using the ELISA assay. Phosphorylated Smc1 was induced almost 4-fold at 24 hours after IR exposure.

It is noted that the time points of blood collection post-treatment are approximate and were constrained by patient availability and clinic workflow, and the doses, dose rates, and volumes exposed were of course dictated by a patient's prescribed treatment protocol. Hence, unavoidable differences in these parameters amongst these 5 patients make it difficult to rigorously compare the relationship between in vivo exposures and fold induction at this early point. Nonetheless, the finding that phospho-Smc1 induction was higher following TBI (25-30 fold at the maximal measured level) compared to partial body exposure (4-5 fold) is likely to reflect the dosimetric response of phospho-Smc1 to ionizing radiation. For external beam exposures, the in vivo PBMC phospho-Smc1 response peaks sometime before 6 hours, but is still present at 6 hours.

In summary, the phospho-Smc1 ELISA successfully detected phosphorylation of Smc1 in human PBMCs after exposure to IR in vivo as part a variety of cancer therapy regimens. The data described in this Example provide critical demonstration of the feasibility of using the phospho-Smc1 ELISA for point-of-care detection of radiation exposure victims.

### Example 6

This Example demonstrates the development of a lateral flow assay based on the ELISA technology described above for point of care diagnosis.

Methods:

To assess feasibility of converting the phospho-Smc1 ELISA into a point of care (POC) lateral flow assay, the monoclonal antibodies (mAbs) and synthetic phospho-protein reference controls, described above in Example 2, were integrated into the C-FLAT rapid format assay (BioAssay Works®, Ijamsville, Md.) and exposed to the various concentrations of antigen. Phospho-Smc1 capture mAb was attached to the test strip, and the detection mAb was coupled to colloidal gold nanoparticles. Control phospho-Smc1 peptide phospho-antigen (F_C or F_D; 2 different antigens) was added to the sample at various concentrations (1.305 pg/mL to 130.5 μg/mL for antigen F_C, and 639 fg/mL to 63.9 μg/mL for antigen F_D). As shown in FIG. 19A, signal intensity of the test line varied as expected with target concentration, demonstrating that the assay reagents are active and compatible with the lateral flow test format. The test line is indicated in FIG. 19A with a (T) and the positive control line is indicated with a (C). The sensitivity of the phospho-Smc1 lateral flow assays are within 10-fold of the best of the ELISA assays described above, demonstrating their compatibility for development into a POC diagnostic that is capable of providing an indication of degree of IR exposure, in addition to a binary “exposed/not exposed” diagnosis.

The lateral flow assay format was assessed for the ability to detect IR-induced induction of phospho-Smc1 (S957) in human cells. Human LBL cells were exposed to 0, 2, or 10 Gy of IR at 5.3 Gy/min. Protein lysates were prepared 2 hrs post-IR and analyzed in a lateral flow assay using the ELISA antibodies, namely the Smc1 capture antibodies and phospho-Smc1 (S957) antibodies. Referring to FIG. 19B, the upper reactive band is the positive control (goat anti-rabbit Ab), and the lower IR-dependent reactive band detects phospho-Smc1. The phospho-Smc1 signal is strong and dose-dependent.

In summary, the reagents developed for the phospho-Smc1 ELISA are shown to be compatible with a lateral flow assay format for point of care diagnosis. The phospho-Smc1 lateral flow assay successfully detected the phosphorylation of Smc1 in human LBL cell lysates after IR exposure. The signal was strong and dose-dependent, demonstrating clear feasibility of this format for point of care diagnosis.

To optimize the POC lateral flow format for use with small amounts of biological sample from a subject, leukocytes were first isolated from human whole blood exposed to IR using αCD45 Dynabeads (Invitrogen, Carlsbad, Calif.) before subjected to the lateral flow assay. It is noted that cells present in whole blood that express CD45 on the surface include B cells, T cells, Dendritic cells, NK cells, macrophages/monocytes, stem cell precursor cells and granulocytes. In a first experiment, whole blood from a healthy human donor were exposed to 0 or 8 Gy IR. The cells were incubated for 30 minutes at 37° C. and then divided in to multiple 1 mL samples. Each 1 L irradiated whole blood sample was mixed with 100 μL CD45 Dynabeads solution (Invitrogen, Carlsbad, Calif.) and incubated for 20 minutes at 4° C. A magnet was applied to immobilize the Dynabeads, and CD45 expressing cells attached thereto, and the supernatant was removed. 100 μL lysis buffer was subsequently added to lyse the bead bound cells. A magnet was applied again and the supernatant containing protein lysate was removed. A subsample from the 0 Gy and 8 Gy lysate groups were spiked with 50 fmol of the hybrid standard peptide F_Cp to create a positive test control response for each IR exposure group. The protein lysates were mixed with 10 μL Cp gold particles to which detection mAb specific for phosphorylated Smc1 (pS957) are bound.

The lysate/gold particle mixtures were applied to the lateral flow assay strip (C-FLAT system) as described above, for 20 minutes. As described above in the context of FIG. 19, the test strip contains distinct test and control lines disposed perpendicularly in order along the main axis of the strip. The lysate is delivered to the sample pad, which then migrates by capillary action through the test strip. The lysate first encounters the test line where capture antibody specific for the capture epitope of the Smc1 protein (FHC37_F, Table 4 and FIGS. 2-3) are immobilized on the substrate. Smc1 protein will remain bound in the test line when bound to the capture antibody, whereas the rest of the sample continues to migrate along the strip and eventually to the control line. The control line contains immobilized a Rabbit IgG antibodies that serve to capture gold particle/detection antibody complexes that are not retained at the test line. The signal deriving from the control strip provides a positive control that the lysate/gold particle mix (with the detection mAbs bound to the particles) has migrated along the test strip.

As illustrated in FIG. 20A, a strong pS957 signal was detected using 1 mL of irradiated blood (exposed to 8 Gy IR over 30 minutes) wherein the leukocytes were first isolated from the whole blood sample using 100 μL CD45 Dynabeads. As expected, the samples that also included a spike of F_Cp hybrid peptide resulted in strong positive pS957 signals, as did the control lines for all strips.

In a second experiment, lower volumes of whole blood samples and CD45 Dynabeads were used to establish the feasibility of the approach for “finger prick” amounts of blood sample. Whole blood from a normal human donor was divided into 100 μL and 250 μL samples. After exposure to 0 or 8 Gy IR over 30 minutes, the samples were mixed with 25 μL CD45 Dynabeads. The samples were processed and applied to the test strip, as described above, except that 25 μL of lysis buffer was used.

As illustrated in FIGS. 20B and 20C, detectable signals were present for both starting whole blood sample sizes that were exposed to 8 Gy. Again, as expected, the samples that also included a spike of F_Cp hybrid peptide resulted in strong positive pS957 signals, as did the control lines for all strips. This preliminary data indicates that the test strip format is amenable to detection of Smc1 phosphorylation in blood leukocytes after exposure of the blood to a physiologically relevant dose of IR. Future experiments be directed to optimizing the approach to enhance the sensitivity of the assay in context of small initial blood samples.

Conclusion:

As described above, the compatibility of the novel ELISA reagents were demonstrated with the C-FLAT rapid format assay. As shown in FIG. 19A, the Smc1 capture antibodies and phospho-Smc1 (S957) detection antibodies perform well in the lateral flow assay format. Further, these results demonstrate the sensitivity of the lateral flow assays as comparable to the best of ELISA assays, indicting that the assays may be useful to ascertain the degree of exposure, in addition to providing a binary “exposed/not exposed” diagnosis. Finally, the data demonstrate the feasibility of the lateral flow format assay using the ELISA reagents for POC end use with small blood input samples.

### Example 7

This Example describes the expanded use of the phospho-Smc1 ELISA assays to monitor phospho-Smc1 induction in human PBMCs during and after in vivo exposures that occur as part of cancer treatment. The data described herein is from an ongoing study initially described in Example 5. This expanded data set incorporates the data described in Example 5. This example provides descriptions of the ongoing data set as it has been updated. However, only the more recent figures illustrating the aggregate data are included.

Initial Methods:

The ELISA assay, developed as described in Example 2, was used to detect phosphorylation of Smc1 in human blood samples from patients receiving a variety of radiation therapies for cancer. Required IRB approvals for human testing and the informed consent was obtained. Eight patients received eight exposures of total body irradiation (TBI) over four days as part of their conditioning regimen for bone marrow transplantation. Each exposure was at 1.5 Gy for a cumulative exposure of 12 Gy. Blood was drawn at various times before and during the course of treatment. An additional four patients received a series of partial body IR exposure as treatment for prostate cancer. Each exposure was 1.8 Gy and delivered to the clinical target consisting of pelvic lymph nodes, prostate and seminal vesicles. Blood was drawn before and 2 hours after the initial exposure. One patient received a test and a separate therapeutic infusion of ¹³¹Iodine (coupled to an anti-CD20 antibody) as radioimmunotherapy for a Diffuse Large B Cell Lymphoma. Blood was drawn at various times before and after the infusions.

Initial Results:

For each patient, blood was collected pre-treatment as well as at one or more times post-treatment. Baseline levels of phospho-Smc1 were established in the pre-treatment samples, and induced levels of phospho-Smc1 were established in the post-treatment samples. Consistent with the results presented in Example 5, all three types of exposure (TBI, partial body, and infusional) resulted in induction of phospho-Smc1.

Exposure to repeated total body radiation resulted in induction and maintenance of phospho-Smc1 levels.

Eight patients with acute myeloid leukemia undergoing conditioning for bone marrow transplantation received a series of 12 exposures of total body irradiation (TBI), over four days. Each exposure consisted of a 1.5 Gy fraction, totaling 12 cumulative Gy. Blood was drawn from at six time points during the course of treatment: before and at approximately 2, 8, 32, 56, and 80 hours after the initial TBI.

Initially, levels of phospho-Smc1 were determined in isolated PBMC obtained pre-TBI and at 2 and 8 hours post-TBI using the ELISA assay targeting Smc1 pS957. Mean levels of phosphorylated Smc1 peaked at 2 hours post-TBI exposure and demonstrated a slight decrease by 8 hours post-TBI.

Next, levels of phospho-Smc1 were determined in isolated PBMC obtained 32, 56, and 80 hours post-TBI using the ELISA assay targeting Smc1 pS957, in addition to the PBMCs obtained pre-TBI and at 2 and 8 hours post-TBI as described above. Mean levels of phosphorylated Smc1 peaked at 2 hours post-TBI exposure and slightly decreased over the remaining time points.

Subsequently, levels of phospho-Smc1 were determined in isolated PBMC obtained pre-TBI and at 2, 8, 32, 56, and 80 hours post-TBI using independent ELISA assays targeting Smc1 pS966, in addition to the levels of Smc1 pS957 described above. The available data indicates that the ELISAs specific to Smc1 pS957 and Smc1 pS966 are both capable of detecting phosphorylated Smc1 induced by repeated TBI exposures to humans. Based on the number of patients in the trial, a rigorous comparison between the phosphorylated markers is not possible. However, based on three patients, the data indicate that the Smc1 pS957 ELISA reveals a higher level of phosphorylation in response to TBI.

Partial-body exposures to IR resulted in the induction of phospho-Smc1.

Four prostate cancer patients received a series of prescribed partial body doses of X-ray therapy (XRT). Doses of approximately 1.8 Gy were delivered to the clinical target volume consisting of pelvic lymph nodes, prostate and seminal vesicles. Treatment was carried out on a Varian Clinac CD linear accelerator using 7 fields for step-and-shoot intensity modulated radiotherapy (IMRT). Blood samples were collected from the patients pre- and 2 hours post XRT. Peripheral blood mononuclear cells were isolated and levels of phospho-Smc1 were determined using the ELISA assays (pS957 and pS966).

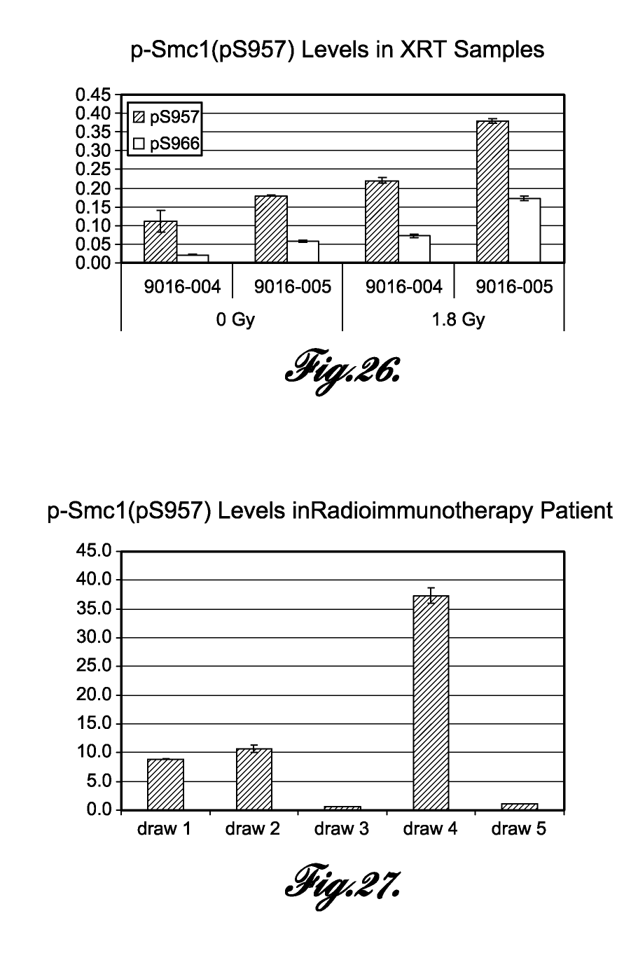

Referring to FIG. 25, levels of phospho-Smc1 were determined in isolated PBMC obtained pre-XRT and at 2 hours post-XRT using the ELISA assay targeting phospho-Smc1 (pS957). Panel A illustrates the specific levels of phosphorylated Smc1 (pS957) for four patients, and Panel B illustrates the mean levels of phosphorylated Smc1 (pS957) across the four patients indicated in Panel A. Levels of phosphorylated Smc1 increased for all four patients at 2 hours post-XRT compared to pre-exposure levels.

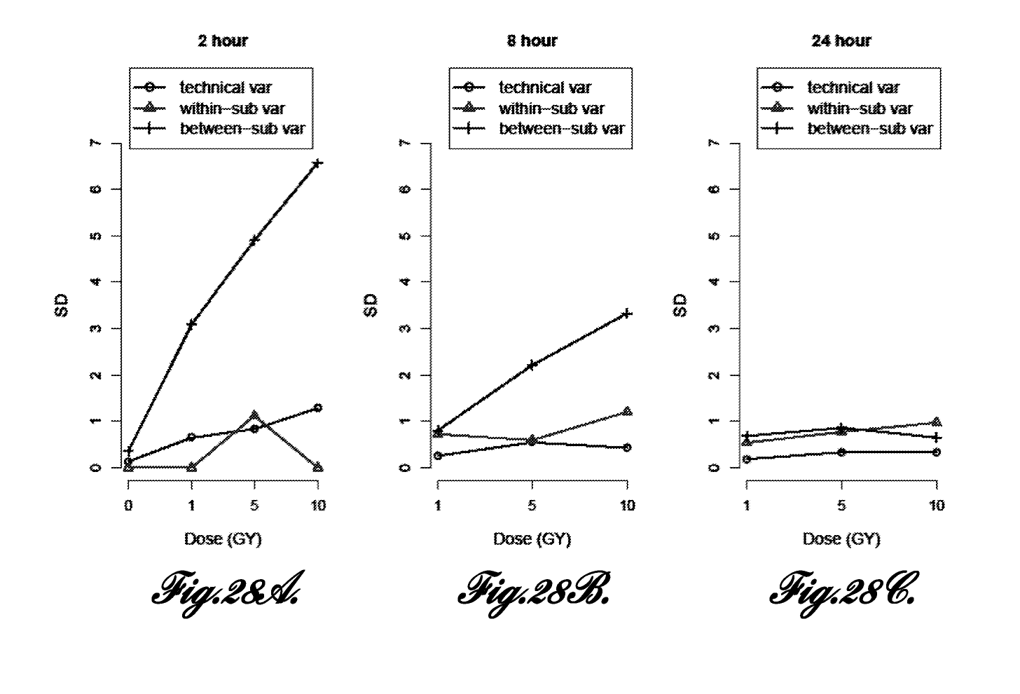

Referring to FIG. 26, levels of phospho-Smc1 were determined in isolated PBMC obtained pre-XRT and at 2 hours post-XRT using independent ELISA assays targeting phospho-Smc1 (pS957) and phospho-Smc1 (pS966). Available data for two patients is shown comparing the pS957 and pS966 ELISAs pre- and post-XRT. Both ELISAs detect an increase in the levels of phosphorylated Smc1. However, the available data indicate that the pS957 ELISA reveals a higher level of phosphorylation in response to partial body irradiation.

Induction of phospho-Smc1 was also detected in a patient receiving an infusion of ¹³¹Iodine coupled to an anti-CD20 antibody.

Referring to FIG. 27, a patient with Diffuse Large B cell Lymphoma was treated with radioimmunotherapy by infusion of ¹³¹Iodine-labeled anti-CD20 antibody. A preliminary test dose of 10 mCi was administered at day −12. At day 0, a therapy dose of 592 mCi was administered. Five blood draws were collected from the patient: draw 1 at day −13 (pre-infusion), draw 2 at day −9 (3 days post-test infusion), draw 3 at day −1 (11 days post-test infusion, 1 day pre-therapy infusion), draw 4 at day +1 (1 day post-therapy infusion), and draw 5 day +8 (8 days post infusion). Peripheral blood mononuclear cells were isolated and levels of phospho-Smc1 were determined using the ELISA assay targeting Smc1 pS957. Twenty-three hours after the administration of the therapy dose (day +1), there was a 49-fold induction of phospho-Smc1 (pS957) relative to the pre-therapy level (FIG. 27). At day +8, phospho-Smc1 induction had decreased to 1.9-fold. The high level of phospho-Smc1 (pS957) in the circulating blood cells 23 hours after the initial exposure is probably the result of the continuous activation of the DNA damage response (DDR) network in response to the injected radioisotope.

In summary, the phospho-Smc1 ELISAs targeting Smc1 pS957 and pS966 successfully detected phosphorylation of Smc1 in human PBMCs after exposure to IR in vivo as part a variety of cancer therapy regimens, including total body exposure, partial body X-ray exposure, and infusion of ¹³¹Iodine. The data described in this Example provide critical demonstration of the feasibility of using the phospho-Smc1 ELISAs for point-of-care detection of radiation exposure victims.

Update:

An updated data set reflects the addition new patients, and new data from the patients previously described regarding additional time points and detected levels of phosphorylated Smc1 for pS966, in addition to pS957.

The updated data set reflects blood samples from a total of 16 cancer patients. Three types of radiation exposure were investigated: TBI (10 patients), partial body irradiation (5 patients), and internal exposure to a radioisotope (¹³¹I) (one patient). Where possible, complete and differential blood counts were obtained from patients within 14 days of their radiotherapy to a confirm that none of the patients had a significant burden of tumor cells in the circulation.

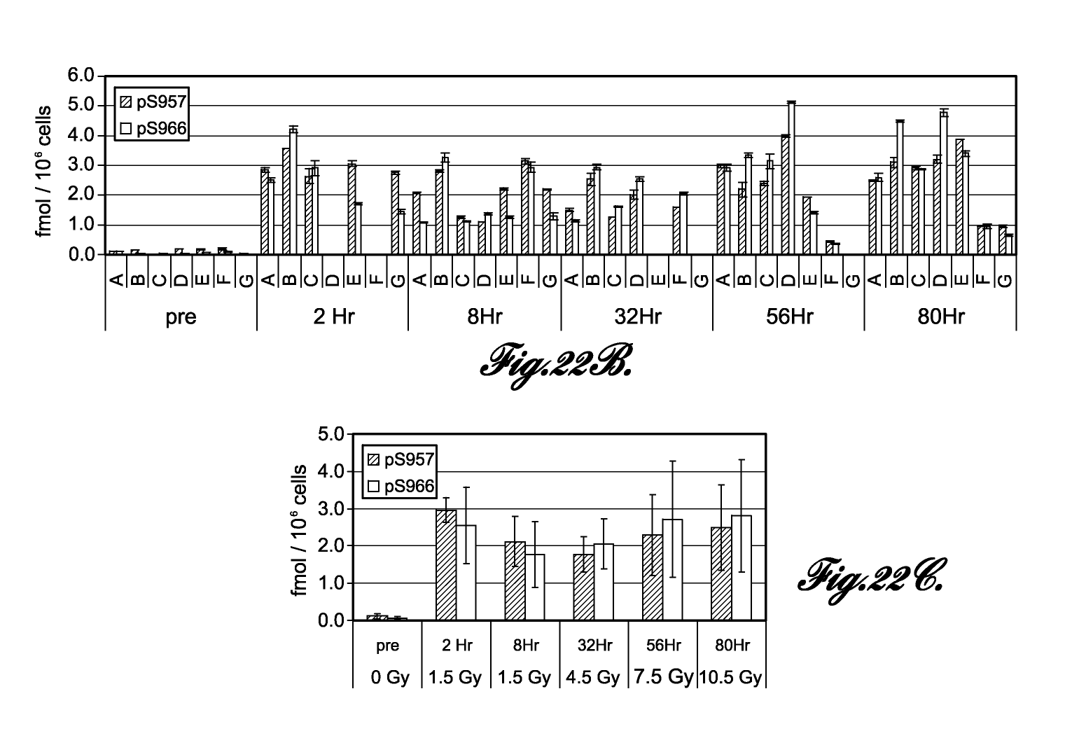

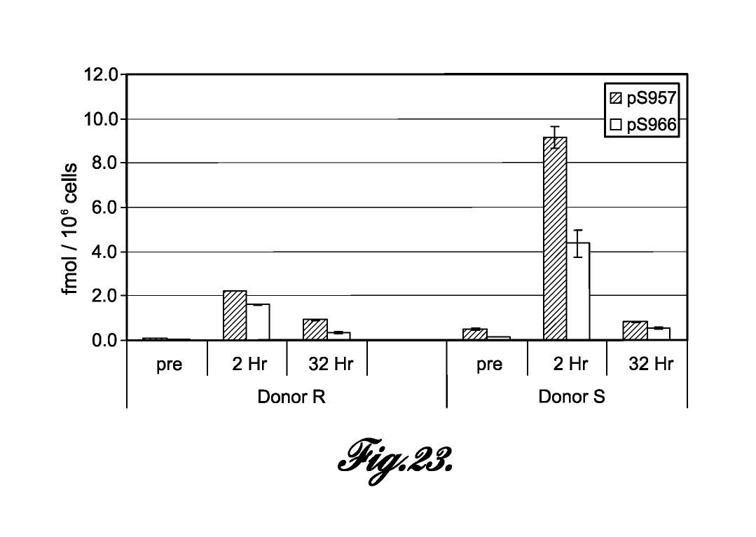

A total of seven patients received TBI as part of their conditioning regimen for cell transplantation therapy. These patients received 1.5-Gy fractions twice daily for four days. Pretreatment blood samples were obtained an average of 5 days (range 1 to 15 days) prior to the first fraction. Post-treatment blood samples were drawn at multiple times (approximately 2, 8, 32, 56 and 80 hours) after the first fraction. See FIG. 22A. The later blood draws occurred after additional fractions of radiation had been delivered. PBMCs were isolated from whole blood samples by RBC lysis and analyzed using the phospho-Smc1 (pS957) and phospho-Smc1 (pS966) ELISAs in triplicate. As illustrated in FIG. 22B, all patients showed significant induction of both phospho-Smc1 (pS957) and phospho-Smc1 (pS966) in their circulating cells after therapeutic radiation exposures. The average induction levels across all patients at 2 hours after exposure were 23-fold for phospho-Smc1 (pS957) and 34-fold for phospho-Smc1 (pS966). These levels of induction in vivo are comparable (i.e., within a factor of two- to three-fold) to those observed in primary human PBMCs after irradiation (see FIG. 18A). As illustrated in FIG. 22C, overall there was a slight increase, relative to the 8 hour time point, in the mean phospho-Smc1 levels across all patients after additional doses of radiation.

In the seven TBI patients described above, the kinetics of the phospho-Smc1 response is complicated by the delivery of multiple fractions of radiation and the timing of sample collection. In contrast, two additional patients received a single fraction of 2 Gy, allowing the determination of the persistence of phospho-Smc1 induction after a single exposure. As illustrated in FIG. 23, both phospho-Smc1 (pS957) and phospho-Smc1 (pS966) showed significant induction at 2 hour post-irradiation. Furthermore, despite no additional exposure to radiation, both phospho-Smc1 (pS957) and phospho-Smc1 (pS966) levels remained elevated at 32 hours post-irradiation (tenfold and twofold for one donor and fivefold and fourfold for the second donor). Although the response of donor S was significantly greater than that of donor R at 2 hours (FIG. 23), the residual induction of phospho-Smc1 was similar in the two patients at 32 hours.

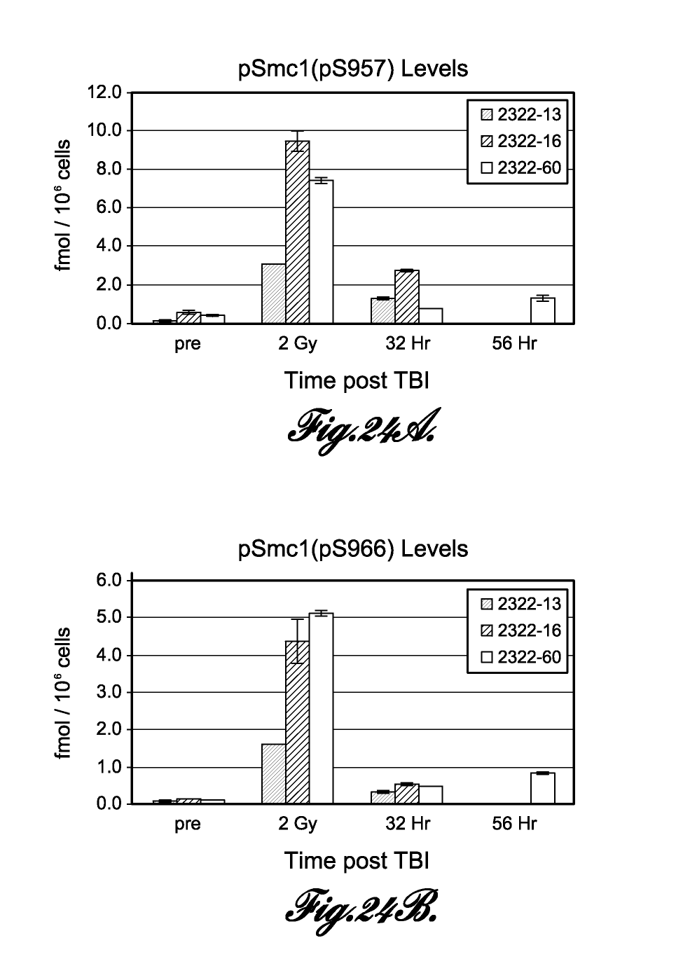

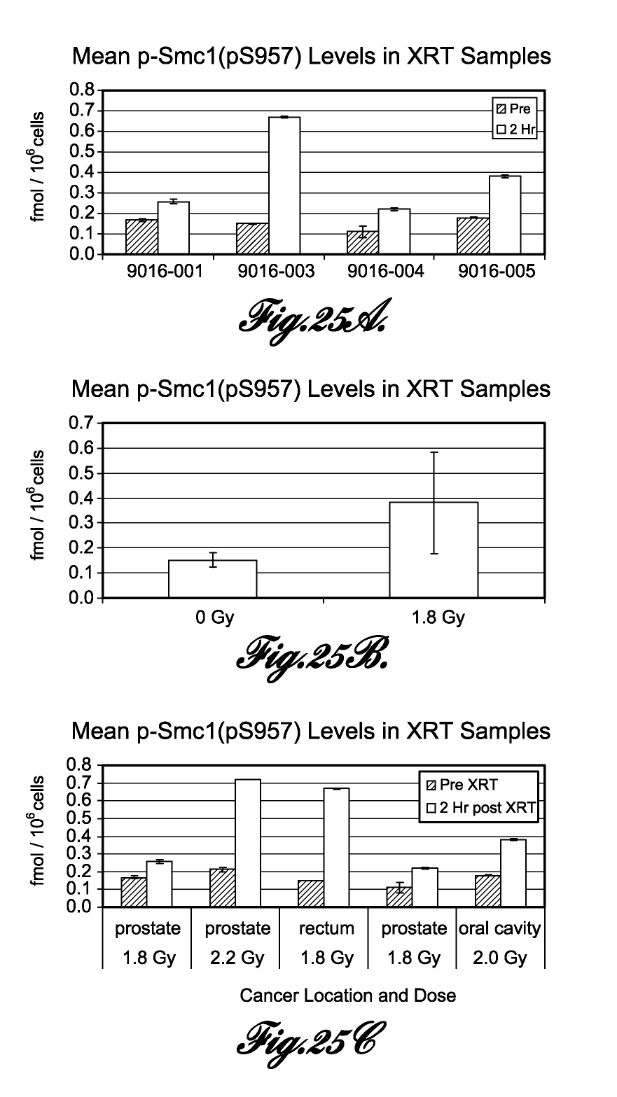

Later, one additional patient receiving the same radiation regimen was added to this data set. Additionally, all three patients were assessed for phospho-Smc1 (pS957) and phospho-Smc1 (pS966) levels at 56 hours after the single dose of 2 Gy TBI, in addition to the 2 hour and 32 hour time points. As above, PBMCs were isolated from the whole blood by RBC lysis and lysates were evaluated by ELISA (in triplicate). As illustrated in FIGS. 24A and 24B, the third (additional) patient had an initial response similar to Donor S of FIG. 23, but also demonstrated similar residual level of Smc1 phosphorylation at 32 hours post irradiation. Interestingly, the additional patient still exhibited residual phosphorylation at 56 hours post-irradiation.

A total of five patients received partial-body irradiation as part of their treatment regimen for solid tumors of either the prostate, rectum or oral cavity. All five patients received a single fraction (ranging from 1.8-2.23 Gy) of radiation each day for a total of 25-35 fractions. Pretreatment blood samples were obtained immediately prior to the first fraction, and a second blood sample was obtained approximately 2 hours after the first fraction had been delivered. PBMCs were isolated from whole blood samples by RBC lysis and analyzed by the phospho-Smc1 (pS957) ELISA in triplicate. As illustrated in FIG. 25C, all five patients showed significant induction of phospho-Smc1 (pS957), with an average induction across all five patients of 2.6-fold. This was significantly less than the induction level seen after TBI (24-fold), likely due to the more restricted radiation field compared to that for the TBI patients. Additionally, the induction level varied significantly among the patients (ranging from 2.0- to 4.5-fold induction), likely due to a combination of interindividual variation as well as differences among patients in the volume of tissue irradiated and the blood flow through the treatment field.

### Example 8

This Example describes the use of phospho-Smc1 Serine 957 and/or Serine 966 as a biomarker in a biological sample obtained from a subject to determine the inherent radiosensitivity of the subject to ionizing radiation exposure.

Background/Rationale:

The DNA damage response pathway is known to play a key role in cancer, aging and neurodegenerative disease, and it is increasingly becoming a focus of possible treatment strategies (see B. Alberts, Science 325:1319 (2009)). Although there has been significant progress in the mechanistic understanding of DNA damage response processes, few studies have bridged the gap between basic research and application to population studies. Unfortunately, population studies of DNA repair capacity have been hindered by the lack of practical, quantitative tools for measuring the DNA damage response in clinical or epidemiological studies. This unmet need was highlighted at a joint NIH workshop for the Working Group on Integrated Translational Research in DNA Repair (DNA Repair, Amt 6: 145-147 (2007)).

As described in this Example, the phospho-Smc1 ELISAs described herein are useful for clinical and epidemiological studies aimed at characterizing interindividual differences in the cellular DDR and relating these differences to clinically relevant end points such as risk for developing cancer or susceptibility to high-grade toxicity from therapies that induce DNA damage (e.g. radiation therapy, clastogenic chemotherapy).

Smc1 is a particularly attractive target for studying interindividual differences in the DNA damage response. First, Smc1 is the most downstream component of the known ATM-NBS1-BRCA1 signaling pathway, and hence the phosphorylation of Smc1 is dependent on the successful completion of multiple upstream steps in activation of this pathway (Kitagawa R. et al., Cold Spring Harbor Symp. Quant. Biol. 70:99-109 (2005)). Accordingly, phospho-Smc1 has the potential to integrate the functional activity of multiple components of the ATM pathway, thereby providing a useful biomarker for detecting human variations in the DNA damage response, as described herein. Second, phospho-Smc1 is induced in response to a wide array of DNA-damaging agents (Yazdi P. T. et al., Genes Dev 16:571-582 (2002); Kitagawa R. et al., Genes Dev 18:1423-1438 (2004); Garg R. et al., Mol Cancer Res 2:362-369 (2004)) and hence its activation is likely to be a general readout of pathway activity. Third, Smc1 phosphorylation is specifically critical for cell survival and maintenance of optimal chromosomal stability after DNA damage, because it is the only target of ATM in which mutation of the phosphorylation sites affects cellular radiosensitivity (Kim S. T. et al., Genes Dev 16:560-570 (2002); Kitagawa R. et al., Genes Dev 18:1423-1438 (2004)). Fourth, despite its critical role in the DDR, there is tremendous human variation in the phosphorylation of Smc1 in response to DNA damage. For example, cells from patients afflicted with the genetic disorder AT are severely defective in Smc1 phosphorylation in response to ionizing radiation due to a lack of ATMp kinase activity, (see FIG. 10).

Methods:

Technical and biological variation of Smc1 phosphorylation was assessed for cultured human PBMCs from two donors subjected to radiation exposure as described in Example 4 and FIG. 18. An ANOVA analysis was performed to characterize the technical and biological variation for each (dose, time) experiment separately. For a given (dose, time) setting, Yᵢⱼₖ denotes the array measurement of the kth technical replicate of the jth blood draw of the ith donor, where k=1, . . . , nᵢⱼ; j=1, . . . , m; i=1, . . . , r

For the in vitro experiment, we have r=2, m=3, n_{if}=2-3. We employed a mixed effect model: Yᵢⱼₖ=μ+αᵢ+βᵢⱼ+εᵢⱼₖ, where μ is the population mean; αᵢ˜N(0, σ²_{α}) represents the individual effect; βᵢⱼ˜N(0, σ²_{β}) represents the blood draw effect; and εᵢⱼₖ˜N(0, σ²) represents the measurement errors in technical replicates.

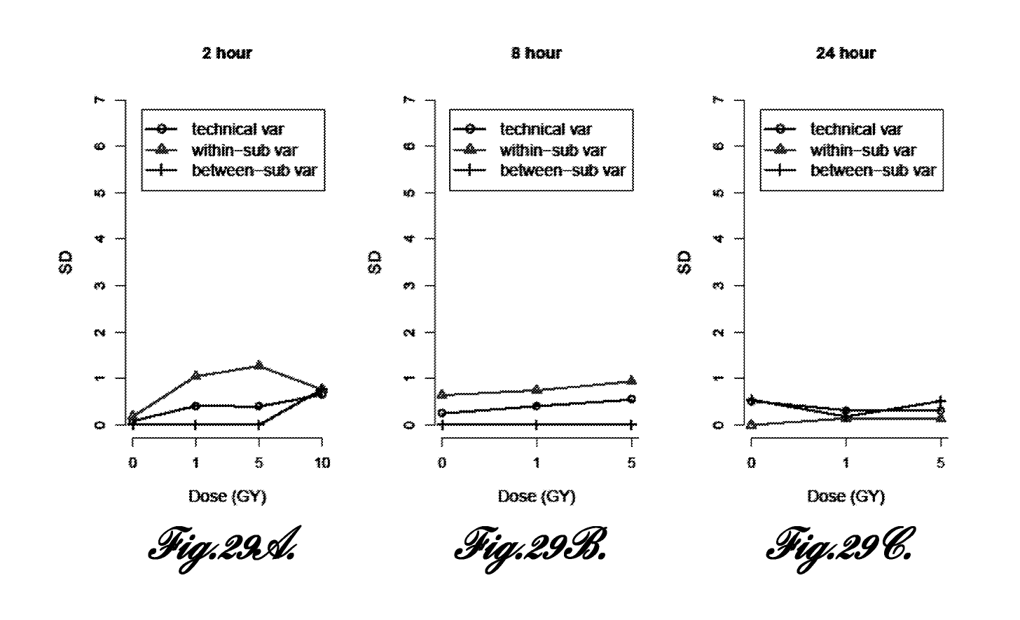

FIG. 28A-C graphically illustrates the assay results for Smc1 pS957. FIG. 29A-C graphically illustrates the assay results for Smc1 p966. The estimated technical variation (σ), within subject variation (σ_{β}) and between subject variation (σ_{α}) are shown for pS957 (FIGS. 28A-C) and pS966 (FIGS. 29A-C) at 2 hours (panel A), 8 hours (panel B) and 24 hours (panel C) after exposure. The results indicate that interindividual variation is substantial for pS957, dwarfing the assay and intraindividual variation.

Discussion

The data described herein demonstrate that the phospho-Smc1 ELISAs are able to distinguish ATM⁺ from ATM⁻ cells (as shown in FIG. 10 and described in Example 2). Consistent with these results, the ANOVA-based analysis of interindividual variation in the phospho-Smc1 response in our healthy blood donors as shown in FIGS. 28A-C and FIGS. 29A-C also showed evidence of significant interindividual differences. These results indicate that radiation exposure of a biological sample obtained from a subject (such as a blood sample) and determination of the phospho-Smc1 response in the exposed biological sample may be used to determine the susceptibility of the subject (from which the sample was obtained) to ionizing radiation exposure.

While illustrative embodiments of the invention have been illustrated and described, it will be appreciated that various changes can be made therein without departing from the spirit and scope of the invention.

The embodiments of the invention in which an exclusive property or privilege is claimed are defined as follows:

1. A method for assessing the exposure of a subject to ionizing radiation comprising measuring the presence or amount of Smc1 protein phosphorylated at least at one of serine 957 or serine 966 in a biological sample obtained from the subject, the method comprising:
(i) contacting the biological sample with a capture reagent that specifically binds to a first epitope on the Smc1 protein;
(ii) contacting the biological sample with at least one detection reagent that specifically binds to phosphorylated serine 957 or phosphorylated serine 966 with reference to human Smc1 protein (SEQ ID NO:6); and
(iii) determining the presence or amount of the bound detection reagent, wherein an increased amount of bound detection reagent in comparison to a reference standard, or an amount of bound detection agent above a reference threshold value indicates that the subject was exposed to ionizing radiation.

2. The method of claim 1, wherein at least one of the capture reagent or the detection reagent is a polyclonal antibody, a monoclonal antibody or a fragment thereof.

3. The method of claim 1, wherein the detection reagent is labeled by a detectable moiety selected from the group consisting of an enzyme, a fluorescent label, a stainable dye, a chemiluminescent compound, a colloidal particle, a radioactive isotope, a near-infrared dye, a DNA dendrimer, a water-soluble quantum dot, a latex bead, a selenium particle, and a europium nanoparticle.

4. The method of claim 1, wherein the subject is a mammal.

5. The method of claim 4, wherein the subject is human.

6. The method of claim 1, wherein the subject is assessed in a time period greater than 30 seconds after suspected exposure to ionizing radiation.

7. The method of claim 1, wherein the ionizing radiation exposure is the result of a nuclear accident or attack.

8. The method of claim 1, wherein exposure to ionizing radiation is the result of a procedure to diagnose or treat a medical condition.

9. The method of claim 1, wherein the biological sample is selected from the group consisting of cultured cells, tissue, blood, plasma, serum, urine, saliva, semen, stool, sputum, cerebral spinal fluid, tears, and mucus, or cells derived therefrom.

10. The method of claim 9, further comprising isolating leukocytes from the biological sample, lysing said leukocytes and contacting the lysate according to steps (i) and (ii) of claim 1.

11. The method of claim 1, wherein the reference standard is a synthetic hybrid reference peptide comprising (i) the first epitope of Smc1 that is bound by the capture reagent and (ii) an epitope comprising serine 957 or phosphorylated serine 966 of the Smc1 protein.

12. The method of claim 1, wherein the method is capable of determining the dose of radiation to which the subject was exposed.

13. The method of claim 12, wherein the method is capable of detecting that the subject was exposed to a dose of ionizing radiation as low as 0.5 Gy.

14. The method of claim 12, wherein the method further comprises categorizing the subject as in need immediate medical care or not in need of immediate medical care, based on the determined exposure to ionizing radiation or determined dose of ionizing radiation to which the subject was exposed.

15. The method of claim 1, wherein the method is one of an ELISA assay, a microsphere-based immunoassay, or a lateral flow test strip.

16. The method of claim 1, wherein the method is a point-of-care lateral flow test strip.

17. The method of claim 16, wherein the subject self-administers the method.

18. A kit for detecting the presence or amount of Smc1 protein phosphorylated at one of serine 957 or serine 966 in a biological sample, the kit comprising:
(i) a capture reagent that specifically binds to a first epitope on the Smc1 protein; and
(ii) at least one detection reagent that specifically binds to a second epitope comprising phosphorylated serine 957 or phosphorylated serine 966 with reference to human Smc1 protein.

19. The kit of claim 18, further comprising a reference standard.

20. The kit of claim 19, wherein the reference standard is a synthetic hybrid reference peptide comprising the first epitope and the second epitope, wherein the synthetic hybrid reference peptide is capable of simultaneously binding to both the capture reagent and the at least one detection reagent.

21. The kit of claim 18, wherein at least one of the capture reagent or the detection reagent is a polyclonal antibody, a monoclonal antibody or a fragment thereof.

22. The kit of claim 21, wherein the capture reagent and the detection reagents are monoclonal antibodies, or fragments thereof.

23. The kit of claim 22, wherein the at least one of said monoclonal antibodies is bound to a microplate or microtiter plate in a format suitable for an Enzyme-Linked Immunosorbent Assay (ELISA).

24. The kit of claim 18, wherein the synthetic reference peptide is a phosphopeptide that is phosphorylated at a serine residue corresponding to serine 957 or serine 966, with reference to the human Smc1 protein (SEQ ID NO:6).

25. The kit of claim 18, wherein the capture reagent binds to an epitope of Smc1 comprising DLTKYPDANPNPNEQ (SEQ ID NO:1).

26. The kit of claim 18, wherein the detection reagent is labeled by a detectable moiety selected from the group consisting of an enzyme, a fluorescent label, a stainable dye, a chemiluminescent compound, a colloidal particle, a radioactive isotope, a near-infrared dye, a DNA dendrimer, a water-soluble quantum dot, a latex bead, a selenium particle, and a europium nanoparticle.

27. A device for point of care detection of exposure to ionizing radiation, wherein the device indicates the presence of Smc1 protein phosphorylated at serine 957 or serine 966 in a biological fluid sample, the device comprising,
(i) a sample receiving zone adapted to receive a biological fluid sample,
(ii) an analyte detection region comprising a porous material which conducts lateral flow of the fluid sample, wherein the analyte detection region comprises an immobile indicator capture reagent that specifically binds to a first epitope on the Smc1 protein; and
(iii) a detection labeling reagent zone comprising a first mobile detection labeling reagent that specifically binds to phosphorylated serine 957 or phosphorylated serine 966 with reference to the Smc1 protein (SEQ ID NO:6), wherein the sample receiving zone is in lateral flow contact with the detection labeling reagent zone and with the analyte detection region.

28. The device of claim 27, wherein at least one of the capture reagent or the detection reagent is a polyclonal antibody, a monoclonal antibody or a fragment thereof.

29. The device of claim 27, wherein the detection reagent is labeled by a detectable moiety selected from the group consisting of an enzyme, a fluorescent label, a stainable dye, a chemiluminescent compound, a colloidal particle, a radioactive isotope, a near-infrared dye, a DNA dendrimer, a water-soluble quantum dot, a latex bead, a selenium particle, and a europium nanoparticle.

30. The device of claim 27, wherein the capture agent specifically binds to an epitope of Smc1 comprising DLTKYPDANPNPNEQ (SEQ ID NO:1).

31. The device of claim 27, wherein the sample receiving zone is adapted to receive between about 100 μL and about 1 mL of biological fluid sample.

32. The device of claim 31, wherein the biological fluid sample is selected from the group cells in liquid culture medium, liquefied tissue, blood, plasma, serum, urine, saliva, semen, liquefied stool, sputum, cerebral spinal fluid, tears, and mucus, or comprises cells derived therefrom.

33. A method of determining the susceptibility of a subject to ionizing radiation exposure, the method comprising:
(a) obtaining one or more biological test sample(s) from a subject;
(b) exposing at least a portion of said biological test sample(s) to one or more predetermined dosages of ionizing radiation; and
(c) determining the presence or amount of Smc1 protein phosphorylated at least at one of serine 957 or serine 966, with reference to human Smc1 protein (SEQ ID NO:6) in the biological sample(s) exposed to radiation in accordance with step (b), wherein the amount or presence phosphorylated Smc1 protein detected in the biological test sample in comparison to a control or reference standard is indicative of the subject's susceptibility to exposure to ionizing radiation.

34. The method of claim 33, wherein the subject is a human subject.

35. The method of claim 33, wherein the biological sample according to step (a) is obtained prior to the exposure of the subject to ionizing radiation.

36. The method of claim 34, wherein the subject is a cancer patient and the method is carried out prior to treatment.

37. The method of claim 33, wherein step (c) comprises:
(i) contacting the biological sample of (b) with a capture reagent that specifically binds to a first epitope on the Smc1 protein;
(ii) contacting the biological sample according to (i) with at least one detection reagent that specifically binds to phosphorylated serine 957 or phosphorylated serine 966; and
(iii) determining the presence or amount of the bound detection reagent.

38. The method of claim 33, wherein step (c) is carried out within 15 minutes to twenty four hours after step (b).

39. The method of claim 33, wherein the biological sample is selected from the group consisting of cultured cells, tissue, blood, plasma, serum, urine, saliva, semen, stool, sputum, cerebral spinal fluid, tears, and mucus, or cells derived therefrom.

40. The method of claim 33, wherein the reference standard is derived from one or more healthy subjects known to not be afflicted with the genetic disorder ataxia telangiectasia (AT), wherein a decrease in the presence or amount of Smc1 phosphorylation detected in the test sample as compared to the reference standard indicates that the subject has an increased susceptibility to ionizing radiation exposure.

41. The method of claim 33, wherein the reference standard is derived from one or more subjects known to be afflicted with the genetic disorder ataxia telangiectasia (AT), and wherein an increase in the presence or amount of Smc1 phosphorylation detected in the test sample as compared to the reference standard indicates that the subject does not have an increased susceptibility to ionizing radiation exposure.
